(19) United States
(12) Patent Application Publication
(10) Pub. No.: US 2003/0073837 A1
(43) Pub. Date: Apr. 17, 2003

(54) 3-HETEROARYLIDENYL-2-INDOLINONE COMPOUNDS FOR MODULATING PROTEIN KINASE ACTIVITY AND FOR USE IN CANCER CHEMOTHERAPY

(76) Inventors: PETER J. LANGECKER, MONTE SERENO, CA (US); LAURA K. SHAWVER, SAN FRANCISCO, CA (US); PENG CHO TANG, MORAGE, CA (US); LI SUN, FOSTER CITY, CA (US)

Correspondence Address: LYON & LYON LLP, 633 WEST FIFTH STREET, SUITE 4700, LOS ANGELES, CA, 90071, US

(21) Appl. No.: 09/476,232

(22) Filed: Dec. 30, 1999

Related U.S. Application Data
(60) Provisional application No. 60/114,313, filed on Dec. 31, 1998.

(51) Int. Cl. C 07D 4 3/02

(52) U.S. Cl. 544/333; 544/238; 544/405

(57) ABSTRACT

The present invention relates to 3-heteroarylidenyl-2-indolinone compounds that modulate the enzymatic activity of protein kinases and therefore are expected to be useful in the prevention and treatment of protein kinase related cellular disorders such as cancer. Furthermore, these compounds are expected to enhance the efficacy of other chemotherapeutic agents, in particular, fluorinated pyrimidines, in the treatment of cancer.

### INTRODUCTION

The present invention relates generally to chemistry, biochemistry, pharmacology, medicine and cancer treatment. More particularly, it relates to 3-heteroarylidenyl-2-indolinone compounds that modulate the activity of protein kinases (PKs) and to methods for their use in treating disorders related to abnormal protein kinase activity including cancer wherein combinations of the compounds with other chemotherapeutic agents are used.

### BACKGROUND OF THE INVENTION

The following is provided by way of background information only and is not admitted to be or to describe prior art to the present invention.

PKs are enzymes that catalyze the phosphorylation of hydroxy groups on tyrosine, serine and threonine residues of proteins. The consequences of this seemingly simple activity are staggering; cell growth, differentiation and proliferation; i.e., virtually all aspects of cell life, in one way or another depend on PK activity. Furthermore, abnormal PK activity has been related to a host of disorders, ranging from relatively non-life threatening diseases such as psoriasis to extremely virulent diseases such as glioblastoma (brain cancer).

The PKs can conveniently be broken down into two classes, the protein tyrosine kinases (PTKs) and the serine-threonine kinases (STKs).

One of the prime aspects of PK activity is involvement with growth factor receptors. Growth factor receptors are cell-surface proteins. When bound by a growth factor ligand, growth factor receptors are converted to an active form which interacts with proteins on the inner surface of a cell membrane. This leads to phosphorylation on tyrosine residues of the receptor as well as other proteins and to the formation inside the cell of complexes with a variety of cytoplasmic signaling molecules. These complexes, in turn, affect numerous cellular responses such as cell division (proliferation), cell differentiation, cell growth, expression of metabolic effects on the extracellular microenvironment, etc. For a more complete discussion, see Schlessinger and Ullrich, Neuron, 1992, 9:303-391 which is incorporated by reference, including any drawings, as if fully set forth herein.

Growth factor receptors with PK activity are known as receptor tyrosine kinases (“RTKs”). They comprise a large family of transmembrane receptors with diverse biological activity. At present, at least nineteen (19) distinct subfamilies of RTKs have been identified. An example of these is the subfamily designated the “HER” RTKs, which includes EGFR (epithelial growth factor receptor), HER2, HER3 and HER4. These RTKs consist of an extracellular glycosylated ligand binding domain, a transmembrane domain and an intracellular cytoplasmic catalytic domain that can phosphorylate tyrosine residues on proteins.

Another RTK subfamily consists of insulin receptor (IR), insulin-like growth factor I receptor (IGF-1R) and insulin receptor related receptor (IRR). IR and IGF-1R interact with insulin, IGF-I and IGF-II to form a heterotetramer composed of two entirely extracellular glycosylated α subunits and two β subunits which cross the cell membrane and which contain the tyrosine kinase domain.

A third RTK subfamily is referred to as the platelet derived growth factor receptor (“PDGFR”) group, which includes PDGFRα, PDGFRβ, CSFIR, c-kit and c-fms. These receptors consist of glycosylated extracellular domains composed of variable numbers of immunoglobin-like loops and an intracellular domain wherein the tyrosine kinase domain is interrupted by unrelated amino acid sequences.

Another group which, because of its similarity to the PDGFR subfamily, is sometimes subsumed in the later group, is the fetus liver kinase (“flk”) receptor subfamily. This group is believed to be composed of kinase insert domain-receptor fetal liver kinase-1 (KDR/FLK-1), flk-1R, flk-4 and fms-like tyrosine kinase 1 (flt-1).

One further member of the tyrosine kinase growth factor receptor family is the fibroblast growth factor (“FGF”)receptor group. This group consists of four receptors, FGFR1-FGFR4, and seven ligands, FGF1-FGF7. While not yet well characterized, it appears that the receptors also consist of a glycosylated extracellular domain containing a variable number of immunoglobin-like loops and an intracellular domain in which the PTK sequence is interrupted by regions of unrelated amino acid sequences.

A more complete listing of the known RTK subfamilies is described in Plowman et al., DN&P, 1994, 7(6):334-339 which is incorporated by reference, including any drawings, as if fully set forth herein.

In addition to the RTKs, there also exists a family of entirely intracellular PTKs called “non-receptor tyrosine kinases” or “cellular tyrosine kinases.” This latter designation, abbreviated “CTK”, will be used herein. CTKs do not contain extracellular and transmembrane domains. At present, over 24 CTKs in 11 subfamilies (Src, Frk, Btk, Csk, Abl, Zap70, Fes, Fps, Fak, Jak and Ack) have been identified. The Src subfamily appear so far to be the largest group of CTKs and includes Src, Yes, Fyn, Lyn, Lck, Blk, Hck, Fgr and Yrk. For a more detailed discussion of CTKs, see Bolen, Oncogene, 1993, 8:2025-2031, which is incorporated by reference, including any drawings, as if fully set forth herein.

The serine-threonine kinases or STKs, like the CTKs, are predominantly intracellular although there are a few STK receptor kinases. STKs are the most common of the cytosolic kinases; i.e., kinases which perform their function in that part of the cytoplasm other than the cytoplasmic organelles and cytoskelton. The cytosol is the region within the cell where much of the cell's intermediary metabolic and biosynthetic activity occurs; e.g., it is in the cytosol that proteins are synthesized on ribosomes.

RTKs, CTKs and STKs have all been implicated in a host of pathogenic conditions including, significantly, cancer. Others pathogenic conditions which have been associated with PTKs include, without limitation, psoriasis, hepatic cirrhosis, diabetes, atherosclerosis, angiogenesis, restenosis, ocular diseases, rheumatoid arthritis and other inflammatory disorders, autoimmune disease and a variety of renal disorders.

With regard to cancer, two of the major hypotheses advanced to explain the excessive cellular proliferation that drives tumor development relate to functions known to be PK regulated. That is, it has been suggested that malignant cell growth results from a breakdown in the mechanisms that control cell division and/or differentiation. It has been shown that the protein products of a number of proto-oncogenes are involved in the signal transduction pathways that regulate cell growth and differentiation. These protein products of proto-oncogenes include the extracellular growth factors, transmembrane growth factor PTK receptors (RTKs), cytoplasmic PTKs (CTKs) and cytosolic STKs, discussed above.

Cancer continues to be one of the leading causes of death in human beings. The majority of cancers are solid tumor cancers such as, without limitation, ovarian cancer, colorectal cancer, brain cancer, liver cancer, kidney cancer, stomach cancer, prostate cancer, lung cancer, thyroid cancer, Kaposi's sarcoma and skin cancer. Of the solid tumor cancers, colorectal cancer is a particularly common malignancy; adenocarcinoma of the large bowel affects about one person in 20 in the United States and in most Westernized countries. In the United States, colorectal cancer represents about 15% of all newly diagnosed cancers.

While colorectal cancer is the third leading cause of cancer-related death, prognosis and outcome is highly dependent on the stage the disease at diagnosis. If diagnosed in early stages, colorectal cancer is highly curable using a multidisciplinary treatment regime. Nevertheless, 20-25% of patients diagnosed with the disease will present with metastases or will develop locally recurrent or metastatic disease; the majority of these patients will eventually die of the disease.

The primary modes of treatment of solid tumor cancers, including colorectal cancer, are surgery, radiation therapy and chemotherapy, separately and in combination.

Although the initial formation and growth of tumors does not require new blood vessel formation, any further growth does require neovascularization. That is, for tumors to grow beyond 3 to 4 mm³ in volume, new blood vessel growth; i.e., angiogenesis, the sprouting of new capillaries from existing blood vessels, must occur. In fact, immunohistochemical analysis of tumor sections from the margins of growing tumors show a preponderance of blood vessels, irrespective of tumor type. To accomplish this neovascularization, angiogenic factors are released from hypoxic tumor cells and migrate to nearby blood vessel endothelial cells, activating these cells to undergo morphologic changes, to move and to divide. Tumors that lack adequate vasculature become necrotic (Brem, S., et al., Cancer Res., 1976, 36, 2807-12) and/or apoptotic (Holmgren, L., et al., Nature Med., 1995, 1:149-53; Parangi, S., et al., Cancer Res., 1995, 55:6071-6), whereas tumors which have undergone neovascularization not only can enter a phase of rapid growth but also demonstrate increased metastatic potential. In support of the significance of angiogenesis in human tumors, recent studies relating the angiogenic phenotype and survival in people have shown that the number of microvessels in a primary tumor has prognostic significance in breast carcinoma (Gasparini, G., and Harris, A. L., J. Clin. Oncol., 1995, 13:765-82; Toi, M., et al., Japan. J. Cancer Res., 1994, 85:1045-9), bladder carcinomas (Dickinson, A. J., et al., Br. J. Urol., 1994, 74:762-6), colon carcinomas (Ellis, L. M., et al., Surgery, 1996, 120(5):871-8) oral cavity tumors (Williams, J. K., et al., Am. J. Surg., 1994, 168:373-80). Angiogenesis may also play a role in the growth of hematopoietic neoplasms and multiple myeloma (Bellamy, W. T., et al., Proc. Amer. Assoc. Cancer Res., 1998, Abstract #2566.

At present, the central mediator of malignant tumor angiogensis is thought to be the endothelial mitogen, vascular endothelial growth factor (VEGF). VEGF is mitogenic for many types of small and large vessel endothelial cells. It induces the production of tissue factors, collagenase and plasminogen activators and inhibitors. VEGF is sometimes referred to as “vascular permeability factor” by virtue of its permeability enhancing effects (Landriscina, M., et al., Brit. J. Cancer, 1998, 78(6):765-770). In fact, the vascular permeability factor potency of VEGF is some 50,000 times higher than that of histamine which is a well-known vascular permeabilizing molecule (Dvorak, H. F., et al., Am. J. Path., 1995, 146:1029-39). This increased permeability results in extravasion of macromolecules such as fibrogen from the circulation which provides a fibrin gel meshwork or substratum for the migration and organization of endothelial cells as well as tumor cells (Kumar, H., et al., Clin. Cancer Res., 1998, 4:1279-85. VEGF expression has been demonstrated in vitro in a number of human cancer cells lines and surgically in resected tumors of the gastrointestinal tract, ovary, brain, breast and kidney (Thomas, K. A., J. Biol. Chem., 1996, 271:603-6).

VEGF has also been closely associated with the development of colorectal cancer; i.e, increased levels of VEGF have been found in tumor tissue from patients with colorectal cancer. In fact, a strong correlation has been observed between the increases VEGF and the stage and depth of intestinal wall invasion (C. Barone, et al., Brit. J. Cancer, 1998, 78(6):765-70). Consistent with this result is the finding that serum levels of VEGF correlate significantly with Dukes stage and carcinoembryotic antigen levels and that patients with hepatic and/or lymph node metastases tend to show higher serum VEGF levels than those patients without such metastases (Fujisaki, K., et al., Am. J. Gastroenterology, 1998, 93(2):249-52).

Given the necessity of neovascularization for the growth of solid tumors and the role of VEGF as one of the most important mediators of angiogenesis, particularly in colorectal cancer, compounds capable of inhibiting the angiogenic effect of VEGF would be expected to retard the rebound effect observed with fluorouracil-based colorectal cancer treatment and thereby increase the chemotherapeutic efficacy of fluorouracil, with or without leucovorin. An additional advantage to such a method might be that the use of an angiogenic inhibitor that reduces the ability of the tumor to develop new blood vessels and thus would be cytostatic rather than cytotoxic may compliment standard cytotoxic chemotherapy; that is, utilize different mechanisms of action to increase the efficacy of the cytotoxic agent without additional toxicity.

### SUMMARY OF THE INVENTION

Our search for small organic molecules which modulate protein kinase mediated signal transduction has resulted in the discovery of 3-heteroarylidenyl-2-indolinones which modulate the activity of protein kinases (PKs) such as receptor tyrosine kinases (RTKs), cellular tyrosine kinases (CTKS) and serine-threonine tyrosine kinases (STKs). The RTKs include, among others, Flk-1, Flt-1, Tie-1 and Tie-2, all of whose expression have been found to be restricted to endothelial cells. Of particular significance with regard to the present invention is the fact that Flk-1 is believed to play a critical role in angiogenesis and that that role is mediated by VEGF. This suggests that 3-heteroarylidenyl-2-indolinones should be capable of inhibiting VEGF-mediated vascularization, and thereby the growth, of tumors during the period when no chemotherapeutic agent, such as, without limitation, a fluorinated pyrimidine, is being administered to a patient and thus should increase the efficacy of the chemotherapeutic agent.

Thus, in one aspect, the present invention relates to a 3-heteroarylidenyl-2-indolinone compound that inhibits angiogenesis or vasculogenesis in a cell, the compound having the chemical structure:

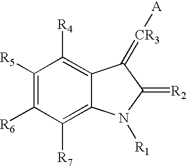

or a pharmaceutically acceptable salt or prodrug thereof, wherein

R₁ is H or alkyl;

R₂ is O or S;

R₃ is hydrogen;

R₄, R₅, R₆, and R₇ are each independently selected from the group consisting of hydrogen, alkyl, alkoxy, aryl, aryloxy, alkaryl, alkaryloxy, halogen, trihalomethyl, S(O)R, SO₂NRR′, SO₃R, SR, NO₂, NRR′, OH, CN, C(O)R, OC(O)R, (CH₂)ₙCO₂R, and CONRR′;

A is a five membered heteroaryl ring selected from the group consisting of thiophene, pyrrole, pyrazole, imidazole, 1,2,3-triazole, 1,2,4-triazole, oxazole, isoxazole, thiazole, isothiazole, 2-sulfonylfuran, 4-alkylfuran, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole, and tetrazole, optionally substituted at one or more positions with alkyl, alkoxy, aryl, aryloxy, alkaryl, alkaryloxy, halogen, trihalomethyl, S(O)R, SO₂NRR′, SO₃R, SR, NO₂, NRR′, OH, CN, C(O)R, OC(O)R, (CH₂)ₙCO₂R, or CONRR′;

n is 0-3; and,

R and R′ are independently selected from the group consisting of alkyl or aryl.

“Alkyl” refers to a straight-chain, branched or cyclic saturated aliphatic hydrocarbon. Preferably, the alkyl group has 1 to 12 carbons. More preferably, it has from 1 to 7 carbons and most preferably, it is a lower alkyl having from 1 to 4 carbons. Typical alkyl groups include methyl, ethyl, propyl, isopropyl, butyl, isobutyl, tertiary butyl, pentyl, hexyl, and the like. The alkyl group may be optionally substituted with one or more substituents selected from the group consisting of hydroxyl, —C(O)OR, cyano, unsubstituted alkoxy, ═O, ═S, NO₂, halogen, NRR′ and SR.

“Alkenyl” refers to an alkyl group containing at least one carbon-carbon double bond.

“Alkynyl” refers to an alkyl group containing at least one carbon-carbon triple bond.

“Alkoxy” refers to an “-Oalkyl” group wherein the alkyl group may be optionally substituted with one or more halo groups.

“Aryl” refers to a group having at least one aromatic ring structure; that is, a one ring having a conjugated pi electron system and includes carbocyclic aryl, heterocyclic aryl and biaryl groups. The aryl group may be optionally substituted with one or more substituents selected from the group consisting of halogen, trihalomethyl, hydroxyl, SR, nitro, cyano, alkoxy, alkyl and NRR′.

“Alkaryl” refers to an alkyl that is covalently joined to an aryl group. Preferably, the alkyl is an unsubstituted lower alkyl.

“Carbocyclic aryl” refers to an aryl group wherein the ring atoms are carbon.

“Heterocyclic aryl” refers to an aryl group having from 1 to 3 heteroatoms as ring atoms, the remainder of the ring atoms being carbon. Heteroatoms include oxygen, sulfur, and nitrogen. The ring may be five-membered or six-membered. Examples of heterocyclic aryl groups include furanyl, thienyl, pyridyl, pyrrolyl, N-alkylpyrrolyl, pyrimidyl, pyrazinyl, imidazolyl and the like.

“Amide” refers to —C(O)NHR^{a}, where R^{a} is alkyl, aryl, alkylaryl or hydrogen.

“Thioamide” refers to —C(S)NHR^{a}

“Amino” refers to an NRR′ group in which both R and R′ are hydrogen.

“Thioether” refers to an —SR^{b} group wherein R^{b} is alkyl, aryl or alkylaryl.

“Halogen” refers to fluorinem chlorine, bromine or iodine.

“Sulfonyl” refers to —S(O)₂R^{c}, where R^{c} is aryl, —C(CN)═C— aryl, —CH₂CN, alkyaryl, —SO₂NRR′, —NH(alkyl), —NH(alkylaryl), or —NH(aryl).

Physiologically acceptable salts and prodrugs of the 3-heteroarylidenyl-2-indolinones are also within the scope of this invention.

A “physiologically acceptable salt” refers to a salt that is non-deleterious to the physical well-being of a patient to whom it is administered. The physiologically acceptable salts which the compounds of this invention may form include negatively or the positively charged species. Examples of salts in which the compound forms the positively charged moiety include, without limitation, quaternary ammonium (defined elsewhere herein), salts such as the hydrochloride, sulfate, carbonate, lactate, tartrate, maleate, succinate wherein the nitrogen atom of the quaternary ammonium group is a nitrogen of the selected compound of this invention which has reacted with the appropriate acid. Salts in which a compound of this invention forms the negatively charged species include, without limitation, the sodium, potassium, calcium and magnesium salts formed by the reaction of a carboxylic acid group in the compound with an appropriate base (e.g. sodium hydroxide (NaOH), potassium hydroxide (KOH), Calcium hydroxide (Ca(OH)₂), etc.).

A “prodrug” refers to an agent which is converted into the parent drug in vivo. Prodrugs are often useful because, in some situations, they may be easier to administer than the parent drug. They may, for instance, be bioavailable by oral administration whereas the parent drug is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water solubility is beneficial. A further example of a prodrug might be a short polypeptide bonded to a carboxy group wherein metabolic removal of the polypeptide group releases the active compound.

The 3-heteroarylidenyl-2-indolinone compounds of this invention may exist as the E or the Z isomers of a combination thereof. All of these configurations are within the scope of this invention. In preferred embodiments of this invention, the 3-heteroarylidenyl-2-indolinones are predominantly (greater than 90%) the Z-isomer.

By “inhibit” is meant eliminate, reduce, contain, impede, prevent, slow, retard and/or restrict. In a presently preferred embodiment of this invention, inhibit refers to the inhibition of angiogenesis or vasculogenesis.

By “angiogenesis” activity is meant the formation of new blood vessels in a tissue.

By “vasculogenesis” is meant the spread of new blood vessels through a tissue to form a vascular system.

In another aspect, the 3-heteroarylidenyl-2-indolinone compound of this invention is 3-[4-(2-carboxyethyl-3,5-dimethylpyrrol-2-yl)methylidenyl]-2-indolinone (Structure 1).

The 3-heteroarylidenyl-2-indolinone is 3-[(2,4-dimethylpyrrol-5-yl)methylidenyl]-2-indolinone (Structure 2) in yet another aspect of this invention.

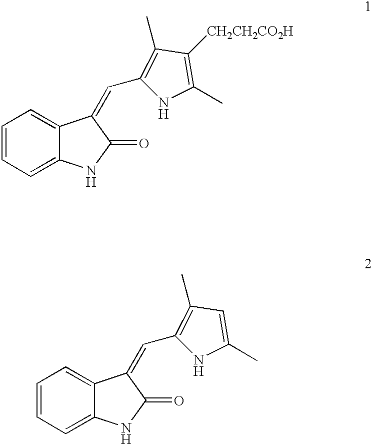

In a further aspect of this invention, a method is provided for treating cancer comprising administering to a patient in need of such treatment a therapeutically effective amount of another chemotherapeutic agent and a therapeutically effective amount of a 3-heteroarylidenyl-2-indolinone wherein the 3-heteroarylidenyl-2-indolinone has the chemical structure:

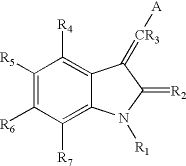

wherein R₁, R₂, R₃, R₄, R₅, R₆, R₇ and A are the same as set forth above.

In a presently preferred embodiment, the chemotherapeutic agent is a fluorinated pyrimidine.

Again, physiologically acceptable salts or prodrugs of the 3-heteroarylidenyl-2-indolines are within the scope of this combination chemotherapy aspect of the present invention.

The term “method” refers to manners, means, techniques and procedures for accomplishing a given task including, but not limited to, those manners, means, techniques and procedures either known to, or readily developed from known manners, means, techniques and procedures by, practitioners of the chemical, pharmacological, biological, biochemical and medical arts.

With regard to cancer, the term “treating” simply means that the life expectancy of an individual affected with a cancer will be increased, that one or more of the symptoms of the disease will be reduced and/or that quality of life will be enhanced.

As used herein, “administer,” “administering” or “administration” refers to the delivery of a compound, salt or prodrug of the present invention or of a pharmaceutical composition containing a compound, salt or prodrug of this invention to a patient for the purpose of treatment of cancer or the prevention or treatment of a PK-related disorder.

“Comprising” as used herein in connection with “administering” is intended to mean that drugs being administered pursuant to the present invention may be administered as simply a combination of a 3-heteroarylidenyl-2-indolinone compound and a chemotherapeutic agent alone or may be expanded to include additional drugs, such as, when the chemotherapeutic agent is a fluorinated pyrimidine, leucovorin, which are known or expected to offer additional beneficial characteristics to the combination.

In general, a “therapeutically effective amount” refers to that amount of a drug or its metabolite which is effective to prevent, alleviate, reduce or ameliorate symptoms of disease or prolong the survival of the patient being treated. More particularly, in reference to the treatment of cancer, a therapeutically effective amount refers to that amount which has the effect of (1) reducing the size of (or preferably eliminating) the tumor; (2) inhibiting (that is, slowing to some extent, preferably stopping) tumor metastasis; (3) inhibiting to some extent (that is slowing to some extent, preferably stopping) tumor growth; and/or, (4) relieving to some extent (or preferably eliminating) one or more symptoms associated with the cancer.

In addition to the above general definition, by a “therapeutically effective amount” of a chemotherapeutic agent is meant any amount administered in any manner and in any treatment regime as may be currently recognized in the medical arts or as may come about as the result of future developments regarding the use of these agents. In a presently preferred embodiment of this invention, the chemotherapeutic agent is a fluorinated pyrimidine, in particular, fluorouracil, and the treatment regimes are those known in the chemotherapeutic art for the administation of fluorouracil.

A “treatment regime” refers to specific quantities of selected chemotherapeutic agents (and, optionally, other agent such as the 3-heteroarylidenly-2-indolinone compound of this invention) administered at set times in a set manner over an established time period. For example, without limitation, a common treatment regime for treating colorectal cancer with fluorouracil/leucovorin comprises administering 425 mg/m² (milligrams per square meter of body surface area, a manner of measuring chemotherapeutic agent dosage well known to those skilled in the art) flourouracil plus 20 mg/m² leucovorin (specific quantities of selected agents) daily for 5 days (set times) by intravenous push (set manner) repeated at 4 to 5 week intervals (established time period).

When referring to “set times” of administration within a treatment regime, “consecutive days” means consectutive calendar days; i.e., Monday, Tuesday, Wednesday, etc.

“Staggered” days means calendar days with other calendar days between them, e.g., without limitation, Monday, Wednesday, Saturday, etc.

Furthermore, with regard to a “therapeutically effective amount of a 3-heteroarylidenyl-2-indolinone,” the phrase refers to an amount of the compound sufficient to inhibit the growth, size and vascularization; i.e., angiogenesis and/or vasculogenesis, of tumors during the “recovery” periods, i.e., the periods in a treatment regime when no other chemotherapeuic agent is being administered to a patient.

A “patient” refers to any higher organism which is susceptible to a PK related disorder including in particular cancer. Preferentially, “patient” refers to a mammal, especially a human being.

“Fluorinated pyrimidine chemotherapeutic agents” are well known to those skilled in chemotherapeutic art; examples, without limitation, of fluorinated pyrimidines which may be used with the compounds of this invention include, without limitation, carmofur, doxifluridine, fluorouracil, floxuridine, tegafur, capecitabine and uracil-ftorafur (UFT).

In a presently preferred embodiment of this invention, the fluorinated pyrimidine chemotherapeutic agent is fluorouracil.

It is also a presently preferred embodiment of this invention that, when the fluorinated pyrimidine chemotherapeutic agent is fluorouracil, the above method for the treatment of cancer also comprises leucovorin.

The 3-heteroarylidenyl-2-indolinone used to treat cancer incombination with other chemotherapeutic agents is selected from the group consisting of 5-hydroxy-3-[(2,4-dimethylpyrrol-5-yl)methylidenyl]-2-indolinone (Structure 3), 4-methyl-5-(2-oxo-1,2-dihydroindol-3-ylidenemethyl)-1H-pyrrole-2-carboxylic acid (Structure 4), 4-methyl-5-(2-oxo-1,2-dihydroindol-3-ylidenemethyl)-1H-pyrrole-2-carboxylic acid methyl ester (Structure 5), 3-(5-hydroxymethyl-3-methyl-1H-pyrrol-2-ylmethylene)-1,3-dihydroindole-2-one (Structure 6) and 4-methyl-5-(2-oxo-1,2-dihydroindol-3-ylidenemethyl)-1H-pyrrole-2-carbaldehyde (Structure 7) in yet another aspect of this invention.

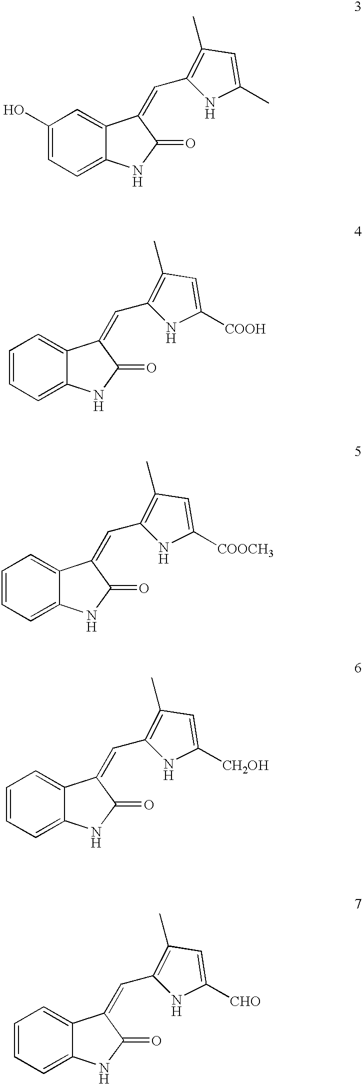

In a further aspect of this invention, the 3-heteroarylidenly-2-indolinone compound used to treat cancer in combination with other chemotherapeutic agents is 3-[4-(2-carboxyethyl-3,5-dimethylpyrrol-2-yl)methylidenyl]-2-indolinone (Structure 1).

In a still further aspect of this invention, the 3-heteroarylidenyl-2-indolinone compound used to treat cancer in combination with other chemotherpeutic agents is 3-[(2,4-dimethylpyrrol-5-yl)methylidenyl]-2-indolinone (Structure 2).

The cancer which may be treated using the above-described method may be selected from the group consisting of breast cancer, gastric cancer, ovarian cancer, renal cancer, hepatic cancer, pancreatic cancer, bladder cancer, thyroid cancer, prostate cancer and colorectal cancer.

Yet another aspect of this invention is a method for treating cancer comprising administering to a patient in need of such treatment a therapeutically effective amount of fluorouracil and a therapeutically effective amount of a compound selected from the group consisting of 3-[4-(2-carboxyethyl-3,5-dimethylpyrrol-2-yl)methylidenyl]-2-indolinone and 3-[(2,4-dimethylpyrrol-5-yl)methylidenyl]-2-indolinone.

In a preferred embodiment, the cancer is colorectal cancer.

In another aspect of this invention, the above method for the treatment of cancer includes the use of leucovorin.

The therapeutically effective amount of fluorouracil comprises from about 300 to about 800 mg/m², preferably from about 400 to about 500 mg/m² of the compound.

The therapeutically effective amount of fluoruracil may be administered as an intravenous bolus injection or as a continuous intravenous infusion in yet another aspect of this invention.

The therapeutically effective amount of 3-[(2,4-dimethylpyrrol-5-yl)-methylidenyl]-2-indolinone comprises from about 4 to about 190 mg/m², preferrably from about 72 to 145 mg/m² of the compound.

The therapeutically effective amount of leucovorin comprises from about 20 to about 500 mg/m² preferrably from about 20 to about 200 mg/m² of the compound.

A still further aspect of this invention is a treatment regime comprising the administration of from about 400 to about 500 mg/m² fluorouracil on one or more days, which may be consecutive or staggered, after which from about 72 to about 145 mg/m² 3-[(2,4-dimethylpyrrol-5-yl)methylidenyl]-2-indolinone are administered on one or more days, which days likewise may be consecutive or staggered.

In another aspect, 20 mg/m² leucovorin may also be adminstered on the days on which fluorouracil is administered.

In a presently preferred embodiment of this invention, the above treatment regime is a four week treatment regime, fluorouracil (and, optionally, leucovorin) being administered as an intravenous bolus injection on days 1, 2, 3, 4 and 5 of the first week of the treatment regime while the 3-[(2,4-dimethylpyrrol-5-yl)methylidenyl]-2-indolinone being administered as an intravenous bolus injection twice a week during weeks 2, 3 and 4 of the treatment regime.

Another aspect of this invention is a method for treating cancer comprising administering to a patient in need of such treatment a therapeutically effective amount of 3-[(2,4-dimethylpyrrol-5-yl)methylidenyl]-2-indolinone and a therapeutically effective amount of gemcitabine, another fluoropyrimidine compound. Gemcitabine has shown particular effectiveness in the treatment of advanced pancreatic cancer. Furthermore, in combination with other chemotherapeutic agents, e.g., paclitaxel, carboplatin, doxorubicin (in particular, liposomal doxorubicin) and topotecan, gemcitabine has shown substantial activity against other refractory solid tumor cancers including advanced ovarian cancer, small cell lung cancer and kidney cancer. The combination of gemcitabine with 3-[(2,4-dimethylpyrrol-5-yl)methylidenyl]-2-indolinone, alone or in further combination with additional chemotherapeutic agents such as those indentified above, should, for the reasons discussed with regard to fluoropyrimidines generally, provide additional solid tumor inhibiting capacity without adding further toxicity.

Combinations of 3-[(2,4-dimethylpyrrol-5-yl)methylidenyl]-2-indolinone with nucleoside analogs other than gemcitabine are also contemplated by the present invention.

Another pyrimidine analog which should benefit from combination with 3-[(2,4-dimethylpyrrol-5-yl)methylidenyl]-2-indolinone is capecitabine which has shown effectiveness against metastatic breast cancer; such a combination is another aspect of this invention.

In addition, the chemotherapeutic combination of 3-[(2,4-dimethylpyrrol-5-yl)methylidenyl]-2-indolinone with either of the pyrimidine chemotherapeutic agents 5-FU or UFT or derivatives, analogs or agents related thereto, is an aspect of this invention.

A further aspect of this invention is the combination of 3-[(2,4-dimethylpyrrol-5-yl)methylidenyl]-2-indolinone with carboplatin, oxaliplatin, cisplatin or related chemotherapeutic agents. Carboplatin and cisplatin are presently the pre-eminent drugs for the treatment of advanced ovarian cancer while oxaliplatin is a first-line chemotherapeutic agent in metastatic colorectal cancer. The use of 3-[(2,4-dimethylpyrrol-5-yl)methylidenyl]-2-indolinone in combination with carboplatin or cisplantin may permit a reduction in the amount of these two very toxic chemotherapeutic agents necessary to treat the cancer. In addition, combination of carboplatin or cisplatin with paclitaxel has shown promise in the treatment of ovarian cancer. The addition of 3-[(2,4-dimethylpyrrol-5-yl)methylidenyl]-2-indolinone to this combination of chemotherapeutic agents could result in the same advantages discussed with regard to the above combinations. A presently preferred chemotherapeutic combination is comprised of 3-[(2,4-dimethylpyrrol-5-yl)methylidenyl]-2-indolinone, cisplatin and gemcitabine.

A further aspect of this invention is the combination of 3-[(2,4-dimethylpyrrol-5-yl)methylidenyl]-2-indolinone with paclitaxel (taxol), its synthetic analog docetaxel or polyglutamated taxanes. Paclitaxel has been approved by the FDA for the treatment of ovarian, breast, lung and AIDS-related cancers. Paclitaxel/docetaxel work by a different mechanism than the compounds of this invention, that is, they block a cell's ability to break down the mitotic spindle during mitosis. Thus, these drugs with their particular mode of action, combined with the anti-angiogenetic activity of 3-[(2,4-dimethylpyrrol-5-yl)methylidenyl]-2-indolinone, could result in a potent tumoricidal/tumoristatic combination.

Yet another aspect of this invention is the combination of 3-[(2,4-dimethylpyrrol-5-yl)methylidenyl]-2-indolinone with CPT11 (irinotecan), a derivative of campothecin that is a topoisomerase I inhibitor and which has proven effective against colorectal cancer. Combination therapies with chemotherapeutic agents related to CPT11 are also contemplated by this invention. Again, the combination of modes of action could be of substantial benefit in the treatment of this form of cancer.

A still further aspect of this invention is the combination of 3-[(2,4-dimethylpyrrol-5-yl)methylidenyl]-2-indolinone with thalidomide which is showing substantial chemotherapeutic utility particularly against refractory myelomas but also against glioblasoma multiforma, an extremely virulent brain cancer. Other cancer which may be responsive to this combination include prostate, breast and skin (Kaposi's sarcoma) cancers.

An aspect of this invention is a chemotherapeutic combination of 3-[(2,4-dimethylpyrrol-5-yl)methylidenyl]-2-indolinone with COX-2 inhibitors. The inhibition of cyclooxygenase-2 prevents production of factors that prompt angiogenesis. The combination would provide a two way attack on the vascularization essential to the vitality of cancer cells.

A combination therapy consisting of 3-[(2,4-dimethylpyrrol-5-yl)methylidenyl]-2-indolinone and tamoxifen or derivatives thereof is as aspect of this invention. Tamoxifen interferes with the activity of estrogen which has been shown to promote the growth of breast cancer cells. The combination of 3-[(2,4-dimethylpyrrol-5-yl)methylidenyl]-2-indolinone, an anti-angiogenesis compound, with this “anti-estrogen” compound could provide a potent additional treatment for breast cancer.

Another aspect of this invention is the combination of 3-[(2,4-dimethylpyrrol-5-yl)methylidenyl]-2-indolinone with leuprolide, a synthetic nonapeptide analog of naturally ocurring gonadotropin-releasing hormone that has demonstrated effectiveness particularly aginst testicular cancer but also against ovarian and breast cancer. Combination therapy using agents related to leuprolide is also contemplated by this invention. Again, a substantial benefit could be gained by combining the two different mode of action compounds.

The chemotherapeutic combination of 3-[(2,4-dimethylpyrrol-5-yl)methylidenyl]-2-indolinone with angiostatin, endostatin or similar chemotherapeutic agents, which inhibit angiogenesis by apoptosis, is likewise an aspect of this invention. Apoptosis is programmed cell death. The combination of cell-killing anti-angiogenesis with cell stasis anti-angiogenesis could be a powerful chemotherapeutic combination.

In addition, a chemotherapeutic combination of 3-[(2,4-dimethylpyrrol-5-yl)methylidenyl]-2-indolinone with a matrix metalloprotease inhibitor. MMPs having been shown to be involved in many disease states including cancer. MMP inhibitors, such as, without limitation, AG3340, are showing tumoristatic efficacy againt solid tumor cancers such as non-small cell lung cancer and hormone-refractory prostate cancer. The addition of an angiogenesis inhibitor could provide a synergistic combination.

The combination of 3-[(2,4-dimethylpyrrol-5-yl)methylidenyl]-2-indolinone with an interferon is another aspect of this invention. Interferon alpha, and its various subtypes (e.g., without limitation, interferons alpha A/2a, alpha/2b, alpha B2/alpha 8) are well-established chemotherapeutic agents against such cancers as hairy-cell leukemia, chronic myeloid leukemia, kidney cancer, melanoma, low grade lymphomas, multiple myeloma and Kaposi's sarcoma.

A further aspect of this invention is the chemotherapeutic combination of 3-[(2,4-dimethylpyrrol-5-yl)methylidenyl]-2-indolinone with doxorubicin, daunorubicin and other anthracycline antineoplastic antibiotic, and derivatives and formulations thereof such as, without limitation, liposomal doxorubicin. Doxorubicin is widely used in the treatment of malignant lymphomas, leukemias, squamous cell cancer of the head and neck, breast cancer and thyroid cancer. Liposomal doxorubicin has been approved for the treatment of Kaposi's sarcoma. Tumor cells weakened by the anti-angiogenesis activity of 3-[(2,4-dimethylpyrrol-5-yl)methylidenyl]-2-indolinone could be much more susceptible to doxorubicin. Combination therapy using 3-[(2,4-dimethylpyrrol-5-yl)methylidenyl]-2-indolinone and metoxantrone, a related chemotherapeutic agent, is specifically contemplated by this invention.

Another chemotherapeutic combination which is an aspect of this invention is the combination of 3-[(2,4-dimethylpyrrol-5-yl) methylidenyl]-2-indolinone with estramustine and chemotherapeutic agents related thereto, which has shown particular utility in the treatment of refractory prostate cancer. Estramustine causes cell death by interferring with DNA synthesis. Again the combination of differing modes of action, DNA synthesis disruption and anti-angiogenesis could provide a useful chemotherapeutic combination.

Combination therapy using 3-[(2,4-dimethylpyrrol-5-yl)methylidenyl]-2-indolinone and the vinca alkaloids including, without limitation, vincristine and vinblastine is also contemplated by the present invention.

A further aspect of this invention is a 3-heteroarylidenyl-2-indolinone selected from the group consisting of 5-hydroxy-3-[(2,4-dimethylpyrrol-5-yl)methylidenyl]-2-indolinone (Structure 3), 4-methyl-5-(2-oxo-1,2-dihydroindol-3-ylidenemethyl)-1H-pyrrole-2-carboxylic acid (Structure 4), 4-methyl-5-(2-oxo-1,2-dihydroindol-3-ylidenemethyl)-1H-pyrrole-2-carboxylic acid methyl ester (Structure 5), 3-(5-hydroxymethyl-3-methyl-1H-pyrrol-2-ylmethylene)-1,3-dihydroindole-2-one (Structure 6) and 4-methyl-5-(2-oxo-1,2-dihydroindol-3-ylidenemethyl)-1H-pyrrole-2-carbaldehyde (Structure 7).

Physiologically acceptable salts and prodrugs of the above compounds are within the scope of this invention.

In another aspect, this present invention relates to a method of modulating the catalytic activity of PKs comprising contacting the PK with a compound having one of the structures shown above.

As used herein, the term “modulation” or “modulating” refers to the alteration of the catalytic activity of RTKs, CTKs and STKs. In particular, modulating refers to the activation of the catalytic activity of RTKs, CTKs and STKs, preferably the activation or inhibition of the catalytic activity of RTKS, CTKs and STKS, depending on the concentration of the compound or salt to which the RTK, CTK or STK is exposed or, more preferably, the inhibition of the catalytic activity of RTKs, CTKs and STKs.

The term “catalytic activity” as used herein refers to the rate of phosphorylation of tyrosine under the influence, direct or indirect, of RTKs and/or CTKs or the phosphorylation of serine and threonine under the influence, direct or indirect, of STKs.

The term “contacting” as used herein refers to bringing a compound of this invention and a target PK together in such a manner that the compound can affect the catalytic activity of the PK, either directly; i.e., by interacting with the kinase itself, or indirectly; i.e., by interacting with another molecule on which the catalytic activity of the kinase is dependent. Such “contacting” can be accomplished “in vitro,” i.e., in a test tube, a petri dish or the like. In a test tube, contacting may involve only a compound and a PK of interest or it may involve whole cells. Cells may also be maintained or grown in cell culture dishes and contacted with a compound in that environment. In this context, the ability of a particular compound to affect a PK related disorder; i.e., the IC₅₀ of the compound, defined below, can be determined before use of the compounds in vivo with more complex living organisms is attempted. For cells outside the organism, multiple methods exist, and are well-known to those skilled in the art, to get the PKs in contact with the compounds including, but not limited to, direct cell microinjection and numerous transmembrane carrier techniques.

The above-referenced PK is selected from the group consisting of an RTK, a CTK or an STK in another aspect of this invention.

Furthermore, it is an aspect of this invention that the receptor protein kinase whose catalytic activity is modulated by a compound of this invention is selected from the group consisting of EGF, HER2, HER3, HER4, IR, IGF-1R, IRR, PDGFRα, PDGFRβ, CSFIR, C-Kit, C-fms, Flk-1R, Flk4, KDR/Flk-1, Flt-1, FGFR-1R, FGFR-2R, FGFR-3R and FGFR-4R.

In addition, it is an aspect of this invention that the cellular tyrosine kinase whose catalytic activity is modulated by a compound of this invention is selected from the group consisting of Src, Frk, Btk, Csk, Abl, ZAP70, Fes, Fps, Fak, Jak, Ack, Yes, Fyn, Lyn, Lck, Blk, Hck, Fgr and Yrk.

Another aspect of this invention is that the serine-threonine protein kinase whose catalytic activity is modulated by a compound of this invention is selected from the group consisting of CDK2 and Raf.

In another aspect, this invention relates to a method for treating or preventing a PK-related disorder in a patient in need of such treatment comprising administering to the patient a therapeutically effective amount of one or more of the compounds described above.

As used herein, “PK related disorder,” “PK driven disorder,” and “abnormal PK activity” all refer to a condition characterized by inappropriate; i.e., under or, more commonly, over, PK catalytic activity, where the particular PK can be an RTK, a CTK or an STK. Inappropriate catalytic activity can arise as the result of either: (1) PK expression in cells which normally do not express PKs; (2) increased PK expression leading to unwanted cell proliferation, differentiation and/or growth; or, (3) decreased PK expression leading to unwanted reductions in cell proliferation, differentiation and/or growth. Over-activity of a PK refers to either amplification of the gene encoding a particular PK or production of a level of PK activity which can correlate with a cell proliferation, differentiation and/or growth disorder (that is, as the level of the PK increases, the severity of one or more of the symptoms of the cellular disorder increases). Under-activity is, of course, the converse, wherein the severity of one or more symptoms of a cellular disorder increase as the level of the PK activity decreases.

“Treat,” “treating” or “treatment” with regard to a PK-related disorder refers to alleviating or abrogating the cause and/or the effects of a PK-related disorder.

As used herein, the terms “prevent”, “preventing” and “prevention” refer to a method for barring an organism from acquiring a PK related disorder in the first place.

The PK related disorder may be selected from the group consisting of an RTK, a CTK and an STK related disorder in a further aspect of this invention.

In yet another aspect of this invention, the above referenced PK related disorder may be selected from the group consisting of an EGFR related disorder, a PDGFR related disorder, an IGFR related disorder and a flk related disorder.

The above referenced protein kinase related disorder is a cancer selected from the group consisting of squamous cell carcinoma, astrocytoma, glioblastoma, lung cancer, bladder cancer, head and neck cancer, melanoma, ovarian cancer, prostate cancer, breast cancer, small-cell lung cancer, colorectal cancer, gastrointestinal cancer and glioma in a further aspect of this invention.

The above referenced protein kinase related disorder is selected from the group consisting of diabetes, an autoimmune disorder, a hyperproliferation disorder, restenosis, fibrosis, psoriasis, osteoarthritis, rheumatoid arthritis, an inflammatory disorder and angiogenesis in yet another aspect of this invention.

Other disorders which might be treated with compounds of this invention include, without limitation, immunological and cardiolovascular disorders such as, for instance aetherosclerosis.

Pharmaceutical compositions of the above compounds are a further aspect of this invention.

A “pharmaceutical composition” refers to a mixture of one or more of the compounds or drugs described herein, or physiologically acceptable salts or prodrugs thereof, with other chemical components, such as physiologically acceptable carriers and excipients. The purpose of a pharmaceutical composition is to facilitate administration of a compound to an organism.

As used herein, a “physiologically acceptable carrier” refers to a carrier or diluent that does not abrogate the biological activity and properties of the administered compound while facilitating administration by, for example, stabilizing or solubilizing the compound. Preferably, the carrier does not cause significant irritation to the organism.

An “excipient” refers to a substance added to a pharmaceutical composition to further facilitate administration of a compound. Examples, without limitation, of excipients include calcium carbonate, calcium phosphate, various sugars and types of starch, cellulose derivatives, gelatin, vegetable oils, surfactants and polyethylene glycols.

Yet another aspect of this invention is a method for inhibiting tumorigenic activity in a cell comprising contacting the cell with a 3-heteroarylidenyl-2-indolinone of this invention.

“Tumorigenic” activity, as used herein and as it relates to a cell, refers to both intracellular and extracellular biochemical activity which contributes to the formation of a neoplasm.

A “neoplasm” is an abnormal tissue that grows by cellular proliferation more rapidly than normal and continues to grow even after the stimuli that initiated the new growth cease. A neoplasm partially or completely lacks structural organization and functional coordination with the normal tissue and usually forms a distinct mass of tissue. Such masses may be benign (benign tumors) or malignant (solid tumor cancer). Malignant neoplasms are locally invasive and destructive and in many cases metastasize (spread to and invade and destroy tissues in areas of the affected organism remote from the site of origin). The process of neoplasm formation is generally referred to as “neoplasia”; i.e. neoplasia is the biochemical process by which a neoplasm forms and grows.

The terms “malignant neoplasm”, “cancer”, “tumor” and “solid tumor cancer” are used interchangeably herein to refer to the condition well known to those skilled in the art as the life-threatening disease commonly referred to simply as “cancer”.

With regard to tumorigenic activity, “inhibit” or “inhibiting” refers to eliminating, reducing, containing, impeding, preventing, slowing, retarding and/or restricting neoplasia.

A “chemotherapeutic agent” refers to a chemical substance or drug used to treat a disease; the term is most often applied to such substances or drugs which are used primarily for the treatment of cancer.

### DETAILED DESCRIPTION OF THE INVENTION

1. Indications/Target Diseases

General

The PKs whose catalytic activity is modulated by the compounds of this invention include protein tyrosine kinases of which there are two types, receptor tyrosine kinases (RTKs) and cellular tyrosine kinases (CTKs), and serine-threonine kinases (STKs). RTK mediated signal transduction, is initiated by extracellular interaction with a specific growth factor (ligand), followed by receptor dimerization, transient stimulation of the intrinsic protein tyrosine kinase activity and phosphorylation. Binding sites are thereby created for intracellular signal transduction molecules and lead to the formation of complexes with a spectrum of cytoplasmic signaling molecules that facilitate the appropriate cellular response (e.g., cell division, metabolic effects on the extracellular microenvironment, etc.). See, Schlessinger and Ullrich, 1992, Neuron 9:303-391.

It has been shown that tyrosine phosphorylation sites on growth factor receptors function as high-affinity binding sites for SH2 (src homology) domains of signaling molecules. Fantl et al., 1992, Cell 69:413-423; Songyang et al., 1994, Mol. Cell. Biol. 14:2777-2785); Songyang et al., 1993, Cell 72:767-778; and Koch et al., 1991, Science 252:668-678. Several intracellular substrate proteins that associate with RTKs have been identified. They may be divided into two principal groups: (1) substrates which have a catalytic domain; and (2) substrates which lack such domain but which serve as adapters and associate with catalytically active molecules. Songyang et al., 1993, Cell 72:767-778. The specificity of the interactions between receptors and SH2 domains of their substrates is determined by the amino acid residues immediately surrounding the phosphorylated tyrosine residue. Differences in the binding affinities between SH2 domains and the amino acid sequences surrounding the phosphotyrosine residues on particular receptors are consistent with the observed differences in their substrate phosphorylation profiles. Songyang et al., 1993, Cell 72:767-778. These observations suggest that the function of each RTK is determined not only by its pattern of expression and ligand availability but also by the array of downstream signal transduction pathways that are activated by a particular receptor. Thus, phosphorylation provides an important regulatory step which determines the selectivity of signaling pathways recruited by specific growth factor receptors, as well as differentiation factor receptors.

STKs, being primarily cytosolic, affect the internal biochemistry of the cell, often as a down-line response to a PTK event. STKs have been implicated in the signaling process which initiates DNA synthesis and subsequent mitosis leading to cell proliferation.

Thus, PK signal transduction results in, among other responses, cell proliferation, differentiation, growth and metabolism. Abnormal cell proliferation may result in a wide array of disorders and diseases, including the development of neoplasia such as carcinoma, sarcoma, glioblastoma and hemangioma, disorders such as leukemia, psoriasis, arteriosclerosis, arthritis and diabetic retinopathy and other disorders related to uncontrolled angiogenesis and/or vasculogenesis.

A precise understanding of the mechanism by which the compounds of this invention inhibit PKs is not required in order to practice the present invention. However, while not hereby being bound to any particular mechanism or theory, it is believed that the compounds interact with the amino acids in the catalytic region of PKs. PKs typically possess a bi-lobate structure wherein ATP appears to bind in the cleft between the two lobes in a region where the amino acids are conserved among PKs. Inhibitors of PKs are believed to bind by non-covalent interactions such as hydrogen bonding, van der Waals forces and ionic interactions in the same general region where the aforesaid ATP binds to the PKs. More specifically, it is thought that the 2-indolinone component of the compounds of this invention binds in the general space normally occupied by the adenine ring of ATP. Specificity of a particular molecule for a particular PK may then arise as the result of additional interactions between the various substituents on the 2-indolinone core and the amino acid domains specific to particular PKs. Thus, different indolinone substituents may contribute to preferential binding to particular PKs. The ability to select compounds active at different ATP (or other nucleotide) binding sites makes the compounds of this invention useful for targeting any protein with such a site. The compounds disclosed herein may thus have utility as in vitro assays for such proteins as well as exhibiting in vivo therapeutic effects through interaction with such proteins.

In another aspect, the protein kinase, the catalytic activity of which is modulated by contact with a compound of this invention, is a protein tyrosine kinase, more particularly, a receptor protein tyrosine kinase. Among the receptor protein tyrosine kinases whose catalytic activity can be modulated with a compound of this invention, or salt thereof, are, without limitation, EGF, HER2, HER3, HER4, IR, IGF-1R, IRR, PDGFRα, PDGFRβ, CSFIR, C-Kit, C-fms, Flk-1R, Flk4, KDR/Flk-1, Flt-1, FGFR-1R, FGFR-2R, FGFR-3R and FGFR-4R.

The protein tyrosine kinase whose catalytic activity is modulated by contact with a compound of this invention, or a salt or a prodrug thereof, can also be a non-receptor or cellular protein tyrosine kinase (CTK). Thus, the catalytic activity of CTKs such as, without limitation, Src, Frk, Btk, Csk, Abl, ZAP70, Fes, Fps, Fak, Jak, Ack, Yes, Fyn, Lyn, Lck, Blk, Hck, Fgr and Yrk, may be modulated by contact with a compound or salt of this invention.

Still another group of PKs which may have their catalytic activity modulated by contact with a compound of this invention are the serine-threonine protein kinases such as, without limitation, CDK2 and Raf.

In another aspect, this invention relates to a method for treating or preventing a PK related disorder by administering a therapeutically effective amount of a compound of this invention, or a salt or a prodrug thereof, to an organism.

It is also an aspect of this invention that a pharmaceutical composition containing a compound of this invention or a salt or prodrug thereof is administered to an organism for the purpose of preventing or treating a PK related disorder.

This invention is therefore directed to compounds which modulate PK signal transduction by affecting the enzymatic activity of RTKs, CTKs and/or STKs, thereby interfering with the signals transduced by such proteins. More particularly, the present invention is directed to compounds which modulate RTK, CTK and/or STK mediated signal transduction pathways as a therapeutic approach to cure many kinds of solid tumors, including but not limited to carcinomas, sarcomas including Kaposi's sarcoma, erythroblastoma, glioblastoma, meningioma, astrocytoma, melanoma and myoblastoma. Treatment or prevention of non-solid tumor cancers such as leukemia are also contemplated by this invention. Indications may include, but are not limited to brain cancers, bladder cancers, ovarian cancers, gastric cancers, pancreatic cancers, colon cancers, blood cancers, lung cancers and bone cancers.

Further examples, without limitation, of the types of disorders related to inappropriate PK activity that the compounds described herein may be useful in preventing, treating and studying, are cell proliferative disorders, fibrotic disorders and metabolic disorders.

Cell proliferative disorders, which may be prevented, treated or further studied by the present invention include cancer, blood vessel proliferative disorders and mesangial cell proliferative disorders.

Blood vessel proliferative disorders refer to disorders related to abnormal vasculogenesis (blood vessel formation) and angiogenesis (spreading of blood vessels). While vasculogenesis and angiogenesis play important roles in a variety of normal physiological processes such as embryonic development, corpus luteum formation, wound healing and organ regeneration, they also play a pivotal role in cancer development where they result in the formation of new capillaries needed to keep a tumor alive. Other examples of blood vessel proliferation disorders include arthritis, where new capillary blood vessels invade the joint and destroy cartilage, and ocular diseases, like diabetic retinopathy, where new capillaries in the retina invade the vitreous, bleed and cause blindness.

Conversely, disorders related to the shrinkage, contraction or closing of blood vessels, such as restenosis, are also implicated and may be treated or prevented by the methods of this invention.

Fibrotic disorders refer to the abnormal formation of extracellular matrices. Examples of fibrotic disorders include hepatic cirrhosis and mesangial cell proliferative disorders. Hepatic cirrhosis is characterized by the increase in extracellular matrix constituents resulting in the formation of a hepatic scar. An increased extracellular matrix resulting in a hepatic scar can also be caused by a viral infection such as hepatitis. Lipocytes appear to play a major role in hepatic cirrhosis. Other fibrotic disorders implicated include atherosclerosis.

Mesangial cell proliferative disorders refer to disorders brought about by abnormal proliferation of mesangial cells. Mesangial proliferative disorders include various human renal diseases such as glomerulonephritis, diabetic nephropathy and malignant nephrosclerosis as well as such disorders as thrombotic microangiopathy syndromes, transplant rejection, and glomerulopathies. The RTK PDGFR has been implicated in the maintenance of mesangial cell proliferation. Floege et al., 1993, Kidney International, 43:47S-54S.

Many cancers are cell proliferative disorders and, as noted previously, PKs have been associated with cell proliferative disorders. Thus, it is not surprising that PKs such as, for example, members of the RTK family have been associated with the development of cancer. Some of these receptors, like EGFR (Tuzi et al., Br. J. Cancer, 1992, 63:227-233; Torp et al., 1992, APMIS 100:713-719) HER2/neu (Slamon et al., Science, 1989, 244:707-712) and PDGF-R (Kumabe et al., Oncogene, 1992, 7:627-633) are over-expressed in many tumors and/or persistently activated by autocrine loops. In fact, in the most common and severe cancers these receptor over-expressions (Akbasak and Suner-Akbasak et al., J. Neurol. Sci., 1992, 111:119-133; Dickson et al., Cancer Treatment Res., 1992, 61:249-273; Korc et al., J. Clin. Invest., 1992, 90:1352-1360) and autocrine loops (Lee and Donoghue, J. Cell. Biol., 1992, 118:1057-1070; Korc et al., supra; Akbasak and Suner-Akbasak et al., supra) have been demonstrated. For example, EGFR has been associated with squamous cell carcinoma, astrocytoma, glioblastoma, head and neck cancer, lung cancer and bladder cancer. HER2 has been associated with breast, ovarian, gastric, lung, pancreas and bladder cancer. PDGFR has been associated with glioblastoma and melanoma as well as lung, ovarian and prostate cancer. The RTK c-met has also been associated with malignant tumor formation. For example, c-met has been associated with, among other cancers, colorectal, thyroid, pancreatic, gastric and hepatocellular carcinomas and lymphomas. Additionally c-met has been linked to leukemia. Over-expression of the c-met gene has also been detected in patients with Hodgkins disease and Burkitts disease.

Flk has likewise been associated with a broad spectrum of tumors including, without limitation, mammary, ovarian and lung tumors as well as gliomas such as glioblastoma.

IGF-IR, in addition to being implicated in nutritional support and in type-II diabetes, has also been associated with several types of cancers. For example, IGF-I has been implicated as an autocrine growth stimulator for several tumor types, e.g. human breast cancer carcinoma cells (Arteaga et al., J. Clin. Invest., 1989, 84:1418-1423) and small lung tumor cells (Macauley et al., Cancer Res., 1989, 50:2511-2517). In addition, IGF-I, while integrally involved in the normal growth and differentiation of the nervous system, also appears to be an autocrine stimulator of human gliomas. Sandberg-Nordqvist et al., Cancer Res., 1993, 53:2475-2478. The importance of IGF-IR and its ligands in cell proliferation is further supported by the fact that many cell types in culture (fibroblasts, epithelial cells, smooth muscle cells, T-lymphocytes, myeloid cells, chondrocytes and osteoblasts (the stem cells of the bone marrow)) are stimulated to grow by IGF-I. Goldring and Goldring, Eukaryotic Gene Expression, 1991, 1:301-326. In a series of recent publications, Baserga suggests that IGF-IR plays a central role in the mechanism of transformation and, as such, could be a preferred target for therapeutic interventions for a broad spectrum of human malignancies. Baserga, Cancer Res., 1995, 55:249-252;

Baserga, Cell, 1994, 79:927-930; Coppola et al., Mol. Cell. Biol., 1994, 14:4588-4595.

STKs have been implicated in many types of cancer including, notably, breast cancer (Cance, et al., Int. J. Cancer, 1993, 54:571-77).

The association between abnormal PK activity and disease is not restricted to cancer. For example, RTKs have been associated with diseases such as psoriasis, diabetes mellitus, endometriosis, angiogenesis, atheromatous plaque development, Alzheimer's disease, epidermal hyperproliferation, neurodegenerative diseases, age-related macular degeneration and hemangiomas. For example, EGFR has been indicated in corneal and dermal wound healing. Defects in Insulin-R and IGF-1R are indicated in type-II diabetes mellitus. A more complete correlation between specific RTKs and their therapeutic indications is set forth in Plowman et al., DN&P, 1994, 7:334-339.

As noted previously, not only RTKs but CTKs including, but not limited to, src, abl, fps, yes, fyn, lyn, lck, blk, hck, fgr and yrk (reviewed by Bolen et al., FASEB J., 1993, 6:3403-3409) are involved in the proliferative and metabolic signal transduction pathway and thus could be expected, and have been shown, to be involved in many PTK-mediated disorders to which the present invention is directed. For example, mutated src (v-src) has been shown to be an oncoprotein (pp₆₀^{v-src}) in chicken. Moreover, its cellular homolog, the proto-oncogene pp60^{c-src} transmits oncogenic signals of many receptors. Over-expression of EGFR or HER2/neu in tumors leads to the constitutive activation of pp60^{c-src}, which is characteristic of malignant cells but absent in normal cells. On the other hand, mice deficient in the expression of c-src exhibit an osteopetrotic phenotype, indicating a key participation of c-src in osteoclast function and a possible involvement in related disorders.

Similarly, Zap70 has been implicated in T-cell signaling which may relate to autoimmune disorders.

STKs have been associated with inflamation, autoimmune disease, immunoresponses, and hyperproliferation disorders such as restenosis, fibrosis, psoriasis, osteoarthritis and rheumatoid arthritis.

PKs have also been implicated in embryo implantation. Thus, the compounds of this invention may provide an effective method of preventing such embryo implantation and thereby be useful as birth control agents.

Finally, both RTKs and CTKs are currently suspected as being involved in hyperimmune disorders.

A method for identifying a chemical compound that modulates the catalytic activity of one or more of the above discussed protein kinases is another aspect of this invention. The method involves contacting cells expressing the desired protein kinase with a compound of this invention (or its salt or prodrug) and monitoring the cells for any effect that the compound has on them. The effect may be any observable, either to the naked eye or through the use of instrumentation, change or absence of change in a cell phenotype. The change or absence of change in the cell phenotype monitored may be, for example, without limitation, a change or absence of change in the catalytic activity of the protein kinase in the cells or a change or absence of change in the interaction of the protein kinase with a natural binding partner.

VEGF and Flk-1/KDR in Angiogenesis and Colorectal Cancer

Tumor cells stimulate quiescent endothelial cells to divide and form new blood vessels by releasing growth factors, which bind to nearby endothelial cells (a paracrine mode of action). Binding of vascular endothelial growth factor (“VEGF”) to one of its receptors begins the signaling cascade that regulates cellular events involved in new blood vessel formation.

A number of receptor tyrosine kinases are thought to be directly or indirectly involved in angiogenesis. The search for the receptor whose selective inhibition will prevent new blood vessel growth to support growing tumors has been the focus of basic research for the last ten years. Although there are multiple receptors whose expression is restricted to endothelial cells (including Flk-1, Flt-1, Tie-1 and Tie-2), it is believed that the Flk-1 receptor plays a critical role in angiogenesis.

The temporal and spatial patterns of expression of VEGF and its receptors support a role for these in normal angiogenesis during embryonic development. VEGF, Flt-1 and Flk-1 have also been implicated in pathological angiogenesis to support the growth of many solid tumors, including gliomas, breast cancer, bladder cancer, colon carcinoma and other gastrointestinal tract cancers. A correlation has been observed between VEGF expression and vessel density in breast tumors, renal cell carcinoma and colon cancer. In highly vascularized glioblastoma, transcripts for VEGF and its receptors were identified by in situ hybridization; transcripts were not detected in the less vascular, low grade gliomas or in normal brain tissue. In this setting (supporting a paracrine mode of action), Flk-1 receptors were detected in the endothelial cells of the vessels while VEGF localized to the tumor cells. Expression of VEGF and its receptors has been shown for hematopoietic tumor cell lines including multiple myeloma.

VEGF is mitogenic for endothelial cells in vitro. In such a system, neutralizing antibodies against Flk-1 inhibit mitogenesis. Similarly, ribozymes that cleave flk-1 or flt-1 mRNAs reduce the growth of human microvasculature endothelial cells, presumably by decreasing the number of receptors on the cells.

A variety of in vivo techniques have been used to investigate the role of VEGF signaling in tumor angiogenesis. Flk-1 receptors which lack the intracellular kinase domain block the activation of the endogenous Flk-1 receptor activity in cultured cells, inhibiting the growth of tumors implanted subcutaneously into nude mice. Any tumors that did form in this animal model contained significantly reduced vessel density. Also, reduction in VEGF expression with antisense constructs inhibits the growth of C6 rat glioma cells in nude mice with concurrent reduced blood vessel density in these tumors and inhibits the growth of human melanoma cells in nude/SCID mice. Likewise, reduction of VEGF levels with neutralizing antibodies inhibits the growth of human rhabdomyosarcoma, glioblastoma multiforme and leiomyosarcoma in nude mice and fibrosarcoma in BALBc/nude mice.

Taken together, these results provide strong evidence for a critical role of VEGF signaling through Flk-1 in angiogenesis in solid tumor growth. An inhibitor of Flk-1 may have therapeutic benefit in cancer patients.

2. Pharmacology

Preclinical Studies with 3-[(2,4-dimethylpyrrol-5-yl)methylidenyl]-2-indolinone

In a cellular-based assay, 3-[(2,4-dimethylpyrrol-5-yl)methylidenyl]-2-indolinone has been found to inhibit the receptor phosphorylation that typically follows the interaction of VEGF with its receptor. In vitro studies of 3-[(2,4-dimethylpyrrol-5-yl)methylidenyl]-2-indolinone have demonstrated its ability to inhibit Flk-1 autophosphorylation with IC₅₀ values of approximately 1 μM. In addition, 3-[(2,4-dimethylpyrrol-5-yl)methylidenyl]-2-indolinone inhibits in vitro proliferation of endothelial cells induced by VEGF with IC₅₀ values of approximately 0.07 μM. In this assay, 3-[(2,4-dimethylpyrrol-5-yl)methylidenyl]-2-indolinone exerts a time-dependent increase in potency, with detectable activity first observed after a 5-minute exposure to drug. One-hour exposure to 3-[(2,4-dimethylpyrrol-5-yl)methylidenyl]-2-indolinone results in in vitro antiproliferative activity for 3 to 4 days thereafter. 3-[(2,4-dimethylpyrrol-5-yl)methylidenyl]-2-indolinone has no direct inhibitory effects on a variety of tumor cell lines at concentrations up to 50 μM.

In vivo studies of 3-[(2,4-dimethylpyrrol-5-yl)methylidenyl]-2-indolinone, in which a variety of tumor cell lines were subcutaneously implanted into immunocompromised mice, 3-[(2,4-dimethylpyrrol-5-yl)methylidenyl]-2-indolinone demonstrates a significant suppression of tumor growth against a broad spectrum of tumor types whose growth are driven by various growth factors such as PDGF, EGF and Her2. Daily intraperitoneal dosing (ranging from 12.5-25 mg/kg/day for 28 days) resulted in 30-80% inhibition of tumor growth. In initial studies, 3-[(2,4-dimethylpyrrol-5-yl)methylidenyl]-2-indolinone administration was started on Day 1 after tumor implantation. Later studies, in which 3-[(2,4-dimethylpyrrol -5-yl)methylidenyl]-2-indolinone administration was delayed until tumors were grown to a volume of approximately 50 mm³, demonstrated equivalent efficacy in suppression of tumor growth.

Dose response studies with 3-[(2,4-dimethylpyrrol-5-yl)methylidenyl]-2-indolinone (at doses between 6.25-25 mg/kg/day) were conducted with human melanoma cells implanted subcutaneously in athymic mice. Daily administration of 3-[(2,4-dimethylpyrrol-5-yl)methylidenyl]-2-indolinone at doses as low as 1 mg/kg/day resulted in dose-dependent inhibition of these cells. Additional studies with intraperitoneal dosing of 3-[(2,4-dimethylpyrrol-5-yl)methylidenyl]-2-indolinone in athymic mice using less frequent administration (including twice weekly for four weeks) also resulted in equivalent tumor growth inhibition when compared to daily intraperitoneal administration (77% in twice weekly dosing versus 68% with daily dosing).

Daily administration of 3-[(2,4-dimethylpyrrol-5-yl)methylidenyl]-2-indolinone (25 mg/kg/day) was also shown to significantly inhibit the growth of tumor cells surgically implanted under the serosa of the colon. Treatment with 3-[(2,4-dimethylpyrrol-5-yl)methylidenyl]-2-indolinone leads to both decreased tumor size and decreased vascularization, as evidenced by the pale appearance of tumors in 3-[(2,4-dimethylpyrrol-5yl)methylidenyl]2-indolinone-treated animals.

Pharmacokinetics of 3-[(2,4-dimethylpyrrol-5-yl)methyl-idenyl]-2-indolinone.

Washout experiments in vitro have indicated a target half-life of 96 hours, suggesting a very tight competitive binding of 3-[(2,4-dimethylpyrrol-5-yl)methylidenyl]-2-indolinone to the ATP binding site of the receptor tyrosine kinase. The in vivo intravenous pharmacokinetics of 3-[(2,4-dimethylpyrrol-5-yl)methylidenyl]-2-indolinone was characterized by rapid elimination of the parent compound from the circulation in mice, rats and dogs. There was a slightly longer elimination half-life determined for the rat in comparison to mice and dogs. (Data not shown).

Pharmacokinetics of 3-[(2,4-dimethylpyrrol-5-yl)methylidenyl]-2-indolinone in rats are dose-dependent at higher intravenous doses. At doses between 29.5-97.9 mg/m², the elimination half-life of 3-[(2,4-dimethylpyrrol-5-yl)methylidenyl]-2-indolinone linearly increases as the dose increased; the AUC increases 10-fold with only a 3-fold increase in dose.

Subchronic toxicokinetic studies (28 day toxicity studies) in rats and dogs indicated that the drug did not accumulate in plasma upon repeated administration.

Whole body autoradiography using [¹⁴C]-3-[(2,4-dimethylpyrrol-5-yl)methylidenyl]-2-indolinone demonstrated widespread tissue distribution of 3-[(2,4-dimethylpyrrol-5-yl)methylidenyl]-2-indolinone followed by rapid elimination following intravenous injection, with the highest levels present in the small intestinal contents and urine (with additional 3-[(2,4-dimethylpyrrol-5-yl)methylidenyl]-2-indolinone observed in the liver, kidney, skin, testis, brown fat, harderian gland and nasal turbinates). Total dose recovered in 24 hours equaled 92% of the total administered dose, with 72% excreted in feces and 16% excreted in urine. Biliary excretion is thought to be the major route of elimination.

Studies with cold and [¹⁴C]-labeled 3-[(2,4-dimethylpyrrol-5-yl)methylidenyl]-2-indolinone demonstrate that the compound is rapidly metabolized following intravenous administration in rats. Radiometabolite profiling indicated that greater than 90% of [¹⁴C]-3-[(2,4-dimethylpyrrol-5-yl)methylidenyl]-2-indolinone was metabolized within 3 hours following intravenous administration. Data on metabolite identification suggest that one metabolite has added a carboxyl group to one of the methyl groups on the pyrrole ring, with a second metabolite adding a methyl to the carboxyl group.

Preliminary pharmacokinetic data from a Phase 1 study in patients with advanced malignancies in which patients were treated at doses between 4.4-190 mg/m² indicates that the drug has a half-life in humans of approximately 60 minutes. The alpha half-life is rapid, with a mean 5.8±1.9 minutes. The beta half-life or elimination phase has a mean value 43.4±21.9 minutes with a range from 10-111 minutes. Clearance of 3-[(2,4-dimethylpyrrol-5-yl)methylidenyl]-2-indolinone from the systemic circulation is rapid, with a mean value 1857±1016 liters of plasma cleared of 3-[(2,4-dimethylpyrrol-5-yl)methylidenyl]-2-indolinone per day. Clearance was independent of dose at these levels. Individual clearance calculated based on BSA equaled 41.8±22.1 L/hour/m2. After eight infusions of 3-[(2,4-dimethylpyrrol-5-yl)methylidenyl]-2-indolinone, the rate of clearance increases by 50-300% in all patients. The total distributive volume of 3-[(2,4-dimethylpyrrol-5-yl)methylidenyl]-2-indolinone, calculated by a one-compartment model, is 53.6±11.3 liters, indicating that the drug is distributed in the whole body fluid. At doses tested in humans to date, AUC and C_{MAX} increase linearly with dose.

The primary pathway for metabolism of 3-[(2,4-dimethylpyrrol-5-yl)methylidenyl]-2-indolinone is through sequential oxidation reactions of the 5-methyl group on the pyrrol ring. Four metabolites are measurable in serum, all of which involve serial oxidations of this methyl group on the pyrrol ring. Data from in vitro metabolism studies shows 3-[(2,4-dimethylpyrrol-5-yl)methylidenyl]-2-indolinone is metabolized via β-450 liver enzymes, most probably via CYP1A2 and CYP3A4, both of which are inducible enzymes. In particular, CYP3A4 is induced by many xenobiotics, including dexamethasone which is administered as a premedication prior to all 3-[(2,4-dimethylpyrrol-5-yl)methylidenyl]-2-indolinone injections.

Fluorouracil and Fluorouracil/Leucovorin—General

The chemical structure of fluorouracil is 5-fluoro-2,4 (1H,3H)-pyrimidinedione. While the precise mode of action of fluorouracil is not clear, the drug is thought to function as an antimetabolite in at least three ways. In one aspect, as its deoxyribonucleotide derivative, 5-fluoro-2′-deoxyuridine-5′-phosphate (F-dUMP), the drug inhibits thymidylate synthetase which results in inhibition of methylation of deoxyuridylic acid to thymidylic acid. This, in turn, interferes with the synthesis of DNA. In a second aspect, fluorouracil is found to be incorporated into RNA to a an extent which, although small, is sufficient to have a major effect on both the processing and functions of the RNA. Finally, in a third aspect, fluorouracil has been shown to block uracil phosphatase thus inhibiting utilization of preformed uracil in RNA synthesis (Goodman and Gilman's, “The Pharmacological Basis of Therapeutics”, 1985, pages 1268-1271).

Fluorouracil can be administered alone or in combination with other drugs. The most common combination involves the use of leucovorin (folinic acid). Leucovorin potentiates the cytotoxic effect of fluorouracil by, it is thought, increasing the extracellular concentration of reduced folates which in turn appears to stabilize the covalent ternary complex formed by (F-dUMP), 5,10-methylenetetrahydrofolate and thymidine synthetase. The stabilization of this complex enhances inhibition of the synthetase, thereby increasing the efficacy of fluorouracil.

Other chemotherapeutic combinations with fluorouracil for the treatment of advanced stage colorectal cancer which have been utilized include, without limitation, combination of fluorouracil with: methotrexate, alone (Blijham, G., et al., J. Clin Oncol., 1996, 14(8):2266-73) and in combination with leucovorin (Romero, A. O., et al., Am. J. Clin. Onocol., 1998, 21(1):94-8); interferon alfa-2a (Greco, F. A., et al., J. Clin. Oncol., 1996, 14(10):2674-81); interferon alpha 2b plus leucovorin (Kohne, C. H., Oncology, 1997, 54(2):96-101): platinum compounds, such as cisplatin and oxaliplatin, in combination with leucovorin (Scheithauer, W., et al., Cancer, 1994, 73(6):1562-68); carboplatin plus methotrexate (prior to fluorouracil administration) (Pronzato, P., J. Chemother., 1998, 10(3):254-57); and Bleiberg, H. and Gramont, A., Semin. Oncol., 1998, 25(2 Suppl. 5):32-39): lavamisole (Bandealy, M. T., Clin. Cancer Res., 1998, 4(4):935-38); methyl lomustine and leucovorin (Jones, Jr., D. V., Cancer, 1995, 76(10):1709-14); and, irinotecan, a topoisomerase-I inhibitor, (after pretreatment with fluorouracil/leucovorin) (Rougier, P. et al., J. Clin. Oncol., 1997, 15(1):251-260).

While use of the above combinations is increasing, none of them at present appear to provide a clear advantage over fluorouracil alone or fluorouracil in combination with leucovorin; that latter remains the standard initial treatment for patients with metastatic colorectal cancer. As a single agent, it produces response rates of about 15% with a median survival of six months. In combination with leucovorin, the activity of the fluorouracil is increased such that response rates of about 20% and median survival times in advanced (Stage D) colorectal cancer of 11-13 months are observed (Wolmark, N., et al., J. Clin. Oncol., 1993, 11:1879-1887). Fluorouracil may be adminstered by either intravenous bolus injection or continuous infusion. The volume of distribution is slightly larger than the extracellular space.

Intravenous bolus doses of 370 to 720 mg/m² produce an elimination half-life of 8 to 14 minutes with plasma levels below 1 μM within 2 hours, an approximate threshold for cytotoxic effects. Less than 10% of the drug is excreted in urine, while the balance is cleared through metabolic pathways.

Frequently used administration schedules include short-bolus injections over three to five days every 3-4 weeks, continuous intravenous infusions of 96-120 hour duration every 4 weeks, and weekly infusions for six weeks out of every eight weeks. The incidence of serious clinical toxicity tends to increase with higher systemic exposure (for example, with higher steady-state plasma concentrations during constant infusions and higher AUC with bolus administration).

Notably, each of the above schedules of treatment includes substantial intervals during which no fluorouracil is administered. This is due primarily to the inherent toxicity of fluorouracil, which is exacerbated by the addition of leucovorin. Unfortunately, this time interval substantially reduces the efficacy of fluorouracil. That is, initial treatment of a patient with fluorouracil or fluorouracil/leucovorin produces about a three log unit (three orders of magnitude or 1000-fold) reduction in tumor number and size. However, during the no-treatment “recovery” period, tumor number and size rebound to the extent of about two log units (100-fold). Thus, the overall effect of a course of treatment with fluorouracil is only about one log unit (an approximately 10-fold decrease in tumor number and size) per administration of fluorouracil. Not only does prolonged treatment with fluorouracil cause a problem with regard to cost of treatment, patient quality of life, etc., it can result in secondary resistance to the drug. The methods of this invention are directed to maintaining a more substantial portion of the effect of each administration of fluorouracil during the recovery period. Subsequent administrations in the full course of treatment will thus be confronted with tumors of reduced size and number, thus improving the overall effectiveness of fluorouracil.

Clinical Trials with Fluorouracil and Fluorouracil/Leucovorin in Advanced Colorectal Cancer

Frequently used continuous infusion schedules include short-bolus injections over three to five days every 3-4 weeks, continuous intravenous infusions of 96-120 hours every 4 weeks, and weekly infusions for six weeks out of every eight weeks. The incidence of serious clinical toxicity tends to increase with higher systemic exposure (for example, with higher steady-state plasma concentrations during constant infusions and higher AUC with bolus administration).

In a randomized clinical study conducted by the Mayo Clinic and the North Central Cancer Treatment Group (Mayo/NCCTG) in patients with advanced metastatic colorectal cancer, three treatment regimens were compared: Leucovorin (leucovorin) 200 mg/m² and fluorouracil 370 mg/M² versus leucovorin 20 mg/m² and fluorouracil 425 mg/m² versus fluorouracil 500 mg/m². All drugs were administered by slow intravenous infusion daily for 5 days repeated every 28-35 days. Response rates were 26% (p=0.04 versus fluorouracil alone), 43% (p=0.001 versus fluorouracil alone) and 10% for the high dose leucovorin, low dose leucovorin and fluorouracil alone groups respectively. Respective median survival times were 12.2 months (p=0.037), 12 months (p=0.050), and 7.7 months. The low dose leucovorin regimen gave a statistically significant improvement in weight gain of more than 5%, relief of symptoms, and improvement in performance status. The high dose leucovorin regimen gave a statistically significant improvement in performance status and trended toward improvement in weight gain and in relief of symptoms but these were not statistically significant.

In a second Mayo/NCCTG randomized clinical study the fluorouracil alone arm was replaced by a regimen of sequentially administered methotrexate (MTX), fluorouracil, and leucovorin. Response rates with leucovorin 200 mg/m² and fluorouracil 370 mg/m² versus leucovorin 20 mg/m² and fluorouracil 425 mg/m² versus sequential MTX and fluorouracil and leucovorin were respectively 31% (p=<0.01), 42% (p=<0.01), and 14%. Respective median survival times were 12.7 months (p=<0.04), 12.7 months (p=<0.01), and 8.4 months. No statistically significant difference in weight gain of more than 5% or in improvement in performance status was seen between the treatment arms.

In a third study comparing outcome and toxicities of low (20 mg/m²) and high-dose (200 mg/M²) leucovorin, patients received a 1-hour infusion of 400 mg/m²/day fluorouracil in addition to leucovorin every 4 weeks. The two groups were matched with no statistically significant differences in gender ratio, site of primary tumor, performance status, and tumor extent. Toxicity in the two regimens was low and not significantly different between the two groups. Overall median survival was not significantly different between the two groups: 346 days for those patients receiving low-dose leucovorin and 323 days in those patients receiving high-dose leucovorin. At 1 year, the test of equivalence was significant (p<0.01), demonstrating an absence of more than 20% benefit in 1-year survival for the high-dose regimen. The use of high-dose leucovorin combined with fluorouracil in the 5-day regimen does not significantly improve overall survival for patients who have metastatic colorectal cancer.

Finally, in a fourth large randomized study, two of the most common schedules of fluorouracil/leucovorin were compared in the treatment of advanced colorectal cancer, as each of these dosage administration schedules was demonstrated to be superior to single-agent bolus fluorouracil in previous controlled trials. Three hundred seventy-two patients with metastatic colorectal cancer were stratified according to performance status, and presence and location of any measurable indicator lesion(s) and randomized to receive chemotherapy with one of the two regimens: (1) intensive-course fluorouracil plus low-dose leucovorin (fluorouracil 425 mg/m² plus leucovorin 20 mg/m² intravenous [IV] push daily for 5 days with courses repeated at 4- to 5-week intervals); or (2) weekly fluorouracil plus high-dose leucovorin (fluorouracil 600 mg/M² IV push plus leucovorin 500 mg/m² as a 2-hour infusion weekly for 6 weeks with courses repeated every 8 weeks). There were no significant differences in therapeutic efficacy between the two fluorouracil/leucovorin regimens tested with respect to the following parameters: objective tumor response (35% v 31%), survival (median, 9.3 v 10.7 months), and palliative effects (as assessed by relief of symptoms, improved performance status, and weight gain). There were significant (P<0.05) differences in toxicity, with more leukopenia and stomatitis seen with the intensive-course regimen (Day 1-5), and more diarrhea and increased requirement for hospitalization to manage toxicity with the weekly regimen. Intensive-course fluorouracil plus low-dose leucovorin appeared to have a superior therapeutic index compared with weekly fluorouracil plus high-dose leucovorin using the dosage administration schedules applied in this study based on similar therapeutic effectiveness, but with a decreased need for hospitalization to manage chemotherapy toxicity.

3. Pharmaceutical Compositions and Uses

A compound of the present invention, a prodrug thereof or a physiologically acceptable salt of either the compound or its prodrug, can be administered as such to a human patient or it can be administered in pharmaceutical compositions in which the foregoing materials are mixed with suitable carriers or excipient(s). Techniques for formulation and administration of drugs may be found in “Remington's Pharmacological Sciences,” Mack Publishing Co., Easton, Pa., latest edition.

Routes of Administration.

General

Suitable routes of administration may include, without limitation, oral, rectal, transmucosal or intestinal administration or intramuscular, subcutaneous, intramedullary, intrathecal, direct intraventricular, intravenous, intraperitoneal, intranasal, or intraocular injections. The preferred routes of administration are oral and parenteral.

Alternatively, one may administer the compound in a local rather than systemic manner, for example, via injection of the compound directly into a solid tumor, often in a depot or sustained release formulation.

Furthermore, one may administer the drug in a targeted drug delivery system, for example, in a liposome coated with tumor-specific antibody. The liposomes will be targeted to and taken up selectively by the tumor.

Composition/Formulation

General

Pharmaceutical compositions of the present invention may be manufactured by processes well known in the art; e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the compounds of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol; cellulose preparations such as, for example, maize starch, wheat starch, rice starch and potato starch and other materials such as gelatin, gum tragacanth, methyl cellulose, hydroxypropylmethylcellulose, sodium carboxymethylcellulose, and/or polyvinylpyrrolidone (PVP). If desired, disintegrating agents may be added, such as cross-linked polyvinylpyrrolidone, agar, or alginic acid. A salt such as sodium alginate may also be used.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, or liquid polyethylene glycols. Stabilizers may be added in these formulations, also.

For administration by inhalation, the compounds for use according to the present invention are conveniently delivered in the form of an aerosol spray using a pressurized pack or a nebulizer and a suitable propellant, e.g., without limitation, dichlorodifluoromethane, trichlorofluoromethane, dichlorotetrafluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

The compounds may also be formulated for parenteral administration, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

Pharmaceutical compositions for parenteral administration include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water, before use.

The compounds may also be formulated in rectal compositions such as suppositories or retention enemas, using, e.g., conventional suppository bases such as cocoa butter or other glycerides.

In addition to the fomulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharamcologically acceptable oil), with ion exchange resins, or as a sparingly soluble derivative such as, without limitation, a sparingly soluble salt.

A non-limiting example of a pharmaceutical carrier for the hydrophobic compounds of the invention is a cosolvent system comprising benzyl alcohol, a nonpolar surfactant, a water-miscible organic polymer and an aqueous phase such as the VPD co-solvent system. VPD is a solution of 3% w/v benzyl alcohol, 8% w/v of the nonpolar surfactant Polysorbate ₈₀™, and 65% w/v polyethylene glycol 300, made up to volume in absolute ethanol. The VPD co-solvent system (VPD:D5W) consists of VPD diluted 1:1 with a 5% dextrose in water solution. This co-solvent system dissolves hydrophobic compounds well, and itself produces low toxicity upon systemic administration. The proportions of such a co-solvent system may be varied considerably without destroying its solubility and toxicity characteristics. Furthermore, the identity of the co-solvent components may be varied: for example, other low-toxicity nonpolar surfactants may be used instead of Polysorbate ₈₀™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. In addition, certain organic solvents such as dimethylsulfoxide also may be employed, although often at the cost of greater toxicity.

Additionally, the compounds may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions herein also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

3-[(2,4-dimethylpyrrol-5-yl)methylidenyl]-2-indolinone Composition.

This compound may be formulated as any of the compositions and formulations described above. A presently preferred formulation, however, is comprised of 3-[(2,4-dimethylpyrrol-5-yl)methylidenyl]-2-indolinone in sufficient sterile parenteral solution to afford a 4.5 mg/ml final concentration. Additional components of the formulation include polyethylene glycol 400; polyoxyl 35 castor oil (Cremophor®); benzyl alcohol and dehydrated alcohol. It should be noted that this formulation, since it contains Cremophor®, is not compatible with standard PVC-lined syringes, intravenous bags and administration sets.

Fluorouracil/Leucovorin Composition

Fluorouracil is commercially available in compositions and formulations which are known to those skilled in the chemotherapeutic art and may be administered in the methods of this invention as those compositions/formulations. Examples of such compositions/formulations are shown in the Package Insert which accompanies commercial fluorouracil and which is incorporated by reference as if fully set forth herein. The use of any other or different composition/formulation as such may be developed or become available in the future is also within the scope of this invention.

Likewise, leucovorin is also commercially available in compositions/formulations known to those in the chemotherapeutic art and may also be administered in the methods of this invention as those compositions/formulations. Examples of such compositions/formulations are shown in the Package Insert that accompanies commercial leucovorin and which is incorporated as if fully set forth herein. As above, any other or different composition/formulation as such may be developed or become available in the future is also within the scope of this invention.

Dosage

General

Pharmaceutical compositions suitable for use in the present invention include compositions wherein the active ingredients are contained in an amount sufficient to achieve the intended purpose; i.e., the modulation of PK activity or the treatment or prevention of a PK-related disorder.

More specifically, a therapeutically effective amount means an amount of compound effective to prevent, alleviate or ameliorate symptoms of disease or prolong the survival of the subject being treated.

Determination of a therapeutically effective amount is well within the capability of those skilled in the art, especially in light of the detailed disclosure provided herein.

For any compound used in the methods of the invention, the therapeutically effective amount or dose can be estimated initially from cell culture assays. Then, the dosage can be formulated for use in animal models so as to achieve a circulating concentration range that includes the IC₅₀ as determined in cell culture (i.e., the concentration of the test compound which achieves a half-maximal inhibition of the PK activity). Such information can then be used to more accurately determine useful doses in humans.

Toxicity and therapeutic efficacy of the compounds described herein can be determined by standard pharmaceutical procedures in cell cultures or experimental animals, e.g., by determining the IC₅₀ and the LD₅₀ (both of which are discussed elsewhere herein) for a subject compound. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide plasma levels of the active species, which are sufficient to maintain the kinase modulating effects. These plasma levels are referred to as minimal effective concentrations (MECs). The MEC will vary for each compound but can be estimated from in vitro data; e.g., the concentration necessary to achieve 50-90% inhibition of a kinase may be ascertained using the assays described herein. Dosages necessary to achieve the MEC will depend on individual characteristics and route of administration. HPLC assays or bioassays can be used to determine plasma concentrations.

Dosage intervals can also be determined using MEC value. Compounds should be administered using a regimen that maintains plasma levels above the MEC for 10-90% of the time, preferably between 30-90% and most preferably between 50-90%.

In cases of local administration or selective uptake, the effective local concentration of the drug may not be related to plasma concentration and other procedures known in the art may be employed to determine the correct dosage amount and interval.

The amount of a composition administered will, of course, be dependent on the subject being treated, the severity of the affliction, the manner of administration, the judgment of the prescribing physician, etc.

3-[(2,4-dimethylpyrrol-5-yl)methylidenyl]-2-indolinone Dosage.

Based on the pharmacological data obtained regarding 3-[(2,4-dimethylpyrrol-5-yl)methylidenyl]-2-indolinone(see above), the compound may be administered in doses ranging from about 4 mg/m² to about 195 mg/m². In a presently preferred embodiment, the dosage is between about 72.5 mg/m² and about 145 mg/m².

The dilution described in the above composition section may be administered to a patient at a rate of from about 50 to about 350 cc/hour. Preferable, the rate is from about 150 to about 250 cc/hour. Most preferably, it is from about 175 to about 225 cc/hour.

By “about,” wherever the term appears herein, is meant +10%; i.e., about 175 cc/hour means from 157.5 cc/hour to 192.5 cc/hour, etc.

In a presently preferred embodiment, the 3-[(2,4-dimethylpyrrol-5-yl)methylidenyl]-2-indolinone dose is administered during rest periods when no fluorouracil or fluorouracil/leucovorin is being administered to a patient. As was made evident by the examples in the Pharmacology section, above, fluorouracil or fluorouracil/leucovorin may be administered in numerous treatment regimes, the choice of which is within the knowledge and expertise of the treating physician.

Fluorouracil and Fluorouracil/Leucovorin Dosage

As can be seen in the clinical studies with fluorouracil and fluorouracil/leucovorin described above, there are currently a variety of schedules used for administration of fluorouracil or fluorouracil/leucovorin in advanced colorectal cancer. However, there is a remarkable lack of difference in the outcome using various administration doses and schedules of fluorouracil and fluorouracil/leucovorin, with most regimens producing leukopenia, diarrhea and mucositis to a varying degree. Thus, while fluoruracil may be administered in doses ranging from about 300 mg/m² to about 800 mg/m², schedules of fluorouracil which provide a dose intensity of approximately 400-500 mg/m²/week are presently considered to be optimal therapy. When leucovorin is included in the treatment, differences in clinical outcome for low and high dose leucovorin are minimal which, given the additional toxicity of the high dose regimen, the low dose regimen presently appears most appropriate.

Thus, while the fluorouracil or fluorouracil/leucovorin may, within the scope of this invention, be administered in any presently approved manner or in any manner found in the future to be efficacious, given the above data, a presently preferred embodiment of this invention is to administer fluorouracil at a dose of about 400 to 500 mg/m² as a bolus intravenous injection on day 1-5 of a 4 week cycle. The 4-week cycle may be repeated as necessary or until adverse side effects as recognized by the physician conducting the treatment are encountered.

Leucovorin may be administered with the fluorouracil. Leucovorin may be administered in doses of from about 20 to about 500 mg/m², preferably from about 20 to about 200 mg/m² and in a presently preferred embodiment of this invention as a low-dose administration of about 20 mg/m², also as a bolus injection, with each administration of fluorouracil.

Fluorouracil or Fluorouracil/Leucovorin in Combination with 3-[(2,4-dimethylpyrrol-5-yl)methylidenyl]-2-indolinone

It is an aspect of this invention that, when 3-[(2,4-dimethylpyrrol-5-yl)methylidenyl]-2-indolinone is administered in combination with fluorouracil or fluorouracil/leucovorin, the compounds may be administered simultaneously, sequentially, continuously, intermittently, etc. in accordance with a treatment regime calculated to take maximum advantage of the characteristics of each of the components. In a presently prefred embodiment 3-[(2,4-dimethylpyrrol-5-yl)methylidenyl]-2-indolinone is administered on days when no fluorouracil or fluorouracil/leucovorin is administered. Thus, in one embodiment of this invention, the above-described dose of 3-[(2,4-dimethylpyrrol-5-yl)methylidenyl]-2-indolinone is administered in any pattern desired; e.g., without limitation, on each day, every other day, every third day, etc. of a treatment regime selected for fluorouracil or fluorouracil/leucovorin on which fluorouracil or fluorouracil/leucovorin is not administered. The 3-[(2,4-dimethylpyrrol-5-yl)methylidenyl]-2-indolinone may be administered as a bolus intravenous injection or as a continuous intravenous infusion. However, based upon in vitro data, 3-[(2,4-dimethylpyrrol-5yl)methylidenyl]-2-indolinone may be administered over a relatively short time period (5 to 30 minutes) and exert antiproliferative activity on the endothelial cells for 3 to 4 days thereafter. Likewise, the in vivo data demonstrate that dosing with 3-[(2,4-dimethylpyrrol-5-yl)methylidenyl]-2-indolinone at 3 to 4 day intervals was sufficient to inhibit tumor growth without toxicity. Furthermore, no cumulative toxicity was observed in Phase I dose escalation studies in patients treated with up to 52 weeks of treatment. Thus, in a presently preferred embodiment of this invention, the indicated dose of 3-[(2,4-dimethylpyrrol-5-yl)methylidenyl]-2-indolinone is administered twice weekly in weeks 2-4 of each four week treatment regime.

Based on the disclosures herein, 3-[(2,4-Dimethylpyrrol-5-yl) methylene]-2-indolinone might be expected to work in combination with other chemotherapeutic agents as well. For instance, the combination of 3-[(2,4-Dimethylpyrrol-5-yl) methylene]-2-indolinone with other alkylating agents might afford synergistic activity without concomitant increased toxicity. Such alkylating agents could include, without limitation, the alkyl sulfonates; e.g., busulfan (used for treatment of chronic granulocytic leukemia), improsulfan and piposulfan; the aziridines; e.g., benzodepa, carboquone, meturedepa, and uredepa; the ethyleneimines and methylmelamines; e.g., altretamine, triethylenemelamine, triethylenephosphoramide, triethylenethiophosphoramide and trimethylolmelamine and the nitrogen mustards; e.g., chlorambucil (used in treatment of chronic lymphocytic leukemia, primary macroglobulinemia and non-Hodgkin's lymphoma), cyclophosphamide (used in treatment of Hodgkin's disease, multiple myeloma, neuroblastoma, breast cancer, ovarian cancer, lung cancer, Wilm's tumor and rhabdomyosarcoma), estramustine, ifosfamide, novembrichin, prednimustine and uracil mustard (for primary thrombocytosis, non-Hodgkin's lymphoma, Hodgkin's disease and ovarian cancer); and the triazines; e.g., dacarbazine (used for soft-tissue sarcoma).

Likewise, 3-[(2,4-Dimethylpyrrol-5-yl) methylene]-2-indolinone could have a beneficial effect in combination with other antimetabolite chemotherapeutic agents such as, without limitation, folic acid analogs (e.g., methotrexate (used in treating acute lymphocytic leukemia, choriocarcinoma, mycosis fungoides, breast, neck and head and lung cancer, osteogenic sarcoma) and pteropterin) the purine analogs such as mercaptopurine and thioguanine which find use in the treatment of acute granulocytic, acute lymphocytic and chronic granulocytic leukemias).

3-[(2,4-Dimethylpyrrol-5-yl) methylene]-2-indolinone could also prove effective in combination with natural product chemotherapeutic agents such as, without limitation, the vinca alkaloids (vinblastine (used for breast and testicular cancer), vincristine, vindesine), the epipodophylotoxins (etoposide, teniposide (both used in the treatment of testicular cancer and Kaposi's sarcoma)), the antibiotic chemotherapeutic agents (daunorubicin, doxorubicin, bleomycin, mitomycin (used for stomach, cervix, colon, breast, bladder and pancreatic cancer), dactinomycin, plicamycin, bleomycin (used for skin, esophagus and genitourinary tract cancer) and the enzymatic chemotherapeutic agents such as L-Asparaginase.

Based on the disclosures of this invention, 3-[(2,4-Dimethylpyrrol-5-yl) methylene]-2-indolinone might also benefit the activity of chemotherapeutic agents such as platinum coordination complexes (cisplatin, etc.), substituted ureas (hyroxyurea), methylhydrazine derivatives (procarbazine), adrenocortical suppressants (mitotane, aminoglutethimide) as well as hormones and antagonists such as adrenocorticosteroids (prednisone), progestins (hydroxyprogesterone caproate), estrogens (diethylstilbestrol), antiestrogens (tamoxifen) and androgens (testosterone propionate).

Finally, the combination of 3-[(2,4-Dimethylpyrrol-5-yl) methylene]-2-indolinone with mitoxantrone or paclitaxel might be expected to display especially beneficial results in the treatment of solid tumors or leukemias such as, without limitation, acute myelogenous (nonlymphocytic) leukemia.

It is to be understood that, while the above description relates to the use of 3-[(2,4-dimethylpyrrol-5-yl)methylene]-2-indolinone in combination with fluorouracil or fluorouracil/leucovorin, other compounds of this invention, in particular 3-[4-(2-carboxyethyl-3,5-dimethylpyrrol-2-yl)methylidenyl]-2-indolinone, in combination with fluorouracil or fluorouracil/leucovorin are also within the scope and spirit of this invention.

Packaging

The compositions may, if desired, be presented in a pack or dispenser device, such as an FDA approved kit, which may contain one or more unit dosage forms containing the active ingredient. The pack may for example comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or of human or veterinary administration. Such notice, for example, may be of the labeling approved by the U.S. Food and Drug Administration for prescription drugs or of an approved product insert. Compositions comprising a compound of the invention formulated in a compatible pharmaceutical carrier may also be prepared, placed in an appropriate container, and labeled for treatment of an indicated condition. Suitable conditions indicated on the label may include treatment of a tumor, inhibition of angiogenesis, treatment of fibrosis, diabetes, and the like.

4. Synthesis

The compounds of this invention, as well as the precursor 2-oxindoles and aldehydes, may be readily synthesized using techniques well known in the chemical arts. It will be appreciated by those skilled in the art that other synthetic pathways for forming the compounds of the invention are available and that the following is offered by way of example and not limitation.

1. 4-Methyl-5-(2-oxo-1,2-dihydroindol-3-ylidenemethyl)-1H-pyrrole-2-carboxylic Acid Methyl Ester

Phosphorus oxychloride (0.186 mL, 1.44 mmol) was added dropwise to a solution of dimethylormamide (0.15 mL, 1.44 mmol) in dichloromethane (4 mL) at 0° C. The mixture was warmed to room temperature and stirred for 30 minutes and then cooled to 0° C. 4-Methyl-2-pyrrolecarboxylate methyl ester (100 mg, 0.72 mmol) was added portion-wise and the mixture was then stirred at 40-50° C. for 4 hours. Sodium hydroxide (10% aqueous solution, 2 ml) was added and the reaction mixture was stirred for 30 minutes. The basic solution was then extracted with ethyl acetate (3×) and the organic layer was washed with brine to pH 6-7, dried over anhydrous sodium sulfate and concentrated under vacuum to give 115.9 mg (96%) of 4-methyl-5-formyl-2-pyrrolecarboxylate methyl ester as a yellow oil.

A mixture of oxindole (105 mg, 0.79 mmol), 4-methyl-5-formyl-2-pyrrolecarboxylate methyl ester (110 mg, 0.67 mmol) and piperidine (2 drops) in ethanol (2 mL) was stirred at 90° C. for 3 hours. The precipitate was collected by vacuum filtration, washed with ethanol and dried under vacuum to yield 153.2 mg (81%) of 4-methyl-5-(2-oxo-1,2-dihydroindol-3-ylidenomethyl) -1H-pyrrole-2-carboxylic acid methyl ester.

¹HNMR (360 MHz, DMSO-d6): 13.98 (s, br, 1H, NH), 10.97 (s, br, 1H, NH), 7.82, (d, J=7.6 Hz, 1H), 7.67 (s, 1H, H-vinyl), 7.2 (dt, J=1.2, 7.7 HZ, 1H), 7.01 (dt, J=1.2, 7.7 Hz, 1H), 6.90 (d, J=7.6 Hz, 1H), 6.77 (d, J=2 Hz, 1H).

MS (ES) 283 [M+1] (100%).

2. 4-Methyl-5-(2-oxo-1,2-dihydroindol-3-ylidenemethyl) -1H-pyrrole-2-carboxylic Acid

Phosphorus oxychloride (0.66 mL, 7.2 mmol) was added dropwise to an ice-cold solution of dimethylforamide (0.6 mL, 7.2 mmol) in dichloromethane (30 mL). The mixture was stirred at room temperature for 30 minutes and then cooled in an ice-bath. 4-methyl-2-pyrrolecarboxylate ethyl ester (919 mg, 6 mmol) was added slowly to the reaction mixture. The resulting reaction mixture was then stirred at room temperature for 2.4 hours. The mixture was then cooled in an ice-bath and 2N sodium hydroxide was added and the mixture stirred for 30 minutes. The aqueous mixture was extracted with ethyl acetate (2×), the organic layers combined and washed with brine and then dried over anhydrous sodium sulfate and concentrated under vacuum. The pink solid which was obtained was dried under vacuum at room temperature for 3 days to yield 1.05 g (96%) of 4-methyl-5-formyl-2-pyrrolecarboxylate ethyl ester. The product was used without further purification.

MS (APCI) [M−1]⁺ 180 (80%), [M−34]⁺ 146 (100%).

A mixture of 4-methyl-5-formyl-2-pyrrolecarboxylate ethyl ester (543.57 mg, 3 mmol) in 2N sodium hydroxide (1.2 g in 15 mL of water) was refluxed for ½ hour. The reaction mixture was cooled to room temperature and poured into ice water. It was then acidified to pH ˜3.5 with 2N hydrochloric acid and extracted with ethyl acetate (2×). The organic layer was washed with brine, dried over anhydrous sodium sulfate and concentrated under vacuum. The solid obtained was dried under vacuum at 40° C. for 2 hours to yield 410 mg (89%) of 4-methyl -5-formyl-2-pyrrolecarboxylic acid as a white solid.

A mixture of oxindole (133.15 mg, 1 mmol), 4-methyl-5-formyl-2-pyrrolecarboxylic acid (153.14 mg, 1 mmol), piperidine (2 drops) in ethanol (2 mL) was refluxed for 3 hours. The precipitate was collected by vacuum filtration, washed with ethanol, neutralized with 2N hydrochloric acid, washed with water and dried to yield 268.5 mg (100%) of 4-methyl-5-(2-oxo-1,2-dihydroindol-3-ylidenemethyl)-1H-pyrrole-2-carboxylic acid as an orange/red solid.

¹NMR (360 MHz, DMSO-d6): 13.84 (s, br, 1H, NH), 12.84 (s, 1-5 br, 1H, COOH), 10.98 (s, br, 1H, NH), 7.82 (d, J=7.5 Hz, 1H), 7.67 (s, 1H, H-vinyl), 7.18 (t, J=7.5 Hz, 1H), 7.01 (t, J=7.5 Hz, 1H), 6.88 (d, J=7.5 Hz, 1H), 6.71 (d, J=2.2 Hz, 1H), 2.32 (s, 3H, CH₃).

MS (negative mode) 266.8 [M−1]⁺.

3. 3-(5-Hydroxymethyl-3-melthyl-1H-pyrrol-2-ylmethylene)-1,3-dihydroindol-2-one and

4. 4-Methyl-5-(2-oxo-1,2-dihydro-indol-3-ylidenomethyl) -1H-pyrrol-2-carboxaldehyde

To a suspension of 4-methyl-5-(2-oxo-1,2-dihydroindol-3-ylidenemethyl)-1H-pyrrole-2-carboxylic acid 4.02 g, 15 mmol) in tetrahydrofuran (50 mL) was slowly added oxalyl chloride (3.80 g, 30 mmol) at 0° C. After addition was complete, the resultant suspension was stirred at room temperature for 2 hours. Sodium borohydride (1.14 g, 30 mmol) was then added portionwise to the mixture and the suspension was further stirred at room temperature for 1 day. At that thme, a second portion of 1.14 g of sodium borohydride was added followed by 10 mL of dimethylformamide to dissolve the solids and the reaction mixture was stirred for another day at room temperature. Ice water was added to the ice-cold reaction mixture until no more gas evolved. The aqueous layer was extracted with ethyl acetate. The precipitate which formed between the organic and aqueous layer was filtered, washed with water and ethyl acetate and dried to give 2.5 g (60%) of a red solid. The organic layer was washed with brine, dried over anhydrous sodium sulfate, concentrated and purified on a silica gel column eluting with ethyl acetate-hexane to give 340 mg (9%) of 3-(5-hydroxymethyl-3-methyl-1H-pyrrol-2-ylmethylone)-1,3-dihydroindol-2-one as a yellow solid and 540 mg (14% of 4-methyl-5-(2-oxo-1,2-dihydroindol-3-ylidenemethyl)-1H-pyrrole -2-carbaldehydeas an orange solid.

3-(5-Hydroxymethyl-3-methyl-1H-pyrrol-2-ylmethylene)-1,3-dihydroindol-2-one: ¹HNMR (360 MHz, DMSO-d6) 13.39 (s, br, H, NH), 10-69 (s, br, 1H, NH), 7.70(d, J=7.6 Hz, 1H), 7.56 (s, 1H, H-vinyl), 7.09 (t, J=7.6 Hz, 1H), 6.96 (t, J=7.6 Hz, 1H), 6.86 (d, J=7.6 Hz, 1H), 6.06 (d, J=2.1 Hz, 1H), 5.33 (t, J=5, 6 Hz, 1H, OH), 4.51 (d, J=5, 6 Hz, 2H, CH₂OH), 2.31 (s, 3H, CH₃)

MS 251 [M−1]⁺ (100%)

M.p. >350° C.

4-Methyl-5-(2-oxo-1,2-dihydroindol-3-ylidenemethyl)-1H-pyrrole -2-carbaldehyde: ¹HNMR (360 MHz, DMSO-d6) δ: 13.87 (s, br, 1H, NH), 11.05 (s, br, 1H, NH), 9.61(s, 1H CHO), 7.85 (d, J=7.5 Hz, 1H), 7.71 (s, 1H, H-vinyl), 7.23 (t, J=7.5 Hz, 1H), 7.03, (t, J=7.5 Hz, 1H), 6.97 (d, J=2.2 Hz, 1H), 6.9 (d, J=7.5 Hz, 1H), 2.36 (s, 3H, CH₃).

MS 237.4 [M−OH]⁺ 9100%).

M.p. 267.3-268.4° C.

5. Biological Evaluation

It will be appreciated that, in any given series of compounds, a spectrum of biological activities will be obtained. In its preferred embodiments, this invention relates to novel 3-heteroarylidenyl-2-indolinones demonstrating the ability to modulate RTK, CTK, and STK activity. The following assays are employed to select those compounds demonstrating the optimal degree of the desired activity.

Assay Procedures.

The following in vitro assays may be used to determine the level of activity and effect of the different compounds of the present invention on one or more of the PKs. Similar assays can be designed along the same lines for any PK using techniques Well known in the art.

The cellular/catalytic assays described herein are performed in an ELISA format. The general procedure is as follows: a compound is introduced to cells expressing the test kinase, either naturally or recombinantly, for a selected period of time after which, if the test kinase is a receptor, a ligand known to activate the receptor is added. The cells are lysed and the lysate is transferred to the wells of an ELISA plate previously coated with a specific antibody recognizing the substrate of the enzymatic phosphorylation reaction. Non-substrate components of the cell lysate are washed away and the amount of phosphorylation on the substrate is detected with an antibody specifically recognizing phosphotyrosine compared with control cells that were not contacted with a test compound. The cellular/biologic assays described herein measure the amount of DNA made in response to activation of a test kinase, which is a general measure of a proliferative response. The general procedure for this assay is as follows: a compound is introduced to cells expressing the test kinase, either naturally or recombinantly, for a selected period of time after which, if the test kinase is a receptor, a ligand known to activate the receptor is added. After incubation at least overnight, a DNA labeling reagent such as Bromodeoxyuridine (BrdU) or 3H-thymidine is added. The amount of labeled DNA is detected with either an anti-BrdU antibody or by measuring radioactivity and is compared to control cells not contacted with a test compound.

Cellular/Catalytic Assays

Enzyme linked immunosorbent assays (ELISA) may be used to detect and measure the presence of PK activity. The ELISA may be conducted according to known protocols which are described in, for example, Voller, et al., 1980, “Enzyme-Linked Immunosorbent Assay,” In: Manual of Clinical Immunology, 2d ed., edited by Rose and Friedman, pp 359-371 Am. Soc. Of Microbiology, Washington, D.C.

The disclosed protocol may be adapted for determining activity with respect to a specific PK. That is, the preferred protocols for conducting the ELISA experiments for specific PKs is provided below. However, adaptation of these protocols for determining a compound's activity for other members of the RTK family, as well as for CTKs and STKs, is well within the scope of knowledge of those skilled in the art.

FLK-1 Assay

An ELISA assay is conducted to measure the kinase activity of the FLK-1 receptor and more specifically, the inhibition or activation of TK activity on the FLK-1 receptor. Specifically, the following assay can be conducted to measure kinase activity of the FLK-1 receptor in cells genetically engineered to express Flk-1.

Materials and Reagents.

a. Corning 96-well ELISA plates (Corning Catalog No. 25805-96);

b. Cappel goat anti-rabbit IgG (catalog no. 55641);

c. PBS (Gibco Catalog No. 450-1300EB);

d. TBSW Buffer (50 mM Tris (pH 7.2), 150 mM NaCl and 0.1% Tween-20);

e. Ethanolamine stock (10% ethanolamine (pH 7.0), stored at 4° C.);

f. HNTG buffer (20 mM HEPES buffer (pH 7.5), 150 mM NaCl, 0.2% Triton X-100, and 10% glycerol);

g. EDTA (0.5 M (pH 7.0) as a 100×stock);

h. Sodium orthovanadate (0.5 M as a 100×stock);

i. Sodium pyrophosphate (0.2 M as a 100×stock);

j. NUNC 96 well V bottom polypropylene plates (Applied Scientific Catalog No. AS-72092);

k. NIH3T3 C7#3 Cells (FLK-1 expressing cells);

l. DMEM with 1×high glucose L-Glutamine (catalog No. 11965-050);

m. FBS, Gibco (catalog no. 16000-028);

n. L-glutamine, Gibco (catalog no. 25030-016);

o. VEGF, PeproTech, Inc. (catalog no. 100-20)(kept as 1 μg/100 μl stock in Milli-Q dH₂O and stored at −20° C.;

p. Affinity purified anti-FLK-1 antiserum;

q. UB40 monoclonal antibody specific for phosphotyrosine (see, Fendley, et al., 1990, Cancer Research 50:1550-1558);

r. EIA grade Goat anti-mouse IgG-POD (BioRad catalog no. 172-1011);

s. 2,2-azino-bis(3-ethylbenz-thiazoline-6-sulfonic acid (ABTS) solution (100 mM citric acid (anhydrous), 250 mM Na₂HPO₄ (pH 4.0), 0.5 mg/ml ABTS (Sigma catalog no. A-1888)), solution should be stored in dark at 4° C. until ready for use;

t. H₂O₂ (30% solution) (Fisher catalog no. H325);

u. ABTS/H₂O₂ (15 ml ABTS solution, 2 μl H₂O₂) prepared 5 minutes before use and left at room temperature;

v. 0.2 M HCl stock in H₂O;

w. dimethylsulfoxide (100%)(Sigma Catalog No. D-8418); and

y. Trypsin-EDTA (Gibco BRL Catalog No. 25200-049).

Protocol.

1. Coat Corning 96-well ELISA plates with 1.0 μg per well Cappel Anti-rabbit IgG antibody in 0.1M Na₂CO₃ pH 9.6. Bring final volume to 150 μl per well. Store plates overnight at 4° C. Plates can be kept up to two weeks when stored at 4° C.

2. Grow cells in Growth media (DMEM, supplemented with 2.0 mM L-Glutamine, 10% FBS) in suitable culture dishes until confluent at 37° C., 5% CO₂.

3. Harvest cells by trypsinization and seed in Corning 25850 polystyrene 96-well round bottom cell plates, 25,000 cells/well in 200 μl of growth media.

4. Grow cells at least one day at 37° C., 5% CO₂.

5. Wash cells with D-PBS 1×.

6. Add 200 μl/well of starvation media (DMEM, 2.0 mM 1-Glutamine, 0.1% FBS). Incubate overnight at 37° C., 5% CO₂.

7. Dilute Compounds 1:20 in polypropylene 96 well plates using starvation media. Dilute dimethylsulfoxide 1:20 for use in control wells.

8. Remove starvation media from 96 well cell culture plates and add 162 μl of fresh starvation media to each well.

9. Add 18 μl of 1:20 diluted compound dilution (from step 7) to each well plus the 1:20 dimethylsulfoxide dilution to the control wells (+/−VEGF), for a final dilution of 1:200 after cell stimulation. Final dimethylsulfoxide is 0.5%. Incubate the plate at 37° C., 5% CO₂ for two hours.

10. Remove unbound antibody from ELISA plates by inverting plate to remove liquid. Wash 3 times with TBSW+0.5% ethanolamine, pH 7.0. Pat the plate on a paper towel to remove excess liquid and bubbles.

11. Block plates with TBSW+0.5% ethanolamine, pH 7.0, 150 μl per well. Incubate plate thirty minutes while shaking on a microtiter plate shaker.

12. Wash plate 3 times as described in step 10.

13. Add 0.5 μg/well affinity purified anti-FLU-1 polyclonal rabbit antiserum. Bring final volume to 150 μl/well with TBSW+0.5% ethanolamine pH 7.0. Incubate plate for thirty minutes while shaking.

14. Add 180 μl starvation medium to the cells and stimulate cells with 20 μl/well 10.0 mM sodium orthovanadate and 500 ng/ml VEGF (resulting in a final concentration of 1.0 mM sodium orthovanadate and 50 ng/ml VEGF per well) for eight minutes at 37° C., 5% CO₂. Negative control wells receive only starvation medium.

15. After eight minutes, media should be removed from the cells and washed one time with 200 μl/well PBS.

16. Lyse cells in 150 μl/well HNTG while shaking at room temperature for five minutes. HNTG formulation includes sodium ortho vanadate, sodium pyrophosphate and EDTA.

17. Wash ELISA plate three times as described in step 10.

18. Transfer cell lysates from the cell plate to ELISA plate and incubate while shaking for two hours. To transfer cell lysate pipette up and down while scrapping the wells.

19. Wash plate three times as described in step 10.

20. Incubate ELISA plate with 0.02 μg/well UB40 in TBSW+05% ethanolamine. Bring final volume to 150 μl/well. Incubate while shaking for 30 minutes.

21. Wash plate three times as described in step 10.

22. Incubate ELISA plate with 1:10,000 diluted EIA grade goat anti-mouse IgG conjugated horseradish peroxidase in TBSW plus 0.5% ethanolamine, pH 7.0. Bring final volume to 150 μl/well. Incubate while shaking for thirty minutes.

23. Wash plate as described in step 10.

24. Add 100 μl of ABTS/H₂O₂ solution to well. Incubate ten minutes while shaking.

25. Add 100 μl of 0.2 M HCl for 0.1 M HCl final concentration to stop the color development reaction. Shake 1 minute at room temperature. Remove bubbles with slow stream of air and read the ELISA plate in an ELISA plate reader at 410 nm.

EGF Receptor-HER2 Chimeric Receptor Assay in Whole Cells.

HER2 kinase activity in whole EGFR-NIH3T3 cells are measured as described below:

Materials and Reagents.

a. EGF: stock concentration: 16.5 ILM; EGF 201, TOYOBO, Co., Ltd. Japan.

b. 05-101 (UBI) (a monoclonal antibody recognizing an EGFR extracellular domain).

c. Anti-phosphotyrosine antibody (anti-Ptyr) (polyclonal)(see, Fendley, et al., supra).

d. Detection antibody: Goat anti-rabbit IgG horseradish peroxidase conjugate, TAGO, Inc., Burlingame, Calif.

| e. | TBST buffer: | | |
|---|---|---|---|
| | Tris-HCl, pH 7.2 | 50 | mM |
| | NaCl | 150 | mM |
| | Triton X-100 | 0.1 | |
| f. | HNTG 5X stock: | | |
| | HEPES | 0.1 | M |
| | NaCl | 0.75 | M |

| | | |
|---|---|---|
| | Glycerol | 50% |
| | Triton X-100 | 1.0% |
| g. | ABTS stock: | |

| | | |
|---|---|---|
| Citric Acid | 100 | mM |
| Na₂HPO₄ | 250 | mM |
| HCl, conc. | 0.5 | mM |
| ABTS* | 0.5 | mg/ml |

* (2,2′-azinobis(3-ethylbenzthiazolinesulfonic acid)). Keep solution in dark at 4° C. until use.

h. Stock reagents of:

EDTA 100 mM pH 7.0

Na₃VO₄ 0.5 M

Na₄(P₂O₇) 0.2 M

Procedure.

Pre-Coat ELISA Plate

1. Coat ELISA plates (Corning, 96 well, Cat. #25805-96) with 05-101 antibody at 0.5 μg per well in PBS, 100 μl final volume/well, and store overnight at 4° C. Coated plates are good for up to 10 days when stored at 4° C.

2. On day of use, remove coating buffer and replace with 100 μl blocking buffer (5% Carnation Instant Non-Fat Dry Milk in PBS). Incubate the plate, shaking, at room temperature (about 23° C. to 25° C.) for 30 minutes. Just prior to use, remove blocking buffer and wash plate 4 times with TBST buffer.

Seeding Cells

1. An NIH3T3 cell line overexpressing a chimeric receptor containing the EGFR extracellular domain and intracellular HER2 kinase domain can be used for this assay.

2. Choose dishes having 80-90% confluence for the experiment. Trypsinize cells and stop reaction by adding 10% fetal bovine serum. Suspend cells in DMEM medium (10% CS DMEM medium) and centrifuge once at 1500 rpm, at room temperature for 5 minutes.

3. Resuspend cells in seeding medium (DMEM, 0.5% bovine serum), and count the cells using trypan blue. Viability above 90% is acceptable. Seed cells in DMEM medium (0.5% bovine serum) at a density of 10,000 cells per well, 100 μl per well, in a 96 well microtiter plate. Incubate seeded cells in 5% CO₂ at 37° C. for about 40 hours.

Assay Procedures

1. Check seeded cells for contamination using an inverted microscope. Dilute drug stock (10 mg/ml in DMSO) 1:10 in DMEM medium, then transfer 5 μl to a TBST well for a final drug dilution of 1:200 and a final DMSO concentration of 1%. Control wells receive DMSO alone. Incubate in 5% CO₂ at 37° C. for two hours.

2. Prepare EGF ligand: dilute stock EGF in DMEM so that upon transfer of 10 μl dilute EGF (1:12 dilution), 100 nM final concentration is attained.

3. Prepare fresh HNTG* sufficient for 100 μl per well; and place on ice.

HNTG* (10 ml):

| | |
|---|---|
| HNTG stock | 2.0 ml |
| milli-Q H₂O | 7.3 ml |
| EDTA, 100 mM, pH 7.0 | 0.5 ml |
| Na₃VO₄ (0.5 M) | 0.1 ml |
| Na₄(P₂O₇) (0.2 M) | 0.1 ml |

4. After 120 minutes incubation with drug, add prepared SGF ligand to cells, 10 μl per well, to a final concentration of 100 nM. Control wells receive DMEM alone. Incubate with shaking, at room temperature, for 5 minutes.

5. Remove drug, EGF, and DMEM. Wash cells twice with PBS. Transfer HNTG* to cells, 100 μl per well. Place on ice for 5 minutes. Meanwhile, remove blocking buffer from other ELISA plate and wash with TBST as described above.

6. With a pipette tip securely fitted to a micropipettor, scrape cells from plate and homogenize cell material by repeatedly aspirating and dispensing the HNTG* lysis buffer. Transfer lysate to a coated, blocked, and washed ELISA plate. Incubate shaking at room temperature for one hour.

7. Remove lysate and wash 4 times with TBST. Transfer freshly diluted anti-Ptyr antibody to ELISA plate at 100 μl per well. Incubate shaking at room temperature for 30 minutes in the presence of the anti-Ptyr antiserum (1:3000 dilution in TBST).

8. Remove the anti-Ptyr antibody and wash 4 times with TBST. Transfer the freshly diluted TAGO anti-rabbit IgG antibody to the ELISA plate at 100 μl per well. Incubate shaking at room temperature for 30 minutes (anti-rabbit IgG antibody: 1:3000 dilution in TBST).

9. Remove TAGO detection antibody and wash 4 times with TBST. Transfer freshly prepared ABTS/H₂O₂ solution to ELISA plate, 100 μl per well. Incubate shaking at room temperature for 20 minutes. (ABTS/H₂O₂ solution: 1.0 μl 30% H₂O₂ in 10 ml ABTS stock).

10. Stop reaction by adding 50 μl 5N H₂SO₄ (optional), and determine O.D. at 410 nm.

11. The maximal phosphotyrosine signal is determined by subtracting the value of the negative controls from the positive controls. The percent inhibition of phosphotyrosine content for extract-containing wells is then calculated, after subtraction of the negative controls.

PDGF-R Assay

All cell culture media, glutamine, and fetal bovine serum can be purchased from Gibco Life Technologies (Grand Island, N.Y.) unless otherwise specified. All cells are grown in a humid atmosphere of 90-95% air and 5-10% CO₂ at 37° C. All cell lines are routinely subcultured twice a week and are negative for mycoplasma as determined by the Mycotect method (Gibco).

For ELISA assays, cells (U1242, obtained from Joseph Schlessinger, NYU) are grown to 80-90% confluency in growth medium (MEM with 10% FBS, NEAA, 1 mM NaPyr and 2 mM GLN) and seeded in 96-well tissue culture plates in 0.5% serum at 25,000 to 30,000 cells per well. After overnight incubation in 0.5% serum-containing medium, cells are changed to serum-free medium and treated with test compound for 2 hr in a 5% CO₂, 37° C. incubator. Cells are then stimulated with ligand for 5-10 minute followed by lysis with HNTG (20 mM Hepes, 150 mM NaCl, 10% glycerol, 5 mM EDTA, 5 mM Na₃VO₄, 0.2% Triton X-100, and 2 mM NaPyr). Cell lysates (0.5 mg/well in PBS) are transferred to ELISA plates previously coated with receptor-specific antibody and which had been blocked with 5% milk in TBST (50 mM Tris-HCl pH 7.2, 150 mM NaCl and 0.1% Triton X-100) at room temperature for 30 min. Lysates are incubated with shaking for 1 hour at room temperature. The plates are washed with TBST four times and then incubated with polyclonal anti-phosphotyrosine antibody at room temperature for 30 minutes. Excess anti-phosphotyrosine antibody is removed by rinsing the plate with TBST four times. Goat anti-rabbit IgG antibody is added to the ELISA plate for 30 min at room temperature followed by rinsing with TBST four more times. ABTS (100 mM citric acid, 250 mM Na₂HPO₄ and 0.5 mg/mL 2,2-azino-bis(3-ethylbenzthiazoline-6-sulfonic acid)) plus H₂O₂ (1.2 mL 30% H₂O₂ to 10 ml ABTS) is added to the ELISA plates to start color development. Absorbance at 410 nm with a reference wavelength of 630 nm is recorded about 15 to 30 min after ABTS addition.

IGF-1 Receptor Assay

The following protocol may be used to measure phosphotyrosine level on IGF-1 receptor, which indicates IGF-1 receptor tyrosine kinase activity.

Materials and Reagents.

a. The cell line used in this assay is 3T3/IGF-1R, a cell line genetically engineered to overexpresses IGF-1 receptor.

b. NIH3T3/IGF-1R is grown in an incubator with 5% CO₂ at 37° C. The growth media is DMEM+10% FBS (heat inactivated)+2 mM L-glutamine.

c. Affinity purified anti-IGF-1R antibody 17-69.

d. D-PBS:

| | |
|---|---|
| KH₂PO₄ | 0.20 g/l |
| KH₂PO₄ | 2.16 g/l |
| KCl | 0.20 g/l |
| NaCl | 8.00 g/l (pH 7.2) |

e. Blocking Buffer: TBST plus 5% Milk (Carnation Instant Non-Fat Dry Milk).

f. TBST buffer:

| Tris-HCl | 50 mM |
|---|---|
| NaCl | 150 mM (pH 7.2/HCl 10 N) |
| Triton X-100 | 0.1% |

Stock solution of TBS (10×) is prepared, and Triton X-100 is added to the buffer during dilution.

g. HNTG buffer:

| HEPES | 20 mM |
|---|---|
| NaCl | 150 mM (pH 7.2/HCl 1 N) |
| Glycerol | 10% |
| Triton X-100 | 0.2% |

Stock solution (5×) is prepared and kept at 4° C.

h. EDTA/HCl: 0.5 M pH 7.0 (NaOH) as 10OX stock.

i. Na₃VO₄: 0.5 M as 100×stock and aliquots are kept at 80° C.

j. Na₄P₂O₇: 0.2 M as 100×stock.

k. Insulin-like growth factor-1 from Promega (Cat#G5111).

l. Rabbit polyclonal anti-phosphotyrosine antiserum.

m. Goat anti-rabbit IgG, POD conjugate (detection antibody), Tago (Cat. No. 4520, Lot No. 1802): Tago, Inc., Burlingame, Calif.

n. ABTS (2,2′-azinobis(3-ethylbenzthiazolinesulfonic acid)) solution:

| | | |
|---|---|---|
| Citric acid | 100 | mM |
| Na₂HPO₄ | 250 | mM (pH 4.0/1 N HCl) |
| ABTS | 0.5 | mg/ml |

ABTS solution should be kept in dark and 4° C. The solution should be discarded when it turns green.

o. Hydrogen Peroxide: 30% solution is kept in the dark and at 4° C.

Procedure.

All the following steps are conducted at room temperature unless specifically indicated otherwise. All ELISA plate washings are performed by rinsing the plate with tap water three times, followed by one TBST rinse. Pat plate dry with paper towels.

Cell Seeding:

1. The cells, grown in tissue culture dish (Corning 25020-100) to 80-90% confluence, are harvested with Trypsin-EDTA (0.25%, 0.5 ml/D-100, GIBCO).

2. Resuspend the cells in fresh DMEM+10% FBS+2 mM L-Glutamine, and transfer to 96-well tissue culture plate (Corning, 25806-96) at 20,000 cells/well (100 μl/well). Incubate for 1 day then replace medium to serum-free medium (90 μl) and incubate in 5% CO₂ and 37° C. overnight.

ELISA Plate Coating and Blocking:

1. Coat the ELISA plate (Corning 25805-96) with Anti-IGF-1R Antibody at 0.5 μg/well in 100 μl PBS at least 2 hours.

2. Remove the coating solution, and replace with 100 μl Blocking Buffer, and shake for 30 minutes. Remove the blocking buffer and wash the plate just before adding lysate.

Assay Procedures:

1. The drugs are tested under serum-free condition.

2. Dilute drug stock (in 100% DMSO) 1:10 with DMEM in 96-well poly-propylene plate, and transfer 10 μl/well of this solution to the cells to achieve final drug dilution 1:100, and final DMSO concentration of 1.0%. Incubate the cells in 5% CO₂ at 37° C. for 2 hours.

3. Prepare fresh cell lysis buffer (HNTG*)

| | |
|---|---|
| HNTG | 2 ml |
| EDTA | 0.1 ml |
| Na₃VO₄ | 0.1 ml |
| Na₄(P₂O₇₎ | 0.1 ml |
| H₂O | 7.3 ml |

4. After drug incubation for two hours, transfer 10 μl/well of 200 nM IGF-1 Ligand in PBS to the cells (Final Conc. is 20 nM), and incubate at 5% CO₂ at 37° C. for 10 minutes.

5. Remove media and add 100 μl/well HNTG* and shake for 10 minutes. Look at cells under microscope to see if they are adequately lysed.

6. Use a 12-channel pipette to scrape the cells from the plate, and homogenize the lysate by repeated aspiration and dispensing. Transfer all the lysate to the antibody coated ELISA plate, and shake for 1 hour.

7. Remove the lysate, wash the plate, transfer anti-pTyr (1:3,000 with TBST) 100 μl/well, and shake for 30 minutes.

8. Remove anti-pTyr, wash the plate, transfer TAGO (1:3,000 with TBST) 100 μl/well, and shake for 30 minutes.

9. Remove detection antibody, wash the plate, and transfer fresh ABTS/H₂O₂ (1.2 μl H₂O₂ to 10 ml ABTS) 100 μl/well to the plate to start color development.

Measure OD at 410 nm with a reference wavelength of 630 nm in Dynatec MR5000.

EGFR Assay

EGF Receptor kinase activity in cells genetically engineered to express human EGF-R can be measured as described below:

Materials and Reagents.

a. EGF Ligand: stock concentration=16.5 μM; EGF 201, TOYOBO, Co., Ltd. Japan.

b. 05-101 (UBI) (a monoclonal antibody recognizing an EGFR extracellular domain).

c. Anti-phosphotyosine antibody (anti-Ptyr) (polyclonal).

d. Detection antibody: Goat anti-rabbit lgG horse radish peroxidase conjugate, TAGO, Inc., Burlingame, Calif.

| e. | TEST buffer: | | |
|---|---|---|---|
| | Tris-HCl, pH 7 | 50 | mM |
| | NaCl | 150 | mM |
| | Triton X-100 | 0.1 | |
| f. | HNTG 5X stock: | | |
| | HEPES | 0.1 | M |
| | NaCl | 0.75 | M |
| | Glycerol | 50 | |

| | | |
|---|---|---|
| | Triton X-100 | 1.0% |
| g. | ABTS stock: | |

| | | |
|---|---|---|
| Citric Acid | 100 | mM |
| Na₃VO₄ | 250 | mM |
| HCl, conc. | 4.0 | pH |
| ABTS* | 0.5 | mg/ml |

Keep solution in dark at 4° C. until used.

| | |
|---|---|
| h. | Stock reagents of: |
| | EDTA 100 mM pH 7.0 |
| | Na₃VO₄ 0.5 M |
| | Na₄(P₂O₇) 0.2 M |

Procedure

Pre-Coat ELISA Plate

1. Coat ELISA plates (Corning, 96 well, Cat. #25805-96) with 05-101 antibody at 0.5 μg per well in PBS, 150 μl final volume/well, and store overnight at 4° C. Coated plates are good for up to 10 days when stored at 4° C.

2. On day of use, remove coating buffer and replace with blocking buffer (5% Carnation Instant NonFat Dry Milk in PBS). Incubate the plate, shaking, at room temperature (about 23° C. to 25° C.) for 30 minutes. Just prior to use, remove blocking buffer and wash plate 4 times with TBST buffer.

Seeding Cells

1. NIH 3T3/C7 cell line (Honegger, et al., Cell 51:199-209, 1987) can be use for this assay.

2. Choose dishes having 80-90% confluence for the experiment. Trypsinize cells and stop reaction by adding 10% CS DMEM medium. Suspend cells in DMEM medium (10% CS DMEM medium) and centrifuge once at 1000 rpm at room temperature for 5 minutes.

3. Resuspend cells in seeding medium (DMEM, 0.5% bovine serum), and count the cells using trypan blue. Viability above 90% is acceptable. Seed cells in DMEM medium (0.5% bovine serum) at a density of 10,000 cells per well, 100 μl per well, in a 96 well microtiter plate. Incubate seeded cells in 5% CO₂ at 37° C. for about 40 hours.

Assay Procedures

1. Check seeded cells for contamination using an inverted microscope. Dilute test compounds stock (10 mg/ml in DMSO) 1:10 in DMEM medium, then transfer 5 μl to a test well for a test compounds drug dilution of 1:200 and a final DMSO concentration of 1%. Control wells receive DMSO alone. Incubate in 5% CO₂ at 37° C. for one hour.

2. Prepare EGF ligand: dilute stock EGF in DMEM so that upon transfer of 10 μl dilute EGF (1:12 dilution), 25 nM final concentration is attained.

3. Prepare fresh 10 ml HNTG* sufficient for 100 μl per well wherein HNTG* comprises: HNTG stock (2.0 ml), milli-Q H₂O (7.3 ml), EDTA, 100 mM, pH 7.0 (0.5 ml), Na₃VO₄ 0.5 M (0.1 ml) and Na₄(P₂O₇), 0.2 M (0.1 ml).

4. Place on ice.

5. After two hours incubation with drug, add prepared EGF ligand to cells, 10 μl per well, to yield a final concentration of 25 nM. Control wells receive DMEM alone. Incubate, shaking, at room temperature, for 5 minutes.

6. Remove test compound, EGF, and DMEM. Wash cells twice with PBS. Transfer HNTG* to cells, 100 μl per well. Place on ice for 5 minutes. Meanwhile, remove blocking buffer from other ELISA plate and wash with TBST as described above.

7. With a pipette tip securely fitted to a micropipettor, scrape cells from plate and homogenize cell material by repeatedly aspirating and dispensing the HNTG* lysis buffer. Transfer lysate to a coated, blocked, and washed ELISA plate. Incubate shaking at room temperature for one hour.

8. Remove lysate and wash 4 times with TBST. Transfer freshly diluted anti-Ptyr antibody to ELISA plate at 100 μl per well. Incubate shaking at room temperature for 30 minutes in the presence of the anti-Ptyr antiserum (1:3000 dilution in TBST).

9. Remove the anti-Ptyr antibody and wash 4 times with TBST. Transfer the freshly diluted TAGO 30 anti-rabbit IgG antibody to the ELISA plate at 100 μl per well. Incubate shaking at room temperature for 30 minutes (anti-rabbit IgG antibody: 1:3000 dilution in TBST).

10. Remove detection antibody and wash 4 times with TBST. Transfer freshly prepared ABTS/H₂O₂ solution to ELISA plate, 100 μl per well. Incubate at room temperature for 20 minutes. ABTS/H₂O₂ solution: 1.2 μl 30% H₂O₂ in 10 ml ABTS stock.

11. Stop reaction by adding 50 μl 5N H₂SO₄ (optional), and determine O.D. at 410 nm.

12. The maximal phosphotyrosine signal is determined by subtracting the value of the negative controls from the positive controls. The percent inhibition of phosphotyrosine content for extract-containing wells is then calculated, after subtraction of the negative controls.

Met Autophosphorylation Assay

This assay determines Met tyrosine kinase activity by analyzing Met protein tyrosine kinase levels on the Met receptor.

Reagents

a. HNTG (5×stock solution): Dissolve 23.83 g HEPES and 43.83 g NaCl in about 350 ml dH₂O. Adjust pH to 7.2 with HCl or NaOH, add 500 ml glycerol and 10 ml Triton X-100, mix, add dH₂O to 1 L total volume. To make 1 L of 1×working solution add 200 ml 5×stock solution to 800 ml dH₂O, check and adjust pH as necessary, store at 4° C.

b. PBS (Dulbecco's Phosphate-Buffered Saline), Gibco Cat. #450-1300EB (1×solution).

c. Blocking Buffer: in 500 ml dH₂O place 100 g BSA, 12.1 g Tris-pH 7.5, 58.44 g NaCl and 10 ml Tween-20, dilute to 1 L total volume.

d. Kinase Buffer: To 500 ml dH₂O add 12.1 g TRIS (pH 7.2), 58.4 g NaCl, 40.7 g MgCl₂ and 1.9 g EGTA; bring to 1 L total volume with dH₂O.

e. PMSF (Phenylmethylsulfonyl fluoride), Sigma Cat. #P-7626, to 435.5 mg, add 100% ethanol to 25 ml total volume, vortex.

f. ATP (Bacterial Source), Sigma Cat. #A-7699, store powder at −20° C.; to make up solution for use, dissolve 3.31 mg in 1 ml dH₂O.

9. RC-20H HRPO Conjugated Anti-Phosphotyrosine, Transduction Laboratories Cat. #E120H.

h. Pierce 1-Step TM Turbo TMB-ELISA (3,3′,5,5′-tetramethylbenzidine, Pierce Cat. #34022.

i. H₂SO₄, add 1 ml conc.(18 N) to 35 ml dH₂O.

j. TRIS HCL, Fischer Cat. #BP152-5; to 121.14 g of material, add 600 ml MilliQ H₂O, adjust pH to 7.5 (or 7.2) with HCl, bring volume to 1 L with MilliQ H₂O.

k. NaCl, Fischer Cat. #S271-10, make up 5M solution.

l. Tween-20, Fischer Cat. #S337-500.

m. Na₃VO₄, Fischer Cat. #S454-50, to 1.8 g material add 80 ml MilliQ H₂O, adjust pH to 10.0 with HCl or NaOH, boil in microwave, cool, check pH, repeat procedure until pH stable at 10.0, add MilliQ H₂O to 100 ml total volume, make 1 ml aliquots and store at −80° C.

n. MgCl₂, Fischer Cat. #M33-500, make up 1M solution.

o. HEPES, Fischer Cat. #BP310-500, to 200 ml MilliQ H₂O, add 59.6 g material, adjust pH to 7.5, bring volume to 250 ml total, sterile filter.

p. Albumin, Bovine (BSA), Sigma Cat. #A-4503, to 30 grams material add sterile distilled water to make total volume of 300 ml, store at 4° C.

q. TBST Buffer: to approx. 900 ml dH₂O in a 1 L graduated cylinder add 6.057 g TRIS and 8.766 g NaCl, when dissolved, adjust pH to 7.2 with HCl, add 1.0 ml Triton X-100 and bring to 1 L total volume with dH₂O.

r. Goat Affinity purified antibody Rabbit IgG (whole molecule), Cappel Cat. #55641.

s. Anti h-Met (C-28) rabbit polyclonal IgG antibody, Santa Cruz Chemical Cat. #SC-161.

t. Transiently Transfected EGFR/Met chimeric cells (EMR) (Komada, et al., Oncogene, 8:2381-2390 (1993).

u. Sodium Carbonate Buffer, (Na₂CO₄, Fischer Cat. #S495): to 10.6 g material add 800 ml MilliQ H₂O, when dissolved adjust pH to 9.6 with NaOH, bring up to 1 L total volume with MilliQ H₂O, filter, store at 4° C.

Procedure

All of the following steps are conducted at room temperature unless it is specifically indicated otherwise. All ELISA plate washing is by rinsing 4×with TBST.

EMR Lysis

This procedure can be performed the night before or immediately prior to the start of receptor capture.

1. Quick thaw lysates in a 37° C. waterbath with a swirling motion until the last crystals disappear.

2. Lyse cell pellet with 1×HNTG containing 1 mM PMSF. Use 3 ml of HNTG per 15 cm dish of cells. Add ½ the calculated HNTG volume, vortex the tube for 1 min., add the remaining amount of HNTG, vortex for another min.

3. Balance tubes, centrifuge at 10,000×g for 10 min at 4° C.

4. Pool supernatants, remove an aliquot for protein determination.

5. Quick freeze pooled sample in dry ice/ethanol bath. This step is performed regardless of whether lysate will be stored overnight or used immediately following protein determination.

6. Perform protein determination using standard bicinchoninic acid (BCA) method (BCA Assay Reagent Kit from Pierce Chemical Cat. #23225).

ELISA Procedure

1. Coat Corning 96 well ELISA plates with 5 μg per well Goat anti-Rabbit antibody in Carbonate Buffer for a total well volume of 50 μl. Store overnight at 4° C.

2. Remove unbound Goat anti-rabbit antibody by inverting plate to remove liquid.

3. Add 150 μl of Blocking Buffer to each well. Incubate for 30 min. with shaking.

4. Wash 4×with TBST. Pat plate on a paper towel to remove excess liquid and bubbles.

5. Add 1 g per well of Rabbit anti-Met antibody diluted in TBST for a total well volume of 100 μl.

6. Dilute lysate in HNTG (90 μg lysate/1001) 7. Add 100 μl of diluted lysate to each well. Shake for 60 min.

8. Wash 4×with TBST. Pat on paper towel to remove excess liquid and bubbles.

9. Add 50 μl of 1×lysate buffer per well.

10. Dilute compounds/extracts 1:10 in 1×Kinase Buffer in a polypropylene 96 well plate.

11. Transfer 5.5 μl of diluted compound to ELISA plate wells. Incubate at room temperature with shaking for 20 min.

12. Add 5.5 μl of 60 μM ATP solution per well. Negative controls do not receive any ATP. Incubate for 90 min., with shaking.

13. Wash 4×with TBST. Pat plate on paper towel to remove excess liquid and bubbles.

14. Add 100 μl per well of RC20 (1:3000 dilution in Blocking Buffer). Incubate 30 min. with shaking.

15. Wash 4×with TBST. Pat plate on paper towel to remove excess liquid and bubbles.

16. Add 100 μl per well of Turbo-TMB. Incubate with shaking for 30-60 min.

17. Add 100 μl per well of 1M H₂SO₄ to stop reaction.

18. Read assay on Dynatech MR7000 ELISA reader. Test Filter =450 nm, reference filter =410 nm.

Biochemical src Assay

This assay is used to determine src protein kinase activity measuring phosphorylation of a biotinylated peptide as the readout.

Materials and Reagents:

a. Yeast transformed with src (Sugen, Inc., Redwood City, Calif.).

b. Cell lysates: Yeast cells expressing src are pelleted, washed once with water, re-pelleted and stored at −80° C. until use.

c. N-terminus biotinylated EEEYEEYEEEYEEEYEEEY is prepared by standard procedures well known to those skilled in the art.

d. DMSO: Sigma, St. Louis, Mo.

e. 96 Well ELISA Plate: Corning 96 Well Easy Wash, Modified flat Bottom Plate, Corning Cat. #25805-96.

f. NUNC 96-well V-bottom polypropylene plates for dilution of compounds: Applied Scientific Cat. #A-72092.

g. Vecastain ELITE ABC reagent: Vector, Burlingame, Calif.

h. Anti-src (327) mab: Schizosaccharomyces Pombe is used to express recombinant Src (Superti-Furga, et al., EMBO J., 12:2625-2634; Superti-Furga, et al., Nature Biochem., 14:600-605). S. Pombe strain SP200 (h-s leul.32 ura4 ade210) is grown as described and transformations are pRSP expression plasmids are done by the lithium acetate method (Superti-Furga, supra). Cells are grown in the presence of 1 μM thiamine to repress expression from the nmtl promoter or in the absence of thiamine to induce expression.

i. Monoclonal anti-phosphotyrosine, UBI 05-321 (UB40 may be used instead).

j. Turbo TMB-ELISA peroxidase substrate: Pierce Chemical.

Buffer Solutions

a. PBS (Dulbecco's Phosphate-Buffered Saline): GIBCO PBS, GIBCO Cat. #450-1300EB.

b. Blocking Buffer: 5% Non-fat milk (Carnation) in PBS.

c. Carbonate Buffer: Na₂CO₄ from Fischer, Cat. #S495, make up 100 mM stock solution.

d. Kinase Buffer: 1.0 ml (from 1M stock solution) MgCl₂; 0.2 ml (from a 1M stock solution) MnCl₂; 0.2 ml (from a 1M stock solution) DTT; 5.0 ml (from a 1M stock solution) HEPES; 0.1 ml TX-100; bring to 10 ml total volume with MilliQ H₂O.

e. Lysis Buffer: 5.0 HEPES (from 1M stock solution.); 2.74 ml NaCl (from 5M stock solution); 10 ml glycerol; 1.0 ml TX-100; 0.4 ml EDTA (from a 100 mM stock solution); 1.0 ml PMSF (from a 100 mM stock solution); 0.1 ml Na₃VO₄ (from a 0.1 M stock solution); bring to 100 ml total volume with MilliQ H₂O.

f. ATP: Sigma Cat. #A-7699, make up 10 mM stock solution (5.51 mg/ml).

g TRIS-HCl: Fischer Cat. #BP 152-5, to 600 ml MilliQ H₂O add 121.14 g material, adjust pH to 7.5 with HCl, bring to 1 L total volume with MilliQ H₂O.

h. NaCl: Fischer Cat. #S271-10, Make up 5M stock solution with MilliQ H₂O.

i. Na₃VO₄: Fischer Cat. #S454-50; to 80 ml MilliQ H₂O, add 1.8 g material; adjust pH to 10.0 with HCl or NaOH; boil in a microwave; cool; check pH, repeat pH adjustment until pH remains stable after heating/cooling cycle; bring to 100 ml total volume with MilliQ H₂O; make 1 ml aliquots and store at 80° C.

j. MgCl₂: Fischer Cat. #M33-500, make up 1M stock solution with MilliQ H₂O.

k. HEPES: Fischer Cat. #BP 310-500; to 200 ml MilliQ H₂O, add 59.6 g material, adjust pH to 7.5, bring to 250 ml total volume with MilliQ H₂O, sterile filter (1M stock solution).

l. TBST Buffer: TBST Buffer: To 900 ml dH₂O add 6.057 g TRIS and 8.766 g NaCl; adjust pH to 7.2 with HCl, add 1.0 ml Triton-X100; bring to 1 L total volume with dH₂O.

m. MnCl₂: Fischer Cat. #M87-100, make up 1M stock solution with MilliQ H₂O.

n. DTT: Fischer Cat. #BP172-5.

o. TBS (TRIS Buffered Saline): to 900 ml MilliQ H₂O add 6.057 g TRIS and 8.777 g NaCl; bring to 1 L total volume with MilliQ H₂O.

p. Kinase Reaction Mixture: Amount per assay plate (100 wells): 1.0 ml Kinase Buffer, 200 μg GST-ζ, bring to final volume of 8.0 ml with MilliQ H₂O.

q. Biotin labeled EEEYEEYEEEYEEEYEEEY: Make peptide stock solution (1 mM, 2.98 mg/ml) in water fresh just before use.

r. Vectastain ELITE ABC reagent: To prepare 14 ml of working reagent, add 1 drop of reagent A to 15 ml TBST and invert tube several times to mix. Then add 1 drop of reagent B. Put tube on orbital shaker at room temperature and mix for 30 minutes.

Procedures

Preparation of src Coated ELISA Plate.

1. Coat ELISA plate with 0.5 μg/well anti-src mab in 100 μl of pH 9.6 sodium carbonate buffer; hold at 4° C. overnight.

2. Wash wells once with PBS.

3. Block plate with 0.15 ml 5% milk in PBS for 30 min. at room temperature.

4. Wash plate 5×with PBS.

5. Add 10 μg/well of src transformed yeast lysates diluted in Lysis Buffer (0.1 ml total volume per well). (Amount of lysate may vary between batches.) Shake plate for 20 minutes at room temperature.

Preparation of Phosphotyrosine Antibody-Coated ELISA Plate.

1. 4G10 plate: coat 0.5 μg/well 4G10 in 100 μl PBS overnight at 4° C. and block with 150 μl of 5% milk in PBS for 30 minutes at room temperature.

Kinase Assay Procedure.

1. Remove unbound proteins from plates and wash plates 5×with PBS.

2. Add 0.08 ml Kinase Reaction Mixture per well (containing 10 μl of 10×Kinase Buffer and 10 μM (final concentration) biotin-EEEYEEYEEEYEEEYEEEY per well diluted in water.

3. Add 10 μl of compound diluted in water containing 10% DMSO and pre-incubate for 15 minutes at room temperature.

4. Start kinase reaction by adding 10 μl/well of 0.05 mM ATP in water (5 μM ATP final).

5. Shake ELISA plate for 15 min. at room temperature.

6. Stop kinase reaction by adding 10 μl of 0.5 M EDTA per well.

7. Transfer 90 μl supernatant to a blocked 4G10 coated ELISA plate.

8. Incubate for 30 min. while shaking at room temperature.

9. Wash plate 5×with TBST.

10. Incubate with Vectastain ELITE ABC reagent (100 μl/well) for 30 min. at room temperature.

11. Wash the wells 5×with TBST.

12. Develop with Turbo TMB.

Biochemical lck Assay

This assay is used to determine lck protein kinase activities measuring phosphorylation of GST-ζ as the readout.

Materials and Reagents

a. Yeast transformed with lck. Schizosaccharomyces Pombe is used to express recombinant Lck (Superti-Furga, et al., EMBO J, 12:2625-2634; Superti-Furga, et al., Nature Biotech., 14:600-605). S. Pombe strain SP200 (h-s leul.32 ura4 ade210) is grown as described and transformations with pRSP expression plasmids are done by the lithium acetate method (Superti-Furga, supra). Cells are grown in the presence of 1 μM thiamine to induce expression.

b. Cell lysates: Yeast cells expressing lck are pelleted, washed once in water, re-pelleted and stored frozen at −80° C. until use.

c. GST-ζ: DNA encoding for GST-ζ fusion protein for expression in bacteria obtained from Arthur Weiss of the Howard Hughes Medical Institute at the University of California, San Francisco. Transformed bacteria are grown overnight while shaking at 25° C. GST-ζ is purified by glutathione affinity chromatography, Pharmacia, Alameda, Calif.

d. DMSO: Sigma, St. Louis, Mo.

e. 96-Well ELISA plate: Corning 96 Well Easy Wash, Modified Flat Bottom Plate, Corning Cat. #25805-96.

f. NUNC 96-well V-bottom polypropylene plates for dilution of compounds: Applied Scientific Cat. #AS-72092.

g. Purified Rabbit anti-GST antiserum: Amrad Corporation (Australia) Cat. #90001605.

h. Goat anti-Rabbit-IgG-HRP: Amersham Cat. #V010301.

i. Sheep ant-mouse IgG (H+L): Jackson Labs Cat. #5215-005-003.

j. Anti-Lck (3A5) mab: Santa Cruz Biotechnology Cat #sc-433.

k. Monoclonal anti-phosphotyrosine UBI 05-321 (UB40 may be used instead).

Buffer Solutions

a. PBS (Dulbecco's Phosphate-Buffered Saline) 1×solution: GIBCO PBS, GIBCO Cat. #450-1300EB.

b. Blocking Buffer: 100 g. BSA, 12.1 g. TRIS (pH 7.5), 58.44 g NaCl, 10 ml Tween-20, bring up to 1 L total volume with MilliQ H₂O.

c. Carbonate Buffer: Na₂CO₄ from Fischer, Cat. #S495; make up 100 mM solution with MilliQ H₂O.

d. Kinase Buffer: 1.0 ml (from 1M stock solution) MgCl₂; 0.2 ml (from a 1M stock solution) MnCl₂; 0.2 ml (from a 1M stock solution) DTT; 5.0 ml (from a 1M stock solution) HEPES; 0.1 ml TX-100; bring to 10 ml total volume with MilliQ H₂O.

e. Lysis Buffer: 5.0 HEPES (from 1M stock solution.); 2.74 ml NaCl (from 5M stock solution); 10 ml glycerol; 1.0 ml TX-100; 0.4 ml EDTA (from a 100 mM stock solution); 1.0 ml PMSF (from a 100 mM stock solution); 0.1 ml Na₃VO₄ (from a 0.1 M stock solution); bring to 100 ml total volume with MilliQ H₂O.

f. ATP: Sigma Cat. #A-7699, make up 10 mM stock solution (5.51 mg/ml).

g TRIS-HCl: Fischer Cat. #BP 152-5, to 600 ml MilliQ H₂O add 121.14 g material, adjust pH to 7.5 with HCl, bring to 1 L total volume with MilliQ H₂O.

h. NaCl: Fischer Cat. #S271-10, Make up 5M stock solution with MilliQ H₂O.

i Na₃VO₄: Fischer Cat. #S454-50; to 80 ml MilliQ H₂O, add 1.8 g material; adjust pH to 10.0 with HCl or NaOH; boil in a microwave; cool; check pH, repeat pH adjustment until pH remains stable after heating/cooling cycle; bring to 100 ml total volume with MilliQ H₂O; make 1 ml aliquots and store at -80° C.

j. MgCl₂: Fischer Cat. #M33-500, make up 1M stock solution with MilliQ H₂O.

k. HEPES: Fischer Cat. #BP 310-500; to 200 ml MilliQ H₂O, add 59.6 g material, adjust pH to 7.5, bring to 250 ml total volume with MilliQ H₂O, sterile filter (1M stock solution).

l. Albumin, Bovine (BSA), Sigma Cat. #A4503; to 150 ml MilliQ H₂O add 30 g material, bring 300 ml total volume with MilliQ H₂O, filter through 0.22 μm filter, store at 4° C.

m. TBST Buffer: To 900 ml dH₂O add 6.057 g TRIS and 8.766 g NaCl; adjust pH to 7.2 with HCl, add 1.0 ml Triton-X100; bring to 1 L total volume with dH₂O.

n. MnCl2: Fischer Cat. #M87-100, make up 1M stock solution with MilliQ H₂O.

o. DTT: Fischer Cat. #BP172-5.

p. TBS (TRIS Buffered Saline): to 900 ml MilliQ H O add 6.057 g TRIS and 8.777 g NaCl; bring to 1 L total volume with MilliQ H₂O.

q Kinase Reaction Mixture: Amount per assay plate (100 wells): 1.0 ml Kinase Buffer, 200 μg GST-ζ, bring to final volume of 8.0 ml with MilliQ H₂O.

Procedures

Preparation of Lck Coated ELISA Plate

1. Coat 2.0 μg/well Sheep anti-mouse IgG in 100 μl of pH 9.6 sodium carbonate buffer at 4° C. overnight.

2. Wash well once with PBS.

3. Block plate with 0.15 ml of blocking Buffer for 30 min. at room temp.

4. Wash plate 5×with PBS.

5. Add 0.5 μg/well of anti-lck (mab 3A5) in 0.1 ml PBS at room temperature for 1-2 hours.

6. Wash plate 5×with PBS.

7. Add 20 μg/well of lck transformed yeast lysates diluted in Lysis Buffer (0.1 ml total volume per well). Shake plate at 4° C. overnight to prevent loss of activity.

Preparation of Phosphotyrosine Antibody-Coated ELISA Plate

1. UB40 plate: 1.0 μg/well UB40 in 100 μl of PBS overnight at 4° C. and block with 150 μl of Blocking Buffer for at least 1 hour.

Kinase Assay Procedure

1. Remove unbound proteins from plates and wash plates 5×with PBS.

2. Add 0.08 ml Kinase Reaction Mixture per well (containing 10 μl of 10×Kinase Buffer and 2 μg GST-ζ per well diluted with water).

3. Add 10 μl of compound diluted in water containing 10% DMSO and pre-incubate for 15 minutes at room temperature.

4. Start kinase reaction by adding 10 μl/well of 0.1 mM ATP in water (10 μM ATP final).

5. Shake ELISA plate for 60 min. at room temperature.

6. Stop kinase reaction by adding 10 μl of 0.5 M EDTA per well.

7. Transfer 90 μl supernatant to a blocked 4G10 coated ELISA plate from section B, above.

8. Incubate while shaking for 30 min. at room temperature.

9. Wash plate 5×with TBST.

10. Incubate with Rabbit anti-GST antibody at 1:5000 dilution in 100 μl TBST for 30 min. at room temperature.

11. Wash the wells 5×with TBST.

12. Incubate with Goat anti-Rabbit-IgG-HRP at 1:20,000 dilution in 100 μl of TBST for 30 min. at room temperature.

13. Wash the wells 5×with TBST.

14. Develop with Turbo TMB.

Assay Measuring Phosphorylating Function of RAF

The following assay reports the amount of RAF-catalyzed phosphorylation of its target protein MEK as well as MEK's target MAPK. The RAF gene sequence is described in Bonner et al., 1985, Molec. Cell. Biol., 5:1400-1407, and is readily accessible in multiple gene sequence data banks. Construction of the nucleic acid vector and cell lines utilized for this portion of the invention are fully described in Morrison et al., 1988, Proc. Natl. Acad. Sci. USA, 85:8855-8859.

Materials and Reagents

1. Sf9 (Spodoptera frugiperda) cells; GIBCO-BRL, Gaithersburg, Md.

2. RIPA buffer: 20 mM Tris/HCl pH 7.4, 137 mM NaCl, 10% glycerol, 1 mM PMSF, 5 mg/L Aprotenin, 0.5% Triton X-100;

3. Thioredoxin-MEK fusion protein (T-MEK): T-MEK expression and purification by affinity chromatography are performed according to the manufacturer's procedures. Catalog#K 350-01 and R 350-40, Invitrogen Corp., San Diego, Calif.

4. His-MAPK (ERK 2); His-tagged MAPK is expressed in XL1 Blue cells transformed with pUC18 vector encoding His-MAPK. His-MAPK is purified by Ni-affinity chromatography. Cat#27-4949-01, Pharmacia, Alameda, Calif., as described herein.

5. Sheep anti mouse IgG: Jackson laboratories, West Grove, Pa. Catalog, #515-006-008, Lot#28563

6. RAF-1 protein kinase specific antibody: URP2653 from UBI.

7. Coating buffer: PBS; phosphate buffered saline, GIBCO-BRL, Gaithersburg, Md.

8. Wash buffer: TBST (50 mM Tris/HCL pH 7.2, 150 mM NaCl, 0.1% Triton X-100).

9. Block buffer: TBST, 0.1% ethanolamine pH 7.4

10. DMSO, Sigma, St. Louis, Mo.

11. Kinase buffer (KB): 20 mM HEPES/HCl pH 7.2, 150 mM NaCl, 0.1% Triton X-100, 1 mM PMSF, 5 mg/L Aprotenin, 75 mM sodium orthovanadate, 0.5 MM DTT and 10 mM MgCl₂.

12. ATP mix: 100 mM MgCl₂, 300 mM ATP, 10 mCi γ³³P ATP (Dupont-NEN)/mL.

13 Stop solution: 1% phosphoric acid; Fisher, Pittsburgh, Pa.

14. Wallac Cellulose Phosphate Filter mats; Wallac, Turku, Finnland.

15. Filter wash solution: 1% phosphoric acid, Fisher, Pittsburgh, Pa.

16. Tomtec plate harvester, Wallac, Turku, Finnland. 17. Wallac beta plate reader #1205, Wallac, Turku, Finnland.

18. NUNC 96-well V bottom polypropylene plates for compounds Applied Scientific Catalog #AS-72092.

Procedure

All of the following steps are conducted at room temperature unless specifically indicated otherwise.

1. ELISA plate coating: ELISA wells are coated with 100 ml of Sheep anti mouse affinity purified antiserum (1 mg/100 mL coating buffer) over night at 4° C. ELISA plates can be used for two weeks when stored at 4° C.

2. Invert the plate and remove liquid. Add 100 mL of blocking solution and incubate for 30 min.

3. Remove blocking solution and wash four times with wash buffer. Pat the plate on a paper towel to remove excess liquid.

4. Add 1 mg of antibody specific for RAF-1 to each well and incubate for 1 hour. Wash as described in step 3.

5. Thaw lysates from RAS/RAF infected Sf9 cells and dilute with TBST to 10 mg/100 mL. Add 10 mg of diluted lysate to the wells and incubate for 1 hour. Shake the plate during incubation. Negative controls receive no lysate. Lysates from RAS/RAF infected Sf9 insect cells are prepared after cells are infected with recombinant baculoviruses at a MOI of 5 for each virus, and harvested 48 hours later. The cells are washed once with PBS and lysed in RIPA buffer. Insoluble material is removed by centrifugation (5 min at 10,000×g). Aliquots of lysates are frozen in dry ice/ethanol and stored at −80° C. until use.

6. Remove non-bound material and wash as outlined above (step 3).

7. Add 2 mg of T-MEK and 2 mg of His-MAEPK per well and adjust the volume to 40 ml with kinase buffer. Methods for purifying T-MEK and MAPK from cell extracts are provided herein by example.

8. Pre-dilute compounds (stock solution 10 mg/ml DMSO) or extracts 20 fold in TBST plus 1% DMSO. Add 5 ml of the pre-diluted compounds/extracts to the wells described in step 6. Incubate for 20 min. Controls receive no drug.

9. Start the kinase reaction by addition of 5 ml ATP mix; Shake the plates on an ELISA plate shaker during incubation.

10. Stop the kinase reaction after 60 min by addition of 30 mL stop solution to each well.

11. Place the phosphocellulose mat and the ELISA plate in the Tomtec plate harvester. Harvest and wash the filter with the filter wash solution according to the manufacturer's recommendation. Dry the filter mats. Seal the filter mats and place them in the holder. Insert the holder into radioactive detection apparatus and quantify the radioactive phosphorous on the filter mats.

Alternatively, 40 mL aliquots from individual wells of the assay plate can be transferred to the corresponding positions on the phosphocellulose filter mat. After air drying the filters, put the filters in a tray. Gently rock the tray, changing the wash solution at 15 min intervals for 1 hour. Air-dry the filter mats. Seal the filter mats and place them in a holder suitable for measuring the radioactive phosphorous in the samples. Insert the holder into a detection device and quantify the radioactive phosphorous on the filter mats.

CDK2/Cyclin A—Inhibition Assay

This assay analyzes the protein kinase activity of CDK2 in exogenous substrate.

Reagents

A. Buffer A: (80 mM Tris (pH 7.2), 40 mM MgCl₂), 4.84 g. Tris (F.W.=121.1 g/mol), 4.07 g. MgCl₂ (F.W.=203.31 g/mol) dissolved in 500 ml H₂O. Adjust pH to 7.2 with HCl.

B. Histone H1 solution (0.45 mg/ml Histone H1 and 20 mM HEPES pH 7.2: 5 mg Histone H1 (Boehinger Mannheim) in 11.111 ml 20 mM HEPES pH 7.2 (477 mg HEPES (F.W.=238.3 g/mol) dissolved in 100 ml ddH₂O, stored in 1 ml aliquots at −80° C.

C. ATP solution (60 μM ATP, 300 μg/ml BSA, 3 mM DTT): 120 μl 10 mM ATP, 600 μl 10 mg/ml BSA to 20 ml, stored in 1 ml aliquots at −80° C.

D. CDK2 solution: cdk2/cyclin A in 10 mM HEPES pH 7.2, 25 mM NaCl, 0.5 mM DTT, 10% glycerol, stored in 9 μl aliquots at

−80° C.

Protocol

1. Prepare solutions of inhibitors at three times the desired final assay concentration in ddH₂O/15% DMSO by volume.

2. Dispense 20 μl of inhibitors to wells of polypropylene 96-well plates (or 20 μl 15% DMSO for positive and negative controls).

3. Thaw Histone Hi solution (1 ml/plate), ATP solution (1 ml/plate plus 1 aliquot for negative control), and CDK2 solution (9 μl/plate). Keep CDK2 on ice until use. Aliquot CDK2 solution appropriately to avoid repeated freeze-thaw cycles.

4. Dilute 9 μl CDK2 solution into 2.1 ml Buffer A (per plate). Mix. Dispense 20 μl into each well.

5. Mix 1 ml Histone Hi solution with 1 ml ATP solution (per plate) into a 10 ml screw cap tube. Add γ³³P ATP to a concentration of 0.15 μCi/20 μl (0.15 μCi/well in assay). Mix carefully to avoid BSA frothing. Add 20 μl to appropriate wells. Mix plates on plate shaker. For negative control, mix ATP solution with an equal amount of 20 mM HEPES pH 7.2 and add γ³³P ATP to a concentration of 0.15 μCi/20 μl solution. Add 20 μl to appropriate wells.

6. Let reactions proceed for 60 minutes.

7. Add 35 μl 10% TCA to each well. Mix plates on plate shaker.

8. Spot 40 μl of each sample onto P30 filter mat squares. Allow mats to dry (approx. 10-20 minutes).

9 Wash filter mats 4×10 minutes with 250 ml 1% phosphoric acid (10 ml phosphoric acid per liter ddH₂O).

10. Count filter mats with beta plate reader.

Cellular/Biologic Assays

PDGF-Induced BrdU Incorporation Assay

Materials and Reagents

(1) PDGF: human PDGF B/B; 1276-956, Boehringer Mannheim, Germany.

(2) BrdU Labeling Reagent: 10 mM, in PBS (pH 7.4), Cat. No. 1 647 229, Boehringer Mannheim, Germany.

(3) FixDenat: fixation solution (ready to use), Cat. No. 1 647 229, Boehringer Mannheim, Germany.

(4) Anti-BrdU-POD: mouse monoclonal antibody conjugated with peroxidase, Cat. No. 1 647 229, Boehringer Mannheim, Germany.

(5) TMB Substrate Solution: tetramethylbenzidine (TMB), ready to use, Cat. No. 1 647 229, Boehringer Mannheim, Germany.

(6) PBS Washing Solution: 1×PBS, pH 7.4 (Sugen, Inc., Redwood City, Calif.).

(7) Albumin, Bovine (BSA): fraction V powder; A-8551, Sigma Chemical Co., USA.

(8) 3T3 cell line genetically engineered to express human PDGF-R.

Protocol

(1) Cells are seeded at 8000 cells/well in DMEM, 10% CS, 2 mM Gln in a 96 well plate. Cells are incubated overnight at 37° C. in 5% CO₂.

(2) After 24 hours, the cells are washed with PBS, and then are serum starved in serum free medium (0%CS DMEM with 0.1% BSA) for 24 hours.

(3) On day 3, ligand (PDGF, 3.8 nM, prepared in DMEM with 0.1% BSA) and test compounds are added to the cells simultaneously. The negative control wells receive serum free DMEM with 0.1% BSA only; the positive control cells receive the ligand (PDGF) but no test compound. Test compounds are prepared in serum free DMEM with ligand in a 96 well plate, and serially diluted for 7 test concentrations.

(4) After 20 hours of ligand activation, diluted BrdU labeling reagent (1:100 in DMEM, 0.1% BSA) is added and the cells are incubated with BrdU (final concentration=10 μM) for 1.5 hours.

(5) After incubation with labeling reagent, the medium is removed by decanting and tapping the inverted plate on a paper towel. FixDenat solution is added (50 μl/well) and the plates are incubated at room temperature for 45 minutes on a plate shaker.

(6) The FixDenat solution is thoroughly removed by decanting and tapping the inverted plate on a paper towel. Milk is added (5% dehydrated milk in PBS, 200 μl/well) as a blocking solution and the plate is incubated for 30 minutes at room temperature on a plate shaker.

(7) The blocking solution is removed by decanting and the wells are washed once with PBS. Anti-BrdU-POD solution (1:100 dilution in PBS, 1% BSA) is added (100 μl/well) and the plate is incubated for 90 minutes at room temperature on a plate shaker.

(8) The antibody conjugate is thoroughly removed by decanting and rinsing the wells 5 times with PBS, and the plate is dried by inverting and tapping on a paper towel.

(9) TMB substrate solution is added (100 μl/well) and incubated for 20 minutes at room temperature on a plate shaker until color development is sufficient for photometric detection.

(10) The absorbance of the samples are measured at 410 nm (in “dual wavelength” mode with a filter reading at 490 nm, as a reference wavelength) on a Dynatech ELISA plate reader.

EGF-Induced BrdU Incorporation Assay

Materials and Reagents

(1) EGF: mouse EGF, 201; Toyobo, Co., Ltd. Japan.

(2) BrdU Labeling Reagent: 10 mM, in PBS (pH 7.4), Cat. No. 1 647 229, Boehringer Mannheim, Germany.

(3) FixDenat: fixation solution (ready to use), Cat. No. 1 647 229, Boehringer Mannheim, Germany.

(4) Anti-BrdU-POD: mouse monoclonal antibody conjugated with peroxidase, Cat. No. 1 647 229, Boehringer Mannheim, Germany.

(5) TMB Substrate Solution: tetramethylbenzidine (TMB), ready to use, Cat. No. 1 647 229, Boehringer Mannheim, Germany.

(6) PBS Washing Solution: 1×PBS, pH 7.4 (Sugen, Inc., Redwood City, Calif.).

(7) Albumin, Bovine (BSA): fraction V powder; A-8551, Sigma Chemical Co., USA.

(8) 3T3 cell line genetically engineered to express human EGF-R.

Protocol

(1) Cells are seeded at 8000 cells/well in 10% CS, 2 mM Gln in DMEM, in a 96 well plate. Cells are incubated overnight at 37° C. in 5% CO₂.

(2) After 24 hours, the cells are washed with PBS, and then are serum starved in serum free medium (0% CS DMEM with 0.1% BSA) for 24 hours.

(3) On day 3, ligand (EGF, 2 nM, prepared in DMEM with 0.1% BSA) and test compounds are added to the cells simultaneously. The negative control wells receive serum free DMEM with 0.1% BSA only; the positive control cells receive the ligand (EGF) but no test compound. Test compounds are prepared in serum free DMEM with ligand in a 96 well plate, and serially diluted for 7 test concentrations.

(4) After 20 hours of ligand activation, diluted BrdU labeling reagent (1:100 in DMEM, 0.1% BSA) is added and the cells are incubated with BrdU (final concentration=10 μM) for 1.5 hours.

(5) After incubation with labeling reagent, the medium is removed by decanting and tapping the inverted plate on a paper towel. FixDenat solution is added (50 μl/well) and the plates are incubated at room temperature for 45 minutes on a plate shaker.

(6) The FixDenat solution is thoroughly removed by decanting and tapping the inverted plate on a paper towel. Milk is added (5% dehydrated milk in PBS, 200 μl/well) as a blocking solution and the plate is incubated for 30 minutes at room temperature on a plate shaker.

(7) The blocking solution is removed by decanting and the wells are washed once with PBS. Anti-BrdU-POD solution (1:100 dilution in PBS, 1% BSA) is added (100 μl/well) and the plate is incubated for 90 minutes at room temperature on a plate shaker.

(8) The antibody conjugate is thoroughly removed by decanting and rinsing the wells 5 times with PBS, and the plate is dried by inverting and tapping on a paper towel.

(9) TMB substrate solution is added (100 μl/well) and incubated for 20 minutes at room temperature on a plate shaker until color development is sufficient for photometric detection.

(10) The absorbance of the samples are measured at 410 nm (in “dual wavelength” mode with a filter reading at 490 nm, as a reference wavelength) on a Dynatech ELISA plate reader.

EGF-Induced Her2-Driven BrdU Incorporation

Materials and Reagents

(1) EGF: mouse EGF, 201; Toyobo, Co., Ltd. Japan

(2) BrdU Labeling Reagent: 10 mM, in PBS (pH 7.4), Cat. No. 1 647 229, Boehringer Mannheim, Germany.

(3) FixDenat: fixation solution (ready to use), Cat. No. 1 647 229, Boehringer Mannheim, Germany.

(4) Anti-BrdU-POD: mouse monoclonal antibody conjugated with peroxidase, Cat. No. 1 647 229, Boehringer Mannheim, Germany.

(5) TMB Substrate Solution: tetramethylbenzidine (TMB), ready to use, Cat. No. 1 647 229, Boehringer Mannheim, Germany.

(6) PBS Washing Solution: 1×PBS, pH 7.4, made in house.

(7) Albumin, Bovine (BSA): fraction V powder; A-8551, Sigma Chemical Co., USA.

(8) 3T3 cell line engineered to express a chimeric receptor having the extra-cellular domain of EGF-R and the intra-cellular domain of Her2.

Protocol

(1) Cells are seeded at 8000 cells/well in DMEM, 10% CS, 2 mM Gln in a 96-well plate. Cells are incubated overnight at 37° C. in 5% CO₂.

(2) After 24 hours, the cells are washed with PBS, and then are serum starved in serum free medium (0% CS DMEM with 0.1% BSA) for 24 hours.

(3) On day 3, ligand (EGF=2 nM, prepared in DMEM with 0.1% BSA) and test compounds are added to the cells simultaneously. The negative control wells receive serum free DMEM with 0.1% BSA only; the positive control cells receive the ligand (EGF) but no test compound. Test compounds are prepared in serum free DMEM with ligand in a 96 well plate, and serially diluted for 7 test concentrations.

(4) After 20 hours of ligand activation, diluted BrdU labeling reagent (1:100 in DMEM, 0.1% BSA) is added and the cells are incubated with BrdU (final concentration=10 μM) for 1.5 hours.

(5) After incubation with labeling reagent, the medium is removed by decanting and tapping the inverted plate on a paper towel. FixDenat solution is added (50 μl/well) and the plates are incubated at room temperature for 45 minutes on a plate shaker.

(6) The FixDenat solution is thoroughly removed by decanting and tapping the inverted plate on a paper towel. Milk is added (5% dehydrated milk in PBS, 200 μl/well) as a blocking solution and the plate is incubated for 30 minutes at room temperature on a plate shaker.

(7) The blocking solution is removed by decanting and the wells are washed once with PBS. Anti-BrdU-POD solution (1:100 dilution in PBS, 1% BSA) is added (100 μl/well) and the plate is incubated for 90 minutes at room temperature on a plate shaker.

(8) The antibody conjugate is thoroughly removed by decanting and rinsing the wells 5 times with PBS, and the plate is dried by inverting and tapping on a paper towel.

(9) TMB substrate solution is added (100 μl/well) and incubated for 20 minutes at room temperature on a plate shaker until color development is sufficient for photometric detection.

(10) The absorbance of the samples are measured at 410 nm (in “dual wavelength” mode with a filter reading at 490 nm, as a reference wavelength) on a Dynatech ELISA plate reader.

IGF1-Induced BrdU Incorporation Assay

Materials and Reagents

(1) IGF1 Ligand: human, recombinant; G511, Promega Corp, USA.

(2) BrdU Labeling Reagent: 10 mM, in PBS (pH 7.4), Cat. No. 1 647 229, Boehringer Mannheim, Germany.

(3) FixDenat: fixation solution (ready to use), Cat. No. 1 647 229, Boehringer Mannheim, Germany.

(4) Anti-BrdU-POD: mouse monoclonal antibody conjugated with peroxidase, Cat. No. 1 647 229, Boehringer Mannheim, Germany.

(5) TMB Substrate Solution: tetramethylbenzidine (TMB), ready to use, Cat. No. 1 647 229, Boehringer Mannheim, Germany.

(6) PBS Washing Solution: 1×PBS, pH 7.4 (Sugen, Inc., Redwood City, Calif.).

(7) Albumin, Bovine (BSA): fraction V powder; A-8551, Sigma Chemical Co., USA.

(8) 3T3 cell line genetically engineered to express human IGF-1 receptor.

Protocol

(1) Cells are seeded at 8000 cells/well in DMEM, 10% CS, 2 mM Gln in a 96-well plate. Cells are incubated overnight at 37° C. in 5% CO₂.

(2) After 24 hours, the cells are washed with PBS, and then are serum starved in serum free medium (0%CS DMEM with 0.1% BSA) for 24 hours.

(3) On day 3, ligand (IGF1=3.3 nM, prepared in DMEM with 0.1% BSA) and test compounds are added to the cells simultaneously. The negative control wells receive serum free DMEM with 0.1% BSA only; the positive control cells receive the ligand (IGF1) but no test compound. Test compounds are prepared in serum free DMEM with ligand in a 96 well plate, and serially diluted for 7 test concentrations.

(4) After 16 hours of ligand activation, diluted BrdU labeling reagent (1:100 in DMEM, 0.1% BSA) is added and the cells are incubated with BrdU (final concentration=10 μM) for 1.5 hours.

(5) After incubation with labeling reagent, the medium is removed by decanting and tapping the inverted plate on a paper towel. FixDenat solution is added (50 μl/well) and the plates are incubated at room temperature for 45 minutes on a plate shaker.

(6) The FixDenat solution is thoroughly removed by decanting and tapping the inverted plate on a paper towel. Milk is added (5% dehydrated milk in PBS, 200 μl/well) as a blocking solution and the plate is incubated for 30 minutes at room temperature on a plate shaker.

(7) The blocking solution is removed by decanting and the wells are washed once with PBS. Anti-BrdU-POD solution (1:100 dilution in PBS, 1% BSA) is added (100 μl/well) and the plate is incubated for 90 minutes at room temperature on a plate shaker.

(8) The antibody conjugate is thoroughly removed by decanting and rinsing the wells 5 times with PBS, and the plate is dried by inverting and tapping on a paper towel.

(9) TMB substrate solution is added (100 μl/well) and incubated for 20 minutes at room temperature on a plate shaker until color development is sufficient for photometric detection.

(10) The absorbance of the samples are measured at 410 nm (in “dual wavelength” mode with a filter reading at 490 nm, as a reference wavelength) on a Dynatech ELISA plate reader.

FGF-Induced BrdU Incorporation Assay

This assay measures FGF-induced DNA synthesis in 3Tc7/EGFr cells that express endogenous FGF receptors.

Materials and Reagents

1. FGF: human FGF2/bFGF (Gibco BRL, No. 13256-029).

2. BrdU Labeling reagent, (10 mM PBS (pH 7.4), Boehringer Mannheim Cat No. 1 647 229).

3. Fixdenat fixation solution (Boehringer Mannheim Cat No. 1 647 229).

4. Anti-BrdU-POD (mouse monoclonal antibody conjugated with peroxidase, Boehringer Mannheim Cat. No. 1 647 229).

5. TMB (tetramethylbenzidine, Boehringer Mannheim Cat. No. 1 647 229).

6. PBS washing solution, pH 7.4 (Sugen, Inc.).

7. Albumin, bovine (BSA), fraction V powder (Sigma Chemical Co., Cat. No. A-8551)

Procedure

1. 3T3 engineered cell line: 3T3c7/EGFr.

2. Cells are seeded at 8,000 cells/well in DMEM, 10% CS and 2 mM Gln in a 96-well plate. Incubate 24 hours at 37° C. in 5% CO₂.

3. After 24 hours, wash cells with PBS then serum starve in serum free medium (0% DMEM, 0.1% BSA) for 24 hours.

4. Add ligand (FGF2 (1.5 nM in DMEM with 0.1% BSA) and test compound simultaneously. Negative control wells receive serum free DMEM with 0.1% BSA only; positive control wells receive FGF2 ligand but no test compound. Test compounds are prepared in serum-free DMEM with ligand in a 96-well plate and serially diluted to make seven (7) test concentrations.

5. After 20 hours, add diluted BrdU labeling reagent (1:100 BrdU:DMEM, 0.1% BSA, final concentration is 10 μM) to the cells and incubate for 1.5 hours.

6. Decant medium. Remove traces of material with paper towel. Add FixDenat (50 μl/well) and incubate at room temperature for 45 minutes on a plate shaker.

7. Remove Fixdenat solution. Add blocking solution (5% dehydrated milk in PBS (200 μl/well)) and incubate for 30 minutes at room temperature on a plate shaker.

8. Decant blocking solution; wash wells once with PBS. Add anti-BrdU-POD solution (1:100 dilution in PBS, 0.1% BSA); incubate for 90 minutes at room temperature on a plate shaker.

9. Decant antibody conjugate; rinse wells 5 times with PBS. Dry plate by inverting on paper towel and tapping.

10. Add TMB solution (100 μl/well); incubate 20 minutes at room temperature on a plate shaker until color development is sufficient for photometric detection.

11. Measure absorbance at 410 nM on a Dynatech ELISA plate reader using “Dual wavelength” mode with a filter at 490 nM.

Biochemical EGFR Assay

This assay measures the in vitro kinase activity of EGFR using ELISA.

Materials and Reagents

1. Corning 96-well Elisa plates (Corning Catalog No. 25805-96).

2. SUMO1 monoclonal anti-EGFR antibody (Biochemistry Lab, SUGEN, Inc.).

3. PBS (Dulbecco's Phosphate-Buffered Saline, Gibco Catalog No. 450-1300EB).

4. TBST Buffer

| | | Working | Amount |
|---|---|---|---|
| Reagent | M.W. | Concentration | per L |
| Tris | 121.14 | 50 mM | 6.057 g |
| NaCl | 58.44 | 150 mM | 8.766 g |
| Triton X-100 | NA | 0.1% | 1.0 ml |

5. Blocking Buffer:

| | | Working | Amount per |
|---|---|---|---|
| Reagent | M.W. | Concentration | 100 ml |
| Carnation Instant | | 5% | 5.0 g |
| Non-Fat Milk | | | |
| PBS | NA | NA | 100 ml |

6. A431 cell lysate (Screening Lab, SUGEN, Inc.)

7. TBS Buffer:

| | | Working | Amount |
|---|---|---|---|
| Reagent | M.W. | Concentration | per L |
| Tris | 121.14 | 50 mM | 6.057 g |
| NaCl | 58.44 | 150 mM | 8.766 g |

8. TBS+10% DMSO

| | | Working | Amount |
|---|---|---|---|
| Reagent | M.W. | Concentration | per L |
| Tris | 121.14 | 50 mM | 1.514 g |
| NaCl | 58.44 | 150 mM | 2.192 g |
| DMSO | NA | 10% | 25 ml |

9. Adenosine-5′-triphosphate (ATP, from Equine muscle, Sigma Cat. No. A-5394).

Prepare a 1.0 mM solution in dH₂O. This reagent should be made up immediately prior to use and kept on ice.

10. MnCl₂.

Prepare a 1.0 M stock solution in dH₂O.

11. ATP/MnCl₂ phosphorylation mix

| | Stock | Amount | Working |
|---|---|---|---|
| Reagent | solution | per 10 ml | Concentration |
| ATP | 1.0 mM | 300 μl | 30 μM |
| MnCl₂ | 1.0 M | 500 μl | 50 mM |
| dH₂O | | | 9.2 ml |

This reagent should be prepared immediately before use and kept on ice

12. NUNC 96-well V bottom polypropylene plates (Applied Scientific Cat. No. AS-72092).

13. Ethylenediaminetetraacetic acid (EDTA)

Prepare 200 mM working solution in dH₂O. Adjust to pH 8.0 with 10 N NaOH.

14. Rabbit polyclonal anti-phosphotyrosine serum (Biochemistry Lab, SUGEN, Inc.)

15. Goat anti-rabbit IgG peroxidase conjugate (Biosource Cat. No. ALI0404)

16. ABTS (2,2′-azino-bis(3-ethylbenzthiazoline-6-sulfonic acid), Sigma Cat. No. A-1888).

| | | Working | Amount |
|---|---|---|---|
| Reagent | M.W. | Concentration | per L |
| Citric Acid | 192.12 | 100 mM | 19.21 g |
| Na2HP04 | 141.96 | 250 mM | 35.49 g |
| ABTS | NA | 0.5 mg/ml | 500 mg |

Mix first two ingredients in about 900 ml dH₂O, adjust pH to 4.0 with phosphoric acid. Add ABTS, cover, let sit about 0.5 hr., filter. The solution should be kept in the dark at 4° C. until ready to use.

17. Hydrogen peroxide 30% solution (Fisher Cat. No. H325)

18. ABTS/H₂O₂

Mix 15 ml ABTS solution and 2.0 μl H₂O₂. Prepare 5 minutes before use.

19. 0.2 M HCl

Procedure

1. Coat Corning 96 well ELISA plates with 0.5 μg SUMO1 in 100 μl PBS per well, store overnight at 4° C.

2. Remove unbound SUMO1 from wells by inverting plate to remove liquid. Wash 1×with dH₂O. Pat the plate on a paper towel to remove excess liquid.

3. Add 150 μl of Blocking Buffer to each well. Incubate for 30 min. at room temperature with shaking.

4. Wash plate 3×with deionized water, then once with TBST. Pat plate on a paper towel to remove excess liquid and bubbles.

5. Dilute lysate in PBS (7 μg lysate/100 μl PBS).

6. Add 100 μl of diluted lysate to each well. Shake at room temperature for 60 min.

7. Wash plates as described in 4, above.

8. Add 120 μl TBS to ELISA plate containing captured EGFR.

9. Dilute test compound 1:10 in TBS in 96-well polypropylene plates (ie. 10 μl compound+90 μl TBS).

10. Add 13.5 μl diluted test compound to ELISA plate. To control wells (wells which do not receive any test compound), add 13.5 μl TBS+10% DMSO.

11. Incubate for 30 minutes while shaking at room temperature.

12. Add 15 μl phosphorylation mix directly to all wells except negative control well which does not receive ATP/MnCl₂ (final well volume should be approximately 150 μl with 3 μM ATP/5 mM MnCl₂ final concentration in each well.) Incubate 5 minutes while shaking.

13. After 5 minutes, stop reaction by adding 16.5 μl of 200 mM EDTA (pH 8.0) to each well, shaking continuously. After the EDTA has been added, shake for 1 min.

14. Wash 4×with deionized water, twice with TBST.

15. Add 100 μl anti-phosphotyrosine (1:3000 dilution in TBST) per well. Incubate 30-45 min. at room temperature, with shaking.

16. Wash as described in 4, above.

17. Add 100 μl Biosource Goat anti-rabbit IgG peroxidase conjugate (1:2000 dilution in TBST) to each well. Incubate 30 min. at room temperature, with shaking.

18. Wash as described in 4, above.

19. Add 100 μl of ABTS/H₂O₂ solution to each well.

20. Incubate 5 to 10 minutes with shaking. Remove any bubbles.

21. If necessary stop reaction with the addition of 100 μl 0.2 M HCl per well.

22. Read assay on Dynatech MR7000 ELISA reader. Test Filter: 410 nM Reference Filter: 630 Nm.

Biochemical PDGFR Assay

This assay measures the in vitro kinase activity of PDGFR using ELISA.

Materials and Reagents

Unless otherwise noted, the preparation of working solution of the following reagents is the same as that for the Biochemical EGFR assay, above.

1. Corning 96-well Elisa plates (Corning Catalog No. 25805-96).

2. 28D4C10 monoclonal anti-PDGFR antibody (Biochemistry Lab, SUGEN, Inc.).

3. PBS (Dulbecco's Phosphate-Buffered Saline, Gibco Catalog No. 450-1300EB)

4. TBST Buffer.

5. Blocking Buffer.

6. PDGFR-p expressing NIH 3T3 cell lysate (Screening Lab, SUGEN, Inc.).

7. TBS Buffer.

8. TBS+10% DMSO.

9. Adenosine-5′-triphosphate (ATP, from Equine muscle, Sigma Cat. No. A-5394).

10. MnCl₂.

11. Kinase buffer phosphorylation mix.

| | Stock | Amount | Working |
|---|---|---|---|
| Reagent | solution | per 10 ml | Concentration |
| Tris | 1 M | 250 μl | 25 mM |
| NaCl | 5 M | 200 μl | 100 mM |
| MnCl₂ | 1 M | 100 μl | 10 mM |
| TX-100 | 100 mM | 50 μl | 0.5 mM |

12. NUNC 96-well V bottom polypropylene plates (Applied Scientific Cat. No. AS-72092).

13. Ethylenediaminetetraacetic acid (EDTA).

14. Rabbit polyclonal anti-phosphotyrosine serum (Biochemistry Lab, SUGEN, Inc.).

15. Goat anti-rabbit IgG peroxidase conjugate (Biosource Cat. No. ALI0404).

16. 2,2′-azino-bis(3-ethylbenzthiazoline-6-sulfonic acid) (ABTS, Sigma Cat. No. A-1888).

17. Hydrogen peroxide 30% solution (Fisher Cat. No. H325).

18. ABTS/H₂O₂.

19. 0.2 M HCl.

Procedure

1. Coat Corning 96 well ELISA plates with 0.5 μg 28D4C10 in 100 μl PBS per well, store overnight at 4° C.

2. Remove unbound 28D4C10 from wells by inverting plate to remove liquid. Wash 1×with dH₂O. Pat the plate on a paper towel to remove excess liquid.

3. Add 150 μl of Blocking Buffer to each well. Incubate for 30 min. at room temperature with shaking.

4. Wash plate 3×with deionized water, then once with TBST. Pat plate on a paper towel to remove excess liquid and bubbles.

5. Dilute lysate in HNTG (10 μg lysate/100 μl HNTG)

6. Add 100 μl of diluted lysate to each well. Shake at room temperature for 60 min.

7. Wash plates as described in 4, above.

8. Add 80 μl working kinase buffer mix to ELISA plate containing captured PDGFR.

9. Dilute test compound 1:10 in TBS in 96-well polypropylene plates (i.e., 10 μl compound+90 μl TBS).

10. Add 10 μl diluted test compound to ELISA plate. To control wells (wells which do not receive any test compound), add 10 μl TBS+10% DMSO.

11. Incubate for 30 minutes while shaking at room temperature.

12. Add 10 μl ATP directly to all wells except negative control well (final well volume should be approximately 100 μl with 20 μM ATP in each well.) Incubate 30 minutes while shaking.

13. After 30 minutes, stop reaction by adding 10 μl of 200 mM EDTA (pH 8.0) to each well.

14. Wash 4×with deionized water, twice with TBST.

15. Add 100 μl anti-phosphotyrosine (1:3000 dilution in TBST) per well. Incubate 30-45 min. at room temperature, with shaking.

16. Wash as described in 4, above.

17. Add 100 μl Biosource Goat anti-rabbit IgG peroxidase conjugate (1:2000 dilution in TBST) to each well. Incubate 30 min. at room temperature, with shaking.

18. Wash as described in 4, above.

19. Add 100 μl of ABTS/H₂O₂ solution to each well.

20. Incubate 10 to 30 minutes with shaking. Remove any bubbles.

21. If necessary stop reaction with the addition of 100 μl 0.2 M HCl per well.

22. Read assay on Dynatech MR7000 ELISA reader: test filter: 410 nM, reference filter: 630 nM.

Biochemical FGFR Assay

This assay measures in vitro kinase activity of the Myc-GyrB-FGFR fusion protein using ELISA.

Materials and Reagents

1. HNTG

| | | 5x Stock | Amount | 1x Working |
|---|---|---|---|---|
| Reagent | M.W. | Concentration | per L | Concentration |
| HERES | 238.3 | 100 mM | 23.83 g | 20 mM |
| NaCl | 58.44 | 750 mM | 43.83 g | 150 mM |
| Glycerol | NA | 50% | 500 ml | 10% |
| Triton X-l00 | NA | 5% | 10 ml | 1.0% |

To make a liter of 5×stock solution, dissolve HEPES and NaCl in about 350 ml dH₂O, adjust pH to 7.2 with HCl or NaOH (depending on the HEPES that is used), add glycerol, Triton X-100 and then dH₂O to volume.

2. PBS (Dulbecco's Phosphate-Buffered Saline, Gibco Catalog #450-1300EB).

3. Blocking Buffer.

4. Kinase Buffer.

| | | 10x Stock | 1x Working |
|---|---|---|---|
| Reagent | M.W. | Concentration | Concentration |
| HEPES (pH 7.2) | 238.3 | 500 mM | 50 mM |
| MnCl₂ | | 20 mM | 2 mM |
| MgCl₂ | 203.32 | 200 mM | 10 mM |
| Triton-X-100 | | 1% | 0.1% |
| DTT | 380.35 | 5 mM | 0.5 mM |

5. Phenylmethylsulfonyl fluoride (PMSF, Sigma, Cat. No. P-7626):

Working solution: 100 mM in ethanol.

6. ATP (Bacterial source, Sigma Cat. No. A-7699)

Use 3.31 mg per ml MilliQ H2O for a stock concentration of 6 mM.

7. Biotin conjugated anti-phosphotyrosine mab (clone 4G10, Upstate Biotechnology Inc. Cat. No. 16-103, Ser. No. 14495).

8. Vectastain Elite ABC reagent (Avidin peroxidase conjugate, Vector Laboratories Cat. No. PK-6 100).

9. ABTS Solution.

10. Hydrogen peroxide 30% solution (Fisher Catalog # H325).

11. ABTS/H₂O₂

12. 0.2 M HCl.

13. TRIS HCl (Fischer Cat. No. BP 152-5).

Prepare 1.0 mM solution in MilliQ H₂O, adjust pH to 7.2 with HCl.

14. NaCl (Fisher Cat. No. S271-10).

Prepare 5 M solution in MilliQ H₂O.

15. MgCl₂ (Fisher Cat. No. M33-500)

Prepare 1 M solution in MilliQ H₂O.

16. HEPES (Fisher Cat. No. BP310-500).

Prepare 1 M solution in MilliQ H₂O, adjust pH to 7.5, sterile filter.

17. TBST Buffer.

18. Sodium Carbonate Buffer (Fisher Cat. No. S495).

Prepare 0.1 M solution in MilliQ H2O, adjust pH to 9.6 with NaOH, filter.

19. Dithiothreitol (DTT, Fisher Cat. No. BP172-25).

Prepare 0.5 mM working solution in MilliQ H₂O just prior to use. Store at −200 C until used, discard any leftover.

20. MnCl₂.

21. Triton X-100.

22. Goat α-Rabbit IgG (Cappel).

23. Affinity purified Rabbit α GST GyrB (Biochemistry Lab. SUGEN, Inc.).

Procedure

All of the following steps are conducted at room temperature unless otherwise indicated.

1. Coat Corning 96-well ELISA plates with 2 μg Goat α-Rabbit antibody per well in Carbonate Buffer such that total well volume is 100 μl. Store overnight at 4° C.

2. Remove unbound Goat a-Rabbit antibody by inverting plate to remove liquid. Pat plate on a paper towel to remove excess liquid and bubbles

3. Add 150 μl Blocking Buffer (5% Low Fat Milk in PBS) to each well. Incubate while shaking on a micro-titer plate shaker for 30 min.

4. Wash 4×with TBST. Pat plate on a paper towel to remove excess liquid and bubbles.

5. Add 0.5 μg Rabbit a-GyrB antibody per well. Dilute antibody in DPBS to a final volume of 100 μl per well. Incubate with shaking on a micro-titer plate shaker at room temperature for 1 hour.

6. Wash 4×with TBST as described in step 4.

7. Add 2 μg COS/FGFR cell lysate (Myc-GyrB-FGFR source) in HNTG to each well to give a final volume of 100 μl per well. Incubate with shaking on a micro-titer plate shaker for 1 hour.

8. Wash 4×with TEST as described in step 4.

9. Add 80 μl of 1×kinase buffer per well.

10. Dilute test compound 1:10 in 1×kinase buffer+1% DMSO in a polypropylene 96 well plate.

11. Transfer 10 μl of diluted test compound solution and control wells from polypropylene plate wells to the corresponding ELISA plate wells, incubate with shaking on a micro-titer plate shaker for 20 minutes.

12. Add 10 μl of 70 μM ATP diluted in kinase buffer to positive control and test wells (Final ATP concentration is 7 μM/well). Add 10 μl 1×kinase buffer to negative control wells. Incubate with shaking on a micro-titer plate shaker for 15 min.

13. Stop kinase reaction by adding 5 μl 0.5 M EDTA to all wells.

14. Wash 4×with TBST as described in step 4.

15. Add 100 μl biotin conjugated α-phosphotyrosine mab (b4G10) diluted in TBST to each well. Incubate with shaking on a micro-titer plate shaker for 30 minutes.

16. Prepare Vectastain ABC reagent. Add 1 drop reagent A to 15 ml TBST. Mix by inverting tube several times. Add 1 drop reagent B and mix again.

17. Wash 4×with TBST as described in step 4.

18. Add 100 μl ABC HRP reagent to each well. Incubate with shaking on a micro-titer plate shaker for 30 minutes.

19. Wash 4×with TEST as described in step 4.

20. Add 100 μl of ABTS/H₂O₂ solution to each well.

22. Incubate 5 to 15 minutes with shaking. Remove any bubbles.

23. If necessary stop reaction by adding 1 00 μl of 0.2M HCl/well.

24. Read assay on Dynatech MR7000 ELISA Plate Reader; test filter: 410 nM, reference filter: 630 nM.

Biochemical FLK-1 Assay

This assay evaluates flk-1 autophosphorylation activity in vitro using ELISA.

Materials and Reagents

1. 15 cm tissue culture dishes

2. Flk-1/NIH cells: NIH fibroblast line over-expressing human flk-1 clone 3 (SUGEN, Inc., obtained from MPI, Martinsried, Germany).

3. Growth medium: DMEM plus heat inactivated 10% FBS and 2 mM Glutamine (Gibco-BRL).

4. Starvation medium: DMEM plus 0.5% heat-inactivated FBS, 2 mM Glutamine (Gibco-BRL).

5. Corning 96-well ELISA plates (Corning Cat. No. 25805-96).

6. L4 or E38 monoclonal antibody specific for flk-1; Purified by Protein-A agarose affinity chromatography (SUGEN, Inc.).

7. PBS (Dulbecco's Phosphate-Buffered Saline) Gibco Cat. No. 450-1300EB).

8. HNTG (see BIOCHEMICAL FGFR for preparation).

9. Pierce BCA protein determination kit.

10. Blocking buffer

11. TBST (pH 7.0)

12. Kinase Buffer

13. Kinase Stop Solution: 200 mM EDTA.

14. Biotinylated 4G10, specific for phosphotyrosine (UBI, Cat. No. No. 16-103).

15. AB kit (Vector Laboratories Cat. No. PK 4000).

16. DMSO

17. NUNC 96-well V bottom polypropylene plates (Applied Scientific Cat. No. AS-72092).

18. Turbo-TMB (Pierce).

19. Turbo-TMB stop solution: 1 M H₂SO₄.

20. ATP (Sigma Cat. No. A-7699).

21. 20% DMSO in TBS (pH 7.0).

Procedure

Cell Growth and Lysate Preparation

1. Seed cell into growth medium and grow for 2-3 days to 90-100% confluency at 37° C. and 5% CO₂. Do not exceed passage #20.

2. Remove the medium and wash the cells twice with PBS. Lyse with HNTG lysis buffer. Collect all lysates and vortex mix them for 20-30 seconds.

3. Remove insoluble material by centrifugation (5-10 min at about 10,000×g).

4. Determine the protein concentration using BCA kit.

5. Partition lysate into 1 mg aliquots, store at −80° C.

Assay Procedure

1. Coat Corning 96-well ELISA plates with 2 μg/well purified L4 (or E 38) in 100 μl of PBS. Store overnight at 4° C.

2. Remove unbound proteins from wells by inverting the plate to remove the liquid. Wash one time with dH₂O, pat plate on paper towel to remove excess liquid.

3. Block plates with 150 μl blocking buffer per well. Incubate for 45-60 minutes with shaking at 4° C.

4. Remove the blocking buffer and wash the ELISA plate three times with dH₂O and one time with TBST. Pat plate on paper towel to remove excess liquid.

5. Dilute lysate in PBS to give final concentration of 50 μg/100 μl. Add 100 μl of diluted lysate to each well. Incubate with shaking at 4° C. overnight.

6. Remove unbound proteins from wells by inverting the plate. Wash as in step 4.

7. Add 80 μl of kinase buffer to wells (90 μl to negative control wells).

8. Dilute test compounds (normally 10-fold) into wells of a polypropylene plate containing 20% DMSO in TBS.

9. Add 10 μl of the diluted compounds to the ELISA wells containing immobilized flk-1 and shake. Control wells receive no compounds.

10. From stock 1 mM ATP, prepare 0.3 mM ATP solution in dH₂O (alternatively, kinase buffer may be used).

11. Add 10 μl of 0.3 mM ATP to all wells except the negative controls. Incubate for 60 min. at room temperature with shaking.

12. After 1 hr stop the kinase reaction by adding 11 μl 200 mM EDTA. Shake for 1-2 min.

13. Wash the ELISA plate 4 times with dH₂O and twice with TBST.

14. Add 100 μl of 1:5000 biotinylated 4G10:TBST to all wells. Incubate 45 min with shaking at room temperature.

15. While the above is incubating, add 50 μl of solutions A & B from the ABC kit to 10 ml of TBST. These solutions must be combined approximately 30 min prior to use.

16. Wash plates as in step 4.

17. Add 100 μl of the preformed A & B complex to all wells. Incubate 30 min with shaking at room temperature.

18. Wash plates as in step 4.

19. Add 100 μl turbo-TMB. Shake at room temperature for 10-15 min.

20. When the color in the positive control wells reaches an absorbance of about 0.35-0.4, stop the reaction with 100 μl of turbo-TMB stop solution.

21. Read plates on Dynatech MR7000 ELISA reader; test filter: 450 nM, reference filter: 410 nM.

HUV-EC-C Assay

The following protocol may also be used to measure a compound's activity against PDGF-R, FGF-R, VEGF, aFGF or Flk-1/KDR, all of which are naturally expressed by HUV-EC cells.

Day 0

1. Wash and trypsinize HUV-EC-C cells (human umbilical vein endothelial cells, (American Type Culture Collection; catalogue no. 1730 CRL). Wash with Dulbecco's phosphate-buffered saline (D-PBS; obtained from Gibco BRL; catalogue no. 14190-029) 2 times at about 1 ml/10 cm² of tissue culture flask. Trypsinize with 0.05% trypsin-EDTA in non-enzymatic cell dissociation solution (Sigma Chemical Company; catalogue no. C-1544). The 0.05% trypsin is made by diluting 0.25% trypsin/1 mM EDTA (Gibco; catalogue no. 25200-049) in the cell dissociation solution. Trypsinize with about 1 ml/25-30 cm² of tissue culture flask for about 5 minutes at 37° C. After cells have detached from the flask, add an equal volume of assay medium and transfer to a 50 ml sterile centrifuge tube (Fisher Scientific; catalogue no. 05-539-6).

2. Wash the cells with about 35 ml assay medium in the 50 ml sterile centrifuge tube by adding the assay medium, centrifuge for 10 minutes at approximately 200×g, aspirate the supernatant, and resuspend with 35 ml D-PBS. Repeat the wash two more times with D-PBS, resuspend the cells in about 1 ml assay medium/15 cm² of tissue culture flask. Assay medium consists of F12K medium (Gibco BRL; catalogue no. 21127-014) and 0.5% heat-inactivated fetal bovine serum. Count the cells with a Coulter Counter® (Coulter Electronics, Inc.) and add assay medium to the cells to obtain a concentration of 0.8-1.0×10⁵ cells/ml.

3. Add cells to 96-well flat-bottom plates at 100 μl/well or 0.8-1.0×10⁴ cells/well; incubate ˜24 h at 37° C., 5% CO₂.

Day 1

1. Make up two-fold test compound titrations in separate 96-well plates, generally 50 μM on down to 0 μM. Use the same assay medium as mentioned in day 0, step 2 above. Titrations are made by adding 90 μl/well of test compound at 200 μM (4×the final well concentration) to the top well of a particular plate column. Since the stock test compound is usually 20 mM in DMSO, the 200 1M drug concentration contains 2% DMSO.

A diluent made up to 2% DMSO in assay medium (F12K+0.5% fetal bovine serum) is used as diluent for the test compound titrations in order to dilute the test compound but keep the DMSO concentration constant. Add this diluent to the remaining wells in the column at 60 μl/well. Take 60 μl from the 120 μl of 200 μM test compound dilution in the top well of the column and mix with the 60 μl in the second well of the column. Take 60 μl from this well and mix with the 60 μl in the third well of the column, and so on until two-fold titrations are completed. When the next-to-the-last well is mixed, take 60 μl of the 120 μl in this well and discard it. Leave the last well with 60 μl of DMSO/media diluent as a non-test compound-containing control. Make 9 columns of titrated test compound, enough for triplicate wells each for: (1) VEGF (obtained from Pepro Tech Inc., catalogue no. 100-200; (2) endothelial cell growth factor (ECGF) (also known as acidic fibroblast growth factor, or aFGF) (obtained from Boehringer Mannheim Biochemica, catalogue no. 1439 600); or, (3) human PDGF B/B (1276-956, Boehringer Mannheim, Germany) and assay media control. ECGF comes as a preparation with sodium heparin.

2. Transfer 50 μl/well of the test compound dilutions to the 96-well assay plates containing the 0.8−10×10⁴ cells/100 μl/well of the HUV-EC-C cells from day 0 and incubate ˜2 h at 37° C., 5% CO₂.

3. In triplicate, add 50 μl/well of 80 μg/ml VEGF, 20 ng/ml ECGF, or media control to each test compound condition. As with the test compounds, the growth factor concentrations are 4×the desired final concentration. Use the assay media from day 0 step 2 to make the concentrations of growth factors. Incubate approximately 24 hours at 37° C., 5% CO₂. Each well will have 50 μl test compound dilution, 50 μl growth factor or media, and 100 μl cells, which calculates to 200 μl/well total. Thus the 4×concentrations of test compound and growth factors become 1×once everything has been added to the wells.

Day 2

1. Add ³H-thymidine (Amersham; catalogue no. TRK-686) at 1 μCi/well (10 μl/well of 100 μCi/ml solution made up in RPMI media+10% heat-inactivated fetal bovine serum) and incubate ˜24 h at 37° C., 5% CO₂. RPMI is obtained from Gibco BRL, catalogue no. 11875-051.

Day 3

1. Freeze plates overnight at −20° C.

Day 4

Thaw plates and harvest with a 96-well plate harvester (Tomtec Harvester 96®) onto filter mats (Wallac; catalogue no. 1205-401); read counts on a Wallac Betaplate™ liquid scintillation counter.

In vivo Animal Models

Xenograft Animal Models

The ability of human tumors to grow as xenografts in athymic mice (e.g., Balb/c, nu/nu) provides a useful in vivo model for studying the biological response to therapies for human tumors. Since the first successful xenotransplantation of human tumors into athymic mice, (Rygaard and Povlsen, 1969, Acta Pathol. Microbial. Scand. 77:758-760), many different human tumor cell lines (e.g., mammary, lung, genitourinary, gastrointestinal, head and neck, glioblastoma, bone, and malignant melanomas) have been transplanted and successfully grown in nude mice. The following assays may be used to determine the level of activity, specificity and effect of the different compounds of the present invention. Three general types of assays are useful for evaluating compounds: cellular/catalytic, cellular/biological and in vivo. The object of the cellular/catalytic assays is to determine the effect of a compound on the ability of a TK to phosphorylate tyrosines on a known substrate in a cell. The object of the cellular/biological assays is to determine the effect of a compound on the biological response stimulated by a TK in a cell. The object of the in vivo assays is to determine the effect of a compound in an animal model of a particular disorder such as cancer.

Suitable cell lines for subcutaneous xenograft experiments include C6 cells (glioma, ATCC #CCL 107), A375 cells (melanoma, ATCC #CRL 1619), A431 cells (epidermoid carcinoma, ATCC #CRL 1555), Calu 6 cells (lung, ATCC #HTB 56), PC3 cells (prostate, ATCC #CRL 1435), SKOV3TP5 cells and NIH 3T3 fibroblasts genetically engineered to overexpress EGFR, PDGFR, IGF-1R or any other test kinase. The following protocol can be used to perform xenograft experiments:

Female athymic mice (BALB/c, nu/nu) are obtained from Simonsen Laboratories (Gilroy, Calif.). All animals are maintained under clean-room conditions in Micro-isolator cages with Alpha-dri bedding. They receive sterile rodent chow and water ad libitum.

Cell lines are grown in appropriate medium (for example, MEM, DMEM, Ham's F10, or Ham's F12 plus 5%-10% fetal bovine serum (FBS) and 2 mM glutamine (GLN)). All cell culture media, glutamine, and fetal bovine serum are purchased from Gibco Life Technologies (Grand Island, N.Y.) unless otherwise specified. All cells are grown in a humid atmosphere of 90-95% air and 5-10% CO₂ at 37° C. All cell lines are routinely subcultured twice a week and are negative for mycoplasma as determined by the Mycotect method (Gibco).

Cells are harvested at or near confluency with 0.05% Trypsin-EDTA and pelleted at 450×g for 10 min. Pellets are resuspended in sterile PBS or media (without FBS) to a particular concentration and the cells are implanted into the hindflank of the mice (8-10 mice per group, 2−10×10⁶ cells/animal). Tumor growth is measured over 3 to 6 weeks using venier calipers. Tumor volumes are calculated as a product of length×width×height unless otherwise indicated. P values are calculated using the Students t-test. Test compounds in 50-100 μL excipient (DMSO, or VPD:D5W) can be delivered by IP injection at different concentrations generally starting at day one after implantation.

Tumor Invasion Model

The following tumor invasion model has been developed and may be used for the evaluation of therapeutic value and efficacy of the compounds identified to selectively inhibit KDR/FLK-1 receptor.

Procedure

8 week old nude mice (female) (Simonsen Inc.) are used as experimental animals. Implantation of tumor cells can be performed in a laminar flow hood. For anesthesia, Xylazine/Ketamine Cocktail (100 mg/kg ketamine and 5 mg/kg Xylazine) are administered intraperitoneally. A midline incision is done to expose the abdominal cavity (approximately 1.5 cm in length) to inject 10⁷ tumor cells in a volume of 100 μl medium. The cells are injected either into the duodenal lobe of the pancreas or under the serosa of the colon. The peritoneum and muscles are closed with a 6-0 silk continuous suture and the skin is closed by using wound clips. Animals are observed daily.

Analysis

After 2-6 weeks, depending on gross observations of the animals, the mice are sacrificed, and the local tumor metastases to various organs (lung, liver, brain, stomach, spleen, heart, muscle) are excised and analyzed (measurement of tumor size, grade of invasion, immunochemistry, in situ hybridization determination, etc.).

Measurement Of Cell Toxicity

Therapeutic compounds should be more potent in inhibiting receptor tyrosine kinase activity than in exerting a cytotoxic effect. A measure of the effectiveness and cell toxicity of a compound can be obtained by determining the therapeutic index; i.e., IC₅₀/LD₅₀. IC₅₀, the dose required to achieve 50% inhibition, can be measured using standard techniques such as those described herein. LD₅₀, the dosage which results in 50% toxicity, can also be measured by standard techniques as well(Mossman, 1983, J. Immunol. Methods, 65:55-63), by measuring the amount of LDH released (Korzeniewski and Callewaert, 1983, J. Immunol. Methods, 64:313; Decker and Lohmann-Matthes, 1988, J. Immunol. Methods, 115:61), or by measuring the lethal dose in animal models. Compounds with a large therapeutic index are preferred. The therapeutic index should be greater than 2, preferably at least 10, more preferably at least 50.

### Conclusion

Thus, it will be appreciated that 3-heteroarylidenyl-2-indolinones are expected to have a beneficial effect on the chemotherapeutic efficacy of various chemotherapeutic agents, in particular fluorinated pyrimidine compounds. Furthermore 3-[(2,4-Dimethylpyrrol-5-yl)methylene]-2-indolinone together with fluorouracil or fluorouracil/leucovorin is expected to be an effective chemotherapeutic combination for the treatment of colorectal cancer.

It will also be appreciated that the compounds, methods and pharmacological compositions of the present invention are expected to modulate RTK and CTK activity and therefore to be effective as therapeutic agents against RTK- and CTK-related disorders.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments and are exemplary and are not intended as limitations on the scope of the invention. Changes therein and other uses will occur to those skilled in the art which are encompassed within the spirit of the invention and are defined by the scope of the claims.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

Other embodiments are presented within the following claims.

What is claimed is:

1. A method for treating cancer comprising administering to a patient in need of such treatment a therapeutically effective amount of a fluorinated pyrimidine chemotherapeutic agent and a therapeutically effective amount of a compound having the chemical structure: 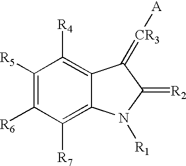
wherein:
R₁ is H or alkyl;
R₂ is O or S;
R₃ is hydrogen;
R₄, R₅, R₆, and R₇ are each independently selected from the group consisting of hydrogen, alkyl, alkoxy, aryl, aryloxy, alkaryl, alkaryloxy, halogen, trihalomethyl, S(O)R, SO₂NRR′, SO₃R, SR, NO₂, NRR′, OH, CN, C(O)R, OC(O)R, (CH₂)ₙCO₂R, and CONRR′;
A is a five membered heteroaryl ring selected from the group consisting of thiophene, pyrrole, pyrazole, imidazole, 1,2,3-triazole, 1,2,4-triazole, oxazole, isoxazole, thiazole, isothiazole, 2-sulfonylfuran, 4-alkylfuran, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole, and tetrazole, optionally substituted at one or more positions with alkyl, alkoxy, aryl, aryloxy, alkaryl, alkaryloxy, halogen, trihalomethyl, S(O)R, SO₂NRR′, SO₃R, SR, NO₂, NRR′, OH, CN, C(O)R, OC(O)R, (CH₂)ₙCO₂R, or CONRR′;
n is 0-3; and,
R and R′ are independently selected from the group consisting of H, alkyl or aryl; or,
a physiologically acceptable salt or prodrug thereof.

2. The method of claim 1 wherein said compound is selected from the group consisting of 5-hydroxy-3-[(2,4-dimethylpyrrol-5-yl)methylidenyl]-2-indolinone, 4-methyl-5-(2-oxo-1,2-dihydroindol-3-ylidenemethyl)-1H-pyrrole-2-carboxylic acid, 4-methyl-5-(2-oxo-1,2-dihydroindol-3-ylidenemethyl)-1H-pyrrole-2-carboxylic acid methyl ester, 3-(5-hydroxymethyl-3-methyl-1H-pyrrol-2-ylmethylene)-1,3-dihydroindole-2-one and 4-methyl-5-(2-oxo-1,2-dihydroindol-3-ylidenemethyl)-1H-pyrrole-2-carbaldehyde or a physiologically acceptable salt or prodrug thereof.

3. The method of claim 1 wherein said compound is 3-[4-(2-carboxyethyl-3,5-dimethylpyrrol-2-yl)methylidenyl]-2-indolinone.

4. The method of claim 1 wherein said compound is 3-[(2,4-dimethylpyrrol-5-yl)methylidenyl]-2-indolinone.

5. The method of claim 1 wherein said fluorinated pyrimidine chemotherapeutic agent is selected from the group consisting of carmofur, doxifluridine, fluorouracil, floxuridine, tegafur, capecitabine and uracil-ftorafur.

6. The method of claim 1 wherein said fluorinated pyrimidine chemotherapeutic agent is fluorouracil.

7. The method of claim 6 further comprising administering a therapeutically effective amount of leucovorin to said patient.

8. The method of claim 1 wherein said cancer is selected from the group consisting of breast cancer, gastric cancer, ovarian cancer, renal cancer, hepatic cancer, pancreatic cancer, bladder cancer, prostate cancer and colorectal cancer.

9. A method for treating colorectal cancer comprising administering to a patient in need of such treatment a therapeutically effective amount of fluorouracil and a therapeutically effective amount of 3-[(2,4-dimethylpyrrol-5-yl)methylidenyl]-2-indolinone.

10. The method of claim 9 further comprising administering a therapeutically effective amount of leucovorin to said patient.

11. The method of claim 9 wherein said therapeutically effective amount of said fluoruracil comprises from about 400 mg/m² to about 500 mg/m².

12. The method of claim 9 wherein said therapeutically effective amount of said fluorouracil is administered parenterally.

13. The method of claim 9 wherein said therapeutically effective amount of said 3-[(2,4-dimethylpyrrol-5-yl)methylidenyl]-2-indolinone comprises from about 4 mg/m² to about 190 mg/m² per treatment.

14. The method of claim 9 wherein said therapeutically effective amount of said 3-[(2,4-dimethylpyrrol-5-yl)methylidenyl]-2-indolinone comprises from about 72 mg/m² to about 145 mg/m² per treatment.

15. A method for treating cancer comprising a combination of 3-[(2,4-dimethylpyrrol-5-yl)methylidenyl]-2-indolinone and at least one other chemotherapeutic agent.

16. The method of claim 15 wherein said other chemotherapeutic agent is selected from the group consisting of capecitabine, 5-FU, UFT, carboplatin, cisplatin, oxaliplatin, paclitaxel, docetaxel, a polyglutamated taxane, irinotecan, thalidomide, a COX-2 inhibitor, tamoxifen, leuprolide, angiostatin, endostatin, a matrix metalloprotease inhibitor, an interferon, doxorubicin, liposomal doxorubicin, daunorubicin, metoxantrone, estramucine and a vinca alkaloid, or combinations thereof.

17. A 3-heteroarylidenyl-2-indolinone compound selected from the group consisting of 5-hydroxy-3-[(2,4-dimethylpyrrol-5-yl)methylidenyl]-2-indolinone, 4-methyl-5-(2-oxo-1,2-dihydroindol-3-ylidenemethyl)-1H-pyrrole-2-carboxylic acid, 4-methyl-5-(2-oxo-1,2-dihydroindol-3-ylidenemethyl)-1H-pyrrole-2-carboxylic acid methyl ester, 3-(5-hydroxymethyl-3-methyl-1H-pyrrol-2-ylmethylene)-1,3-dihydroindole-2-one and 4-methyl-5-(2-oxo-1,2-dihydroindol-3-ylidenemethyl)-1H-pyrrole-2-carbaldehyde or a physiologically acceptable salt or prodrug thereof.

18. A method for the modulation of the catalytic activity of a protein kinase comprising contacting said protein kinase with a compound, salt or prodrug of claim 17.

19. The method of claim 18 wherein said protein kinase is selected from the group consisting of receptor protein tyrosine kinase, cellular tyrosine kinase and serine-threonine kinase.

20. A pharmaceutical composition comprising:
a compound, salt or prodrug of claim 17; and,
a pharmaceutically acceptable carrier or excipient.

21. A method for treating or preventing a protein kinase related disorder in an patient comprising administering a therapeutically effective amount of a compound, salt or prodrug of claim 17 to said patient.

22. The method of claim 21 wherein said protein kinase related disorder is selected from the group consisting of a receptor protein tyrosine kinase related disorder, a cellular tyrosine kinase disorder and a serine-threonine kinase related disorder.

23. The method of claim 21 wherein said protein kinase related disorder is selected from the group consisting of an EGFR related disorder, a PDGFR related disorder, an IGFR related disorder and a flk related disorder.

24. The method of claim 21 wherein said protein kinase related disorder is a cancer selected from the group consisting of squamous cell carcinoma, astrocytoma, glioblastoma, lung cancer, bladder cancer, head and neck cancer, melanoma, ovarian cancer, prostate cancer, breast cancer, small-cell lung cancer and glioma.

25. The method of claim 21 wherein said protein kinase related disorder is selected from the group consisting of diabetes, an autoimmune disorder, a hyperproliferation disorder, restenosis, fibrosis, psoriasis, osteoarthritis, rheumatoid arthritis, an inflammatory disorder and angiogenesis.

26. A method for treating cancer comprising administering to a patient in need thereof a therapeutically effective amount of gemcitabine and a therapeutically effective amount of 3-[(2,4-dimethylpyrrol-5-yl)methylidenyl]-2-indolinone.

27. The method of claim 26 wherein said cancer is pancreatic cancer.

28. The method of claim 26 further comprising a therapeutically effective amount of paclitaxel, carboplatin, liposomal doxorubicin, or topotecan.

29. The method of claim 28 wherein said cancer is selected from the group consisting of ovarian, small-cell lung and kidney cancer.
